SEQUENCE LISTING (191 sequences)

SEQ ID NO: 1 - protein, 33 aa, synthetic construct
  VARIANT 2: note = MISC_FEATURE - Xaa is Aib or G
  VARIANT 5: note = MISC_FEATURE - Xaa is T or S, wherein at least one of Xaa at positions 5 or 7 isT
  VARIANT 7: note = MISC_FEATURE - Xaa is T or S, wherein at least one of Xaa at positions 5 or 7 isT
  VARIANT 8: note = MISC_FEATURE - Xaa is S, E or D
  VARIANT 10: note = MISC_FEATURE - Xaa is L, M, V or a residue of K, k, R, Orn, Dap or Dab in whichthe side chain is conjugated to a substituent
  VARIANT 11: note = MISC_FEATURE - Xaa is A, N or S
  VARIANT 15: note = MISC_FEATURE - Xaa is D or E
  VARIANT 16: note = MISC_FEATURE - Xaa is G, E, A or a residue of K, k, R, Orn, Dap or Dab in whichthe side chain is conjugated to a substituent
  VARIANT 17: note = MISC_FEATURE - Xaa is Q, E, K, L or a residue of K, k, R, Orn, Dap or Dab inwhich the side chain is conjugated to a substituent
  VARIANT 19: note = MISC_FEATURE - Xaa is A, V or S
  VARIANT 20: note = MISC_FEATURE - Xaa is R, K or a residue of K, k, R, Orn, Dap or Dab in which theside chain is conjugated to a substituent
  VARIANT 21: note = MISC_FEATURE - Xaa is D, L or E
  VARIANT 24: note = MISC_FEATURE - Xaa is A, N or S
  VARIANT 27: note = MISC_FEATURE - Xaa is I, Q, K, H or Y
  VARIANT 28: note = MISC_FEATURE - Xaa is Q, E, A, H, Y, L, K, R or S
  VARIANT 29: note = MISC_FEATURE - Xaa is H, Y, K or Q
  VARIANT 33: note = MISC_FEATURE - Xaa is D or E
  REGION 1..33: note = GLP-1/GLP-2 dual agonist
HXEGXFXXEXXTILXXXAXXXFIXWLXXXKITX

SEQ ID NO: 2 - protein, 33 aa, synthetic construct
  REGION 1..33: note = GLP-1/GLP-2 dual agonist peptide
  VARIANT 2: note = MISC_FEATURE - Xaa is Aib or G
  VARIANT 5: note = MISC_FEATURE - Xaa is T or S, wherein at least one of Xaa at positions 5 or 7 isT
  VARIANT 7: note = MISC_FEATURE - Xaa is T or S, wherein at least one of Xaa at positions 5 or 7 isT
  VARIANT 16: note = MISC_FEATURE - Xaa is G or a residue of K, k, R, Orn, Dap or Dab in which theside chain is conjugated to a substituent
  VARIANT 17: note = MISC_FEATURE - Xaa is Q, E, K, L or a residue of K, k, R, Orn, Dap or Dab inwhich the side chain is conjugated to a substituent
  VARIANT 21: note = MISC_FEATURE - Xaa is D or L
  VARIANT 28: note = MISC_FEATURE - Xaa is Q, E, A, H, Y, L, K, R or S
  VARIANT 29: note = MISC_FEATURE - Xaa is H, Y or Q
HXEGXFXSELATILDXXAARXFIAWLIXXKITD

SEQ ID NO: 3 - protein, 33 aa, synthetic construct
  REGION 1..33: note = GLP-1/GLP-2 dual agonist peptide
  SITE 2: note = MISC_FEATURE - Xaa is Aib
  VARIANT 5: note = MISC_FEATURE - Xaa is T or S, wherein at least one of Xaa at positions 5 or 7 isT
  VARIANT 7: note = MISC_FEATURE - Xaa is T or S, wherein at least one of Xaa at positions 5 or 7 isT
  VARIANT 10: note = MISC_FEATURE - Xaa is L or a residue of K, k, R, Orn, Dap or Dab in which theside chain is conjugated to a substituent
  VARIANT 16: note = MISC_FEATURE - Xaa is G, E, A or a residue of K, k, R, Orn, Dap or Dab in whichthe side chain is conjugated to a substituent
  VARIANT 17: note = MISC_FEATURE - Xaa is Q, E, K, L or a residue of K, k, R, Orn, Dap or Dab inwhich the side chain is conjugated to a substituent
  VARIANT 20: note = MISC_FEATURE - Xaa is R or a residue of K, k, R, Orn, Dap or Dab in which theside chain is conjugated to a substituent
  VARIANT 21: note = MISC_FEATURE - Xaa is D or L
  VARIANT 28: note = MISC_FEATURE - Xaa is E, A or Q
  VARIANT 29: note = MISC_FEATURE - Xaa is H, Y or Q
HXEGXFXSEXATILDXXAAXXFIAWLIXXKITD

SEQ ID NO: 4 - protein, 33 aa, synthetic construct; identical to SEQ ID NO: 2
  REGION 1..33: note = GLP-1/GLP-2 dual agonist
  SITE 2: note = MISC_FEATURE - Xaa is Aib
  SITE 5: note = MISC_FEATURE - Xaa is T, wherein at least one of Xaa at positions 5 and 7 is T
  SITE 7: note = MISC_FEATURE - Xaa is T, wherein at least one of Xaa at positions 5 and 7 is T
  VARIANT 16: note = MISC_FEATURE - Xaa is G or a residue of K, k, R, Orn, Dap or Dab in which theside chain is conjugated to a substituent
  VARIANT 17: note = MISC_FEATURE - Xaa is E, K, L or a residue of K, k, R, Orn, Dap or Dab in whichthe side chain is conjugated to a substituent
  VARIANT 21: note = MISC_FEATURE - Xaa is D or L
  VARIANT 28: note = MISC_FEATURE - Xaa is E or A
  VARIANT 29: note = MISC_FEATURE - Xaa is H, Y or Q

SEQ ID NO: 5 - protein, 33 aa, synthetic construct
  REGION 1..33: note = GLP-1/GLP-2 dual agonist
  SITE 2: note = MISC_FEATURE - Xaa is Aib
  VARIANT 5: note = MISC_FEATURE - Xaa is T or S, wherein at least one of Xaa at positions 5 and 7is T
  VARIANT 7: note = MISC_FEATURE - Xaa is T or S, wherein at least one of Xaa at positions 5 and 7is T
  VARIANT 16: note = MISC_FEATURE - Xaa is a residue of K, k, R, Orn, Dap or Dab in which the sidechain is conjugated to a substituent
  VARIANT 28: note = MISC_FEATURE - Xaa is Q, E, A, H, Y, L, K, R or S
  VARIANT 29: note = MISC_FEATURE - Xaa is H, Y or Q
HXEGXFXSELATILDXQAARDFIAWLIXXKITD

SEQ ID NO: 6 - skipped

SEQ ID NO: 7 - protein, 6 aa, synthetic construct
  REGION 1..6: note = Linker sequence
EKEKEK

SEQ ID NO: 8 - protein, 6 aa, synthetic construct
  REGION 1..6: note = Linker sequence
  SITE 2: note = MISC_FEATURE - Lys is D-Lys
  SITE 4: note = MISC_FEATURE - Lys is D-Lys
  SITE 6: note = MISC_FEATURE - Lys is D-Lys
EKEKEK

SEQ ID NO: 9 - protein, 6 aa, synthetic construct
  REGION 1..6: note = Linker sequence
AKAAEK

SEQ ID NO: 10 - protein, 6 aa, synthetic construct
  REGION 1..6: note = Linker sequence
AKEKEK

SEQ ID NO: 11 - protein, 6 aa, synthetic construct
  REGION 1..6: note = Linker sequence
ATILEK

SEQ ID NO: 12 - protein, 6 aa, synthetic construct
  REGION 1..6: note = Linker sequence
  VARIANT 6: note = MISC_FEATURE - Xaa is a residue of K, k, R, Orn, Dap or Dab in which the sidechain is conjugated to a substituent
EKEKEX

SEQ ID NO: 13 - protein, 6 aa, synthetic construct
  REGION 1..6: note = Linker sequence
  SITE 2: note = MISC_FEATURE - Lys is D-Lys
  SITE 4: note = MISC_FEATURE - Lys is D-Lys
  VARIANT 6: note = a residue of K, k, R, Orn, Dap or Dab in which the side chain is conjugated to a substituent
EKEKEX

SEQ ID NO: 14 - protein, 6 aa, synthetic construct
  REGION 1..6: note = Linker sequence
  VARIANT 6: note = MISC_FEATURE - Xaa is a residue of K, k, R, Orn, Dap or Dab in which the sidechain is conjugated to a substituent
AKAAEX

SEQ ID NO: 15 - protein, 6 aa, synthetic construct
  REGION 1..6: note = Linker sequence
  VARIANT 6: note = MISC_FEATURE - Xaa is a residue of K, k, R, Orn, Dap or Dab in which the sidechain is conjugated to a substituent
AKEKEX

SEQ ID NO: 16 - protein, 6 aa, synthetic construct
  REGION 1..6: note = Linker sequence
  VARIANT 6: note = MISC_FEATURE - Xaa is a residue of K, k, R, Orn, Dap or Dab in which the sidechain is conjugated to a substituent
ATILEX

SEQ ID NO: 17 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFSSELATILDXEAARDFIAWLIEHKITD

SEQ ID NO: 18 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXEAARDFIAWLIEHKITD

SEQ ID NO: 19 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFTSELATILDXEAARDFIAWLIEHKITD

SEQ ID NO: 20 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFSSELATILDXKAARDFIAWLIEHKITD

SEQ ID NO: 21 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXKAARDFIAWLIEHKITD

SEQ ID NO: 22 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFTSELATILDXKAARDFIAWLIEHKITD

SEQ ID NO: 23 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Xaa is a residue of K, k, R, Orn, Dap or Dab in which the sidechain is conjugated to a substituent
HXEGTFSSELATILDGXAARDFIAWLIEHKITD

SEQ ID NO: 24 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Xaa is a residue of K, k, R, Orn, Dap or Dab in which the sidechain is conjugated to a substituent
HXEGSFTSELATILDGXAARDFIAWLIEHKITD

SEQ ID NO: 25 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Xaa is a residue of K, k, R, Orn, Dap or Dab in which the sidechain is conjugated to a substituent
HXEGTFTSELATILDGXAARDFIAWLIEHKITD

SEQ ID NO: 26 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFSSELATILDXLAARDFIAWLIEHKITD

SEQ ID NO: 27 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXLAARDFIAWLIEHKITD

SEQ ID NO: 28 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFTSELATILDXLAARDFIAWLIEHKITD

SEQ ID NO: 29 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFSSELATILDXLAARDFIAWLIAHKITD

SEQ ID NO: 30 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXLAARDFIAWLIAHKITD

SEQ ID NO: 31 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFTSELATILDXLAARDFIAWLIAHKITD

SEQ ID NO: 32 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFTSELATILDXEAARLFIAWLIEHKITD

SEQ ID NO: 33 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFSSELATILDXQAARDFIAWLIQHKITD

SEQ ID NO: 34 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIQHKITD

SEQ ID NO: 35 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFTSELATILDXQAARDFIAWLIQHKITD

SEQ ID NO: 36 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFSSELATILDXQAARDFIAWLIEHKITD

SEQ ID NO: 37 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFSSELATILDXQAARDFIAWLIAHKITD

SEQ ID NO: 38 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIAHKITD

SEQ ID NO: 39 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFTSELATILDXQAARDFIAWLIAHKITD

SEQ ID NO: 40 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIEHKITD

SEQ ID NO: 41 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFTSELATILDXQAARDFIAWLIEHKITD

SEQ ID NO: 42 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIHHKITD

SEQ ID NO: 43 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIYHKITD

SEQ ID NO: 44 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLILHKITD

SEQ ID NO: 45 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIKHKITD

SEQ ID NO: 46 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIRHKITD

SEQ ID NO: 47 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLISHKITD

SEQ ID NO: 48 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLQQHKITD

SEQ ID NO: 49 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLYQHKITD

SEQ ID NO: 50 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLKQHKITD

SEQ ID NO: 51 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIQQKITD

SEQ ID NO: 52 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGSFTSELATILDXQAARDFIAWLIQYKITD

SEQ ID NO: 53 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Xaa is a residue of K, k, R, Orn, Dap or Dab in which the sidechain is conjugated to a substituent
HXEGTFSSELSTILEXQASREFIAWLIAYKITE

SEQ ID NO: 54 - protein, 39 aa, synthetic construct
  REGION 1..39: note = GLP-1/GLP-2 dual agonist peptide
  SITE 2: note = MISC_FEATURE - Xaa is Aib
  SITE 34..38: note = Lys is D-Lys
  VARIANT 39: note = MISC_FEATURE - Xaa is a residue of K, k, R, Orn, Dap or Dab in which the sidechain is conjugated to a substituent
HXEGTFSSELATILDEQAARDFIAWLIAHKITDKKKKKX

SEQ ID NO: 55 - protein, 39 aa, synthetic construct
  REGION 1..39: note = GLP-1/GLP-2 dual agonist peptide
  SITE 2: note = MISC_FEATURE - Xaa is Aib
  SITE 34..38: note = Lys is D-Lys
  VARIANT 39: note = MISC_FEATURE - Xaa is a residue of K, k, R, Orn, Dap or Dab in which the sidechain is conjugated to a substituent
HXEGTFTSELATILDEQAARDFIAWLIAHKITDKKKKKX

SEQ ID NO: 56 - protein, 39 aa, synthetic construct
  REGION 1..39: note = GLP-1/GLP-2 dual agonist peptide
  SITE 2: note = MISC_FEATURE - Xaa is Aib
  SITE 34..38: note = Lys is D-Lys
  VARIANT 39: note = MISC_FEATURE - Xaa is a residue of K, k, R, Orn, Dap or Dab in which the sidechain is conjugated to a substituent
HXEGSFTSELATILDEQAARDFIAWLIEHKITDKKKKKX

SEQ ID NO: 57 - protein, 33 aa, synthetic construct
  REGION 1..33: note = GLP-1/GLP-2 dual agonist peptide
  SITE 2: note = MISC_FEATURE - Xaa is Aib
  VARIANT 10: note = MISC_FEATURE - Xaa is a residue of K, k, R, Orn, Dap or Dab in which the sidechain is conjugated to a substituent
HXEGSFTSEXATILDEQAARDFIAWLIEHKITD

SEQ ID NO: 58 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Xaa is a residue of K, k, R, Orn, Dap or Dab in which the sidechain is conjugated to a substituent
HXEGSFTSELATILEGXAARDFIAWLIEHKITD

SEQ ID NO: 59 - protein, 33 aa, synthetic construct
  REGION 1..33: note = GLP-1/GLP-2 dual agonist peptide
  SITE 2: note = MISC_FEATURE - Xaa is Aib
  VARIANT 20: note = MISC_FEATURE - Xaa is a residue of K, k, R, Orn, Dap or Dab in which the sidechain is conjugated to a substituent
HXEGSFTSELATILDEQAAXDFIAWLIEHKITD

SEQ ID NO: 60 - protein, 33 aa, synthetic construct
  REGION 1..33: note = GLP-1/GLP-2 dual agonist peptide
  SITE 2: note = MISC_FEATURE - Xaa is Aib
  VARIANT 20: note = MISC_FEATURE - Xaa is a residue of K, k, R, Orn, Dap or Dab in which the sidechain is conjugated to a substituent
HXEGTFTSELATILDEQAAXDFIAWLIEHKITD

SEQ ID NO: 61 - protein, 33 aa, synthetic construct
  REGION 1..33: note = GLP-1/GLP-2 dual agonist peptide
  SITE 2: note = MISC_FEATURE - Xaa is Aib
  VARIANT 10: note = MISC_FEATURE - Xaa is a residue of K, k, R, Orn, Dap or Dab in which the sidechain is conjugated to a substituent
HXEGTFTSEXATILDEQAARDFIAWLIEHKITD

SEQ ID NO: 62 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Xaa is a residue of K, k, R, Orn, Dap or Dab in which the sidechain is conjugated to a substituent
HXEGSFTSELATILDAXAARDFIAWLIEHKITD

SEQ ID NO: 63 - protein, 33 aa, synthetic construct
  REGION 1..33: note = GLP-1/GLP-2 dual agonist peptide
  SITE 2: note = MISC_FEATURE - Xaa is Aib
  VARIANT 20: note = MISC_FEATURE - Xaa is a residue of K, k, R, Orn, Dap or Dab in which the sidechain is conjugated to a substituent
HXEGSFTSELATILDAKAAXDFIAWLIEHKITD

SEQ ID NO: 64 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFSSELATILDKEAARDFIAWLIEHKITD

SEQ ID NO: 65 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKEAARDFIAWLIEHKITD

SEQ ID NO: 66 - protein, 33 aa, synthetic construct; identical to SEQ ID NO: 19
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-

SEQ ID NO: 67 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFSSELATILDKKAARDFIAWLIEHKITD

SEQ ID NO: 68 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKKAARDFIAWLIEHKITD

SEQ ID NO: 69 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFTSELATILDKKAARDFIAWLIEHKITD

SEQ ID NO: 70 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Lys is L-Lys in which the side chain is conjugated to thesubstituent Z1- or Z1Z2-
HXEGTFSSELATILDGKAARDFIAWLIEHKITD

SEQ ID NO: 71 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Lys is L-Lys in which the side chain is conjugated to thesubstituent Z1- or Z1Z2-
HXEGSFTSELATILDGKAARDFIAWLIEHKITD

SEQ ID NO: 72 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Lys is L-Lys in which the side chain is conjugated to thesubstituent Z1- or Z1Z2-
HXEGTFTSELATILDGKAARDFIAWLIEHKITD

SEQ ID NO: 73 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFSSELATILDKLAARDFIAWLIEHKITD

SEQ ID NO: 74 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKLAARDFIAWLIEHKITD

SEQ ID NO: 75 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFTSELATILDKLAARDFIAWLIEHKITD

SEQ ID NO: 76 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFSSELATILDKLAARDFIAWLIAHKITD

SEQ ID NO: 77 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKLAARDFIAWLIAHKITD

SEQ ID NO: 78 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFTSELATILDKLAARDFIAWLIAHKITD

SEQ ID NO: 79 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFTSELATILDKEAARLFIAWLIEHKITD

SEQ ID NO: 80 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFSSELATILDKQAARDFIAWLIQHKITD

SEQ ID NO: 81 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLIQHKITD

SEQ ID NO: 82 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFTSELATILDKQAARDFIAWLIQHKITD

SEQ ID NO: 83 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFSSELATILDKQAARDFIAWLIEHKITD

SEQ ID NO: 84 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFSSELATILDKQAARDFIAWLIAHKITD

SEQ ID NO: 85 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLIAHKITD

SEQ ID NO: 86 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFTSELATILDKQAARDFIAWLIAHKITD

SEQ ID NO: 87 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLIEHKITD

SEQ ID NO: 88 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFTSELATILDKQAARDFIAWLIEHKITD

SEQ ID NO: 89 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLIHHKITD

SEQ ID NO: 90 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLIYHKITD

SEQ ID NO: 91 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLILHKITD

SEQ ID NO: 92 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2
HXEGSFTSELATILDKQAARDFIAWLIKHKITD

SEQ ID NO: 93 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLIRHKITD

SEQ ID NO: 94 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLISHKITD

SEQ ID NO: 95 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLQQHKITD

SEQ ID NO: 96 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLYQHKITD

SEQ ID NO: 97 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLKQHKITD

SEQ ID NO: 98 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLIQQKITD

SEQ ID NO: 99 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGSFTSELATILDKQAARDFIAWLIQYKITD

SEQ ID NO: 100 - protein, 33 aa, synthetic construct
  REGION 1..33: note = GLP-1/GLP-2 dual agonist peptide
  SITE 2: note = MISC_FEATURE - Xaa is Aib
  VARIANT 16: note = MISC_FEATURE - Lys is L-Lys in which the side chain is conjugated to thesubstituent Z1- or Z1Z2-
HXEGTFSSELSTILEKQASREFIAWLIAYKITE

SEQ ID NO: 101 - protein, 39 aa, synthetic construct
  REGION 1..39: note = GLP-1/GLP-2 dual agonist peptide
  SITE 2: note = MISC_FEATURE - Xaa is Aib
  SITE 34..38: note = Lys is D-Lys
  VARIANT 39: note = MISC_FEATURE - Lys is D-Lys in which the side chain is conjugated to thesubstituent Z1- or Z1Z2-
HXEGTFSSELATILDEQAARDFIAWLIAHKITDKKKKKK

SEQ ID NO: 102 - protein, 39 aa, synthetic construct
  REGION 1..39: note = GLP-1/GLP-2 dual agonist peptide
  SITE 2: note = MISC_FEATURE - Xaa is Aib
  SITE 34..38: note = Lys is D-Lys
  VARIANT 39: note = MISC_FEATURE - Lys is D-Lys in which the side chain is conjugated to thesubstituent Z1- or Z1Z2-
HXEGTFTSELATILDEQAARDFIAWLIAHKITDKKKKKK

SEQ ID NO: 103 - protein, 39 aa, synthetic construct
  REGION 1..39: note = GLP-1/GLP-2 dual agonist peptide
  SITE 2: note = MISC_FEATURE - Xaa is Aib
  SITE 34..38: note = Lys is D-Lys
  VARIANT 39: note = MISC_FEATURE - Lys is D-Lys in which the side chain is conjugated to thesubstituent Z1- or Z1Z2-
HXEGSFTSELATILDEQAARDFIAWLIEHKITDKKKKKK

SEQ ID NO: 104 - protein, 33 aa, synthetic construct
  REGION 1..33: note = GLP-1/GLP-2 dual agonist peptide
  SITE 2: note = MISC_FEATURE - Xaa is Aib
  VARIANT 10: note = MISC_FEATURE - Lys is L-Lys in which the side chain is conjugated to thesubstituent Z1- or Z1Z2-
HXEGSFTSEKATILDEQAARDFIAWLIEHKITD

SEQ ID NO: 105 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Lys is L-Lys in which the side chain is conjugated to thesubstituent Z1- or Z1Z2-
HXEGSFTSELATILEGKAARDFIAWLIEHKITD

SEQ ID NO: 106 - protein, 33 aa, synthetic construct
  REGION 1..33: note = GLP-1/GLP-2 dual agonist peptide
  SITE 2: note = MISC_FEATURE - Xaa is Aib
  VARIANT 20: note = MISC_FEATURE - Lys is L-Lys in which the side chain is conjugated to thesubstituent Z1- or Z1Z2-
HXEGSFTSELATILDEQAAKDFIAWLIEHKITD

SEQ ID NO: 107 - protein, 33 aa, synthetic construct
  REGION 1..33: note = GLP-1/GLP-2 dual agonist peptide
  SITE 2: note = MISC_FEATURE - Xaa is Aib
  VARIANT 20: note = MISC_FEATURE - Lys is L-Lys in which the side chain is conjugated to thesubstituent Z1- or Z1Z2-
HXEGTFTSELATILDEQAAKDFIAWLIEHKITD

SEQ ID NO: 108 - protein, 33 aa, synthetic construct
  REGION 1..33: note = GLP-1/GLP-2 dual agonist peptide
  SITE 2: note = MISC_FEATURE - Xaa is Aib
  VARIANT 10: note = MISC_FEATURE - Lys is L-Lys in which the side chain is conjugated to thesubstituent Z1- or Z1Z2-
HXEGTFTSEKATILDEQAARDFIAWLIEHKITD

SEQ ID NO: 109 - protein, 33 aa, synthetic construct
  REGION 1..33: note = GLP-1/GLP-2 dual agonist peptide
  SITE 2: note = MISC_FEATURE - Xaa is Aib
  VARIANT 17: note = MISC_FEATURE - Lys is L-Lys in which the side chain is conjugated to thesubstituent Z1- or Z1Z2-
HXEGSFTSELATILDAKAARDFIAWLIEHKITD

SEQ ID NO: 110 - protein, 33 aa, synthetic construct
  REGION 1..33: note = GLP-1/GLP-2 dual agonist peptide
  SITE 2: note = MISC_FEATURE - Xaa is Aib
  VARIANT 20: note = MISC_FEATURE - Lys is L-Lys in which the side chain is conjugated to thesubstituent Z1- or Z1Z2-
HXEGSFTSELATILDAKAAKDFIAWLIEHKITD

SEQ ID NO: 111 - protein, 33 aa, synthetic construct; identical to SEQ ID NO: 64
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 112 - protein, 33 aa, synthetic construct; identical to SEQ ID NO: 65
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 113 - protein, 33 aa, synthetic construct
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)
HXEGTFTSELATILDKEAARDFIAWLIEHKITD

SEQ ID NO: 114 - protein, 33 aa, synthetic construct; identical to SEQ ID NO: 67
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 115 - protein, 33 aa, synthetic construct; identical to SEQ ID NO: 68
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 116 - protein, 33 aa, synthetic construct; identical to SEQ ID NO: 69
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 117 - protein, 33 aa, synthetic construct; identical to SEQ ID NO: 70
  REGION 1..33: note = GLP-1/GLP-2 dual agonist peptide
  SITE 2: note = MISC_FEATURE - Xaa is Aib
  SITE 17: note = MISC_FEATURE - Lys is L-Lys in which the side chain is conjugated to thesubstituent ([17-carboxy-heptadecanoyl]-isoGlu)

SEQ ID NO: 118 - protein, 33 aa, synthetic construct; identical to SEQ ID NO: 71
  REGION 1..33: note = GLP-1/GLP-2 dual agonist peptide
  SITE 2: note = MISC_FEATURE - Xaa is Aib
  SITE 17: note = MISC_FEATURE - Lys is L-Lys in which the side chain is conjugated to thesubstituent ([17-carboxy-heptadecanoyl]-isoGlu)

SEQ ID NO: 119 - protein, 33 aa, synthetic construct; identical to SEQ ID NO: 72
  REGION 1..33: note = GLP-1/GLP-2 dual agonist peptide
  SITE 2: note = MISC_FEATURE - Xaa is Aib
  SITE 17: note = MISC_FEATURE - Lys is L-Lys in which the side chain is conjugated to thesubstituent ([17-carboxy-heptadecanoyl]-isoGlu)

SEQ ID NO: 120 - protein, 33 aa, synthetic construct; identical to SEQ ID NO: 73
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 121 - protein, 33 aa, synthetic construct; identical to SEQ ID NO: 74
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 122 - protein, 33 aa, synthetic construct; identical to SEQ ID NO: 75
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 123 - protein, 33 aa, synthetic construct; identical to SEQ ID NO: 76
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 124 - protein, 33 aa, synthetic construct; identical to SEQ ID NO: 77
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 125 - protein, 33 aa, synthetic construct; identical to SEQ ID NO: 78
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 126 - protein, 33 aa, synthetic construct; identical to SEQ ID NO: 79
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 127 - protein, 33 aa, synthetic construct; identical to SEQ ID NO: 80
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 128 - protein, 33 aa, synthetic construct; identical to SEQ ID NO: 81
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 129 - protein, 33 aa, synthetic construct; identical to SEQ ID NO: 82
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 130 - protein, 33 aa, synthetic construct; identical to SEQ ID NO: 83
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 131 - protein, 33 aa, synthetic construct; identical to SEQ ID NO: 84
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 132 - protein, 33 aa, synthetic construct; identical to SEQ ID NO: 85
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 133 - protein, 33 aa, synthetic construct; identical to SEQ ID NO: 86
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 134 - protein, 33 aa, synthetic construct; identical to SEQ ID NO: 87
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 135 - protein, 33 aa, synthetic construct; identical to SEQ ID NO: 88
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 136 - protein, 33 aa, synthetic construct; identical to SEQ ID NO: 89
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 137 - protein, 33 aa, synthetic construct; identical to SEQ ID NO: 90
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 138 - protein, 33 aa, synthetic construct; identical to SEQ ID NO: 91
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 139 - protein, 33 aa, synthetic construct; identical to SEQ ID NO: 92
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 140 - protein, 33 aa, synthetic construct; identical to SEQ ID NO: 93
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 141 - protein, 33 aa, synthetic construct; identical to SEQ ID NO: 94
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu)

SEQ ID NO: 142 - protein, 33 aa, synthetic construct; identical to SEQ ID NO: 95
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([hexadecanoyl]-beta-Ala)

SEQ ID NO: 143 - protein, 33 aa, synthetic construct; identical to SEQ ID NO: 96
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-carboxy-heptadecanoyl]-isoGlu-Peg3)

SEQ ID NO: 144 - protein, 33 aa, synthetic construct; identical to SEQ ID NO: 97
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-carboxy-nonadecanoyl]iso-Glu-Peg3-Peg3)

SEQ ID NO: 145 - protein, 33 aa, synthetic construct; identical to SEQ ID NO: 98
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-carboxy-nonadecanoyl]iso-Lys-Peg3-Peg3-Peg3)

SEQ ID NO: 146 - protein, 33 aa, synthetic construct; identical to SEQ ID NO: 99
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent (Octadecanoyl)

SEQ ID NO: 147 - protein, 33 aa, synthetic construct; identical to SEQ ID NO: 100
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent (Hexadecanoyl-isoGlu)

SEQ ID NO: 148 - protein, 39 aa, synthetic construct; identical to SEQ ID NO: 101
  REGION 1..39: note = GLP-1/GLP-2 dual agonist peptide
  SITE 2: note = MISC_FEATURE - Xaa is Aib
  SITE 34..38: note = Lys is D-Lys
  SITE 39: note = MISC_FEATURE - Lys is D-Lys in which the side chain is conjugated to thesubstituent ([17-carboxy-Heptadecanoyl]-isoGlu)

SEQ ID NO: 149 - protein, 39 aa, synthetic construct; identical to SEQ ID NO: 102
  REGION 1..39: note = GLP-1/GLP-2 dual agonist peptide
  SITE 2: note = MISC_FEATURE - Xaa is Aib
  SITE 34..38: note = Lys is D-Lys
  SITE 39: note = MISC_FEATURE - Lys is D-Lys in which the side chain is conjugated to thesubstituent ([17-carboxy-Heptadecanoyl]-isoGlu)

SEQ ID NO: 150 - protein, 39 aa, synthetic construct; identical to SEQ ID NO: 103
  REGION 1..39: note = GLP-1/GLP-2 dual agonist peptide
  SITE 2: note = MISC_FEATURE - Xaa is Aib
  SITE 34..38: note = Lys is D-Lys
  SITE 39: note = MISC_FEATURE - Lys is D-Lys in which the side chain is conjugated to thesubstituent ([17-carboxy-Heptadecanoyl]-isoGlu)

SEQ ID NO: 151 - protein, 33 aa, synthetic construct; identical to SEQ ID NO: 82
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-carboxy-nonadecanoyl]-isoGlu)

SEQ ID NO: 152 - protein, 33 aa, synthetic construct; identical to SEQ ID NO: 104
  REGION 1..33: note = GLP-1/GLP-2 dual agonist peptide
  SITE 2: note = MISC_FEATURE - Xaa is Aib
  SITE 10: note = MISC_FEATURE - Lys is L-Lys in which the side chain is conjugated to thesubstituent ([19-carboxy-nonadecanoyl]iso-Glu-Peg3-Peg3)

SEQ ID NO: 153 - protein, 33 aa, synthetic construct; identical to SEQ ID NO: 68
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-carboxy-nonadecanoyl]iso-Glu-Peg3-Peg3)

SEQ ID NO: 154 - protein, 33 aa, synthetic construct; identical to SEQ ID NO: 105
  REGION 1..33: note = GLP-1/GLP-2 dual agonist peptide
  SITE 2: note = MISC_FEATURE - Xaa is Aib
  SITE 17: note = MISC_FEATURE - Lys is L-Lys in which the side chain is conjugated to thesubstituent ([19-carboxy-nonadecanoyl]iso-Glu-Peg3-Peg3)

SEQ ID NO: 155 - protein, 33 aa, synthetic construct; identical to SEQ ID NO: 106
  REGION 1..33: note = GLP-1/GLP-2 dual agonist peptide
  SITE 2: note = MISC_FEATURE - Xaa is Aib
  SITE 20: note = MISC_FEATURE - Lys is L-Lys in which the side chain is conjugated to thesubstituent ([19-carboxy-nonadecanoyl]iso-Glu-Peg3-Peg3)

SEQ ID NO: 156 - protein, 33 aa, synthetic construct; identical to SEQ ID NO: 107
  REGION 1..33: note = GLP-1/GLP-2 dual agonist peptide
  SITE 2: note = MISC_FEATURE - Xaa is Aib
  SITE 20: note = MISC_FEATURE - Lys is L-Lys in which the side chain is conjugated to thesubstituent ([19-carboxy-nonadecanoyl]iso-Glu-Peg3-Peg3)

SEQ ID NO: 157 - protein, 33 aa, synthetic construct; identical to SEQ ID NO: 80
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-Carboxy-heptadecanoyl]-isoGlu-KEK-Peg3)

SEQ ID NO: 158 - protein, 33 aa, synthetic construct; identical to SEQ ID NO: 80
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-Carboxy-nonadecanoyl]-isoGlu-KEK-Peg3)

SEQ ID NO: 159 - protein, 33 aa, synthetic construct; identical to SEQ ID NO: 83
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([17-Carboxy-heptadecanoyl]-isoGlu-KEK-Peg3)

SEQ ID NO: 160 - protein, 33 aa, synthetic construct; identical to SEQ ID NO: 83
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-Carboxy-nonadecanoyl]-isoGlu-KEK-Peg3)

SEQ ID NO: 161 - protein, 33 aa, synthetic construct; identical to SEQ ID NO: 82
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-Carboxy-nonadecanoyl]-isoGlu-KEK)

SEQ ID NO: 162 - protein, 33 aa, synthetic construct; identical to SEQ ID NO: 82
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-Carboxy-nonadecanoyl]-isoGlu-KEK-Peg3)

SEQ ID NO: 163 - protein, 33 aa, synthetic construct; identical to SEQ ID NO: 104
  REGION 1..33: note = GLP-1/GLP-2 dual agonist peptide
  SITE 2: note = MISC_FEATURE - Xaa is Aib
  SITE 10: note = MISC_FEATURE - Lys is L-Lys in which the side chain is conjugated to thesubstituent ([19-Carboxy-nonadecanoyl]-isoGlu-KEK-Peg3)

SEQ ID NO: 164 - protein, 33 aa, synthetic construct; identical to SEQ ID NO: 108
  REGION 1..33: note = GLP-1/GLP-2 dual agonist peptide
  SITE 2: note = MISC_FEATURE - Xaa is Aib
  SITE 10: note = MISC_FEATURE - Lys is L-Lys in which the side chain is conjugated to thesubstituent ([19-Carboxy-nonadecanoyl]-isoGlu-KEK-Peg3)

SEQ ID NO: 165 - protein, 33 aa, synthetic construct; identical to SEQ ID NO: 104
  REGION 1..33: note = GLP-1/GLP-2 dual agonist peptide
  SITE 2: note = MISC_FEATURE - Xaa is Aib
  SITE 10: note = MISC_FEATURE - Lys is L-Lys in which the side chain is conjugated to thesubstituent ([19-carboxy-nonadecanoyl]iso-Glu-KEK-Peg3-Peg3)

SEQ ID NO: 166 - protein, 33 aa, synthetic construct; identical to SEQ ID NO: 88
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-Carboxy-nonadecanoyl]-isoGlu-KEK-Peg3)

SEQ ID NO: 167 - protein, 33 aa, synthetic construct; identical to SEQ ID NO: 87
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-Carboxy-nonadecanoyl]-isoGlu-KEK-Peg3)

SEQ ID NO: 168 - protein, 33 aa, synthetic construct; identical to SEQ ID NO: 85
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-Carboxy-nonadecanoyl]-isoGlu-KEK-Peg3)

SEQ ID NO: 169 - protein, 33 aa, synthetic construct; identical to SEQ ID NO: 68
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-Carboxy-nonadecanoyl]-isoGlu-KEK-Peg3)

SEQ ID NO: 170 - protein, 33 aa, synthetic construct; identical to SEQ ID NO: 87
  REGION 1..33: note = GLP-1/GLP-2 dual agonist peptide
  SITE 2: note = MISC_FEATURE - Xaa is Aib
  SITE 16: note = MISC_FEATURE - Lys is L-Lys in which the side chain is conjugated to thesubstituent ([19-carboxy-nonadecanoyl]iso-Glu-KEK-Peg3-Peg3)

SEQ ID NO: 171 - protein, 33 aa, synthetic construct; identical to SEQ ID NO: 105
  REGION 1..33: note = GLP-1/GLP-2 dual agonist peptide
  SITE 2: note = MISC_FEATURE - Xaa is Aib
  SITE 17: note = MISC_FEATURE - Lys is L-Lys in which the side chain is conjugated to thesubstituent ([19-Carboxy-nonadecanoyl]-isoGlu-KEK-Peg3)

SEQ ID NO: 172 - protein, 33 aa, synthetic construct; identical to SEQ ID NO: 109
  REGION 1..33: note = GLP-1/GLP-2 dual agonist peptide
  SITE 2: note = MISC_FEATURE - Xaa is Aib
  SITE 17: note = MISC_FEATURE - Lys is L-Lys in which the side chain is conjugated to thesubstituent ([19-Carboxy-nonadecanoyl]-isoGlu-KEK-Peg3)

SEQ ID NO: 173 - protein, 33 aa, synthetic construct; identical to SEQ ID NO: 109
  REGION 1..33: note = GLP-1/GLP-2 dual agonist peptide
  SITE 2: note = MISC_FEATURE - Xaa is Aib
  SITE 17: note = MISC_FEATURE - Lys is L-Lys in which the side chain is conjugated to thesubstituent ([19-carboxy-nonadecanoyl]iso-Glu-KEK-Peg3-Peg3)

SEQ ID NO: 174 - protein, 33 aa, synthetic construct; identical to SEQ ID NO: 106
  REGION 1..33: note = GLP-1/GLP-2 dual agonist peptide
  SITE 2: note = MISC_FEATURE - Xaa is Aib
  SITE 20: note = MISC_FEATURE - Lys is L-Lys in which the side chain is conjugated to thesubstituent ([19-Carboxy-nonadecanoyl]-isoGlu-KEK-Peg3)

SEQ ID NO: 175 - protein, 33 aa, synthetic construct; identical to SEQ ID NO: 107
  REGION 1..33: note = GLP-1/GLP-2 dual agonist peptide
  SITE 2: note = MISC_FEATURE - Xaa is Aib
  SITE 20: note = MISC_FEATURE - Lys is L-Lys in which the side chain is conjugated to thesubstituent ([19-Carboxy-nonadecanoyl]-isoGlu-KEK-Peg3)

SEQ ID NO: 176 - protein, 33 aa, synthetic construct; identical to SEQ ID NO: 106
  REGION 1..33: note = GLP-1/GLP-2 dual agonist peptide
  SITE 2: note = MISC_FEATURE - Xaa is Aib
  SITE 20: note = MISC_FEATURE - Lys is L-Lys in which the side chain is conjugated to thesubstituent ([19-carboxy-nonadecanoyl]iso-Glu-KEK-Peg3-Peg3)

SEQ ID NO: 177 - protein, 33 aa, synthetic construct; identical to SEQ ID NO: 107
  REGION 1..33: note = GLP-1/GLP-2 dual agonist peptide
  SITE 2: note = MISC_FEATURE - Xaa is Aib
  SITE 20: note = MISC_FEATURE - Lys is L-Lys in which the side chain is conjugated to thesubstituent ([19-carboxy-nonadecanoyl]iso-Glu-KEK-Peg3-Peg3)

SEQ ID NO: 178 - protein, 33 aa, synthetic construct; identical to SEQ ID NO: 110
  REGION 1..33: note = GLP-1/GLP-2 dual agonist peptide
  SITE 2: note = MISC_FEATURE - Xaa is Aib
  SITE 20: note = MISC_FEATURE - Lys is L-Lys in which the side chain is conjugated to thesubstituent ([19-Carboxy-nonadecanoyl]-isoGlu-KEK-Peg3)

SEQ ID NO: 179 - protein, 6 aa, synthetic construct
  REGION 1..6: note = Linker sequence
  SITE 1..6: note = D-Lysine
KKKKKK

SEQ ID NO: 180 - protein, 4 aa, synthetic construct
  REGION 1..4: note = Linker sequence
  SITE 4: note = MISC_FEATURE - Glu is isoGlu (isoglutamine)
KEKE

SEQ ID NO: 181 - protein, 4 aa, synthetic construct
  REGION 1..4: note = Linker sequence
  SITE 4: note = MISC_FEATURE - Ala is beta-Ala
KEKA

SEQ ID NO: 182 - protein, 4 aa, synthetic construct
  REGION 1..4: note = Linker sequence
  SITE 4: note = MISC_FEATURE - Lys is isoLys
KEKK

SEQ ID NO: 183 - skipped

SEQ ID NO: 184 - protein, 4 aa, synthetic construct
  REGION 1..4: note = Linker sequence
  SITE 1: note = MISC_FEATURE - Ala is beta-Ala
AKEK

SEQ ID NO: 185 - protein, 4 aa, synthetic construct
  REGION 1..4: note = Linker sequence
  SITE 1: note = MISC_FEATURE - Lys is isoLys
KKEK

SEQ ID NO: 186 - skipped

SEQ ID NO: 187 - protein, 15 aa, synthetic construct
  source 1..15: note = the sequence 'U' can comprise 1 to 15 lysine residues; note = Linker sequence
KKKKKKKKKKKKKKK

SEQ ID NO: 188 - protein, 15 aa, synthetic construct
  source 1..15: note = the sequence 'U' can comprise 1 to 15 lysine residues; note = Linker sequence
  SITE 1..15: note = lys is D-lys (i.e, D-lysine)
KKKKKKKKKKKKKKK

SEQ ID NO: 189 - protein, 15 aa, synthetic construct
  source 1..15: note = Linker sequence
  REGION 1..14: note = the sequence 'U' can contain 1 to 14 lys residues
  VARIANT 15: note = X is a residue of K, k, R, Orn, Dap or Dab in which the sidechain is conjugated to a substituent
KKKKKKKKKKKKKKX

SEQ ID NO: 190 - protein, 33 aa, synthetic construct
  REGION 1..33: note = reference compound A
  SITE 2: note = X is Aib
  SITE 16: note = Lys is L-Lys in which the side chain is conjugated to thesubstituent ([17-carboxy-heptadecanoyl]-isoGlu)
HXDGSFSSELATILDKQAARDFIAWLIQHKITD

SEQ ID NO: 191 - protein, 33 aa, synthetic construct
  REGION 1..33: note = Reference compound B
  SITE 2: note = X is Aib
  SITE 16: note = Lys is L-Lys in which the side chain is conjugated to thesubstituent ([17-carboxy-heptadecanoyl]-isoGlu)
HXEGSFSSELATILDKQAARDFIAWLIQHKITD

(19) United States
(12) Patent Application Publication
(10) Pub. No.: US 2025/0073311 A1
(43) Pub. Date: Mar. 6, 2025

(54) DOSAGE REGIME

(71) Applicant: ZEALAND PHARMA A/S, Soborg (DK)

(72) Inventor: Mikkel Askjær AGERSNAP, Søborg (DK)

(21) Appl. No.: 18/688,026

(22) PCT Filed: Sep. 2, 2022
(86) PCT No.: PCT/EP2022/074420

§ 371 Date: Feb. 29, 2024

(30) Foreign Application Priority Data
Sep. 3, 2021 (EP) 21194879.9
Mar. 4, 2022 (EP) 22160234.5

(51) Int. Cl. A61K 38/26; A61P 3/04; C07K 14/605

(52) U.S. Cl. CPC A61K 38/26; A61P 3/04; C07K 14/605

(57) ABSTRACT

The invention relates to a dosage regime for compounds having agonist activity at the GLP-1 (glucagon-like-peptide 1) and GLP-2 (glucagon-like peptide 2) receptors for use in the treatment of obesity and related conditions.

### SEQUENCE LISTING

The instant application contains a Sequence Listing which has been submitted electronically in XML format and is hereby incorporated by reference in its entirety. Said XML copy, created on Oct. 28, 2024, is named “50412-148001_Sequence_Listing_10_28_24” and is 413,099 bytes in size.

### FIELD OF THE INVENTION

The present invention relates to a therapeutic method using acylated compounds having dual agonist activity at the GLP-1 (glucagon-like-peptide 1) and GLP-2 (glucagon-like peptide 2) receptors. In particular, the invention relates to a dosage regimen for the dual GLP-1/GLP-2 agonist peptides for the regulation of body weight and prophylaxis or treatment of obesity and related conditions.

### BACKGROUND TO THE INVENTION

Obesity is a currently a significant public health issue across much of the developed world and is correlated with the development of several serious conditions, such as cardiovascular disease, type 2 diabetes, sleep apnoea, and certain cancers. The standard treatment for obesity is lifestyle intervention, including the reduction of energy intake and the increase of exercise. However, while such interventions can achieve temporary success, it is often challenging for patients to sustain such lifestyle changes over a long period such that the weight loss achieved is permanent.

GLP-1 is released from the gut in response to food intake and hence acts as a satiety signal, leading to reduced food intake (Madsbad, S., 2014, Diabetes Obes Metab, 16: 9-21). There is evidence to suggest that the effect of GLP-1 may be impaired in obese subjects, suggesting that GLP-1 agonists may have promise the in treatment of obesity. However, a significant drawback of GLP-1 therapy is that a significant proportion of patients taking known GLP-1 agonists suffer from side effects of nausea and vomiting (Filippatos et al, 2014/15, Rev Diabet Stud., 11(3): 202-230). These side-effects generally require the dose of the GLP-1 agonist to be gradually escalated from a low starting dose in order to minimize such side effects. Indeed, recent clinical trial data for the GLP-1 agonist Semaglutide shows that nausea and vomiting were commonly observed in patients when administered even when initially administering low doses of the drug (Wilding et al, 2021, N Engl J Med; 384:989-1002). These side-effects are undesirable in that they are liable to reduce patient compliance with treatment.

There is therefore an ongoing need for therapeutics with GLP-1 agonist activity that are effective in the treatment of obesity and related conditions while not exhibiting the expected side effects of nausea and vomiting upon administration.

WO 2018/104561 discloses peptides having dual GLP-1 and GLP-2 agonist activity and proposes medical uses thereof. However, specific dosage regimes for the treatment of obesity and related conditions are not disclosed.

It has now surprisingly been found that the administration of certain peptides having dual GLP-1 and GLP-2 agonist activity at certain dosages results in decreased appetite being observed in patients, without giving rise to the expected side effects of nausea and vomiting. As described herein, effects on appetite reduction with the dual GLP-1 and GLP-2 agonist may occur before (i.e. at lower doses than) nausea and vomiting. This is advantageous over known GLP-1 agonist treatments where the gastrointestinal adverse events (nausea and vomiting) occur before (i.e. at lower doses than) the decreased satiety. This suggests that the dual GLP-1 and GLP-2 agonist may have a better safety profile with regards to gastrointestinal adverse events in indications where appetite reduction is desired. Thus the dosage regime of the invention represents a significant advance on known GLP-1 agonist treatments for obesity.

### SUMMARY OF THE INVENTION

Broadly, the present invention relates to compounds which have agonist activity at the GLP-1 (glucagon-like peptide 1) and GLP-2 (glucagon-like peptide 2) receptors, e.g. as assessed in in vitro potency assays, for use in a method of reducing or inhibiting weight gain, reducing food intake, reducing appetite, promoting weight loss, or treating obesity, morbid obesity, obesity-linked gallbladder disease, or obesity-induced sleep apnea. Such compounds are referred to in this specification as “GLP-1/GLP-2 dual agonists”, or simply “dual agonists”. Thus, the compounds according to the present invention have activities of both GLP-1 (7-36) and GLP-2 (1-33).

In a first aspect there is provided a GLP-1/GLP-2 dual agonist represented by the formula:

R¹—X*—U—R²
wherein:
R¹ is hydrogen (Hy), C₁₋₄ alkyl (e.g. methyl), acetyl, formyl, benzoyl or trifluoroacetyl;
R² is NH₂ or OH;
X* is a peptide of formula I:
H-X2-EG-X5-F-X7-X8-E-X10-X11-TIL-X15-X16-X17-A-X19-X20-X21-FI-X24-WL-X27-X28-X29-KIT-X33 (1) (SEQ ID NO 1)
wherein:
X2 is Aib or G
X5 is T or S;
X7 is T or S;
X8 is S, E or D;
X10 is L, M, V or Ψ;
X11 is A, N or S;
X15 is D or E;
X16 is G, E, A or Ψ;
X17 is Q, E, K, L or Ψ;
X19 is A, V or S;
X20 is R, K or Ψ;
X21 is D, L or E;
X24 is A, N or S;
X27 is 1, Q, K, H or Y;
X28 is Q, E, A, H, Y, L, K, R or S;
X29 is H, Y, K or Q;
X33 is D or E;
U is absent or a sequence of 1-15 residues each independently selected from K, k, E, A, T, I, L and Ψ;
the molecule contains one and only one Ψ, wherein Ψ is a residue of K, k, R, Orn, Dap or Dab in which the side chain is conjugated to a substituent having the formula Z¹— or Z¹—Z²—, wherein
Z¹— is CH₃—(CH₂)₁₀₋₂₂—(CO)— or HOOC—(CH₂)₁₀₋₂₂—(CO)—; and
Z²— is selected from —Z^{S1}—, —Z^{S1}—Z^{S2}—, —Z^{S2}—Z^{S1}, —Z^{S2}—, —Z^{S3}—, —Z^{S1} Z^{S3}—, —Z^{S2} Z^{S3}—, —Z^{S3} Z^{S1}—, —Z^{S3} Z^{S2} Z^{S1}Z^{S2} Z^{S3}—, —Z^{S1}Z^{S3} Z^{S2}—, —Z^{S2}Z^{S1}Z^{S3}—, —Z^{S2} Z^{S3} Z^{S1}—, —Z^{S3} Z^{S1}Z^{S2}—, —Z^{S3} Z^{S2} Z^{S1}—, —Z^{S2} Z^{S3} Z^{S2}—, wherein
Z^{S1} is isoGlu, β-Ala, isoLys, or Ψ-aminobutanoyl;
Z^{S2} is -(Peg3)m- where m is 1, 2, or 3; and
—Z^{S3}— is a peptide sequence of 1-6 amino acid units independently selected from the group consisting of A, L, S, T, Y, Q, D, E, K, k, R, H, F and G;
and wherein at least one of X5 and X7 is T;
or a pharmaceutically acceptable salt or solvate thereof;
for use in a method of reducing or inhibiting weight gain, reducing food intake, reducing appetite, promoting weight loss, or treating obesity, morbid obesity, obesity-linked gallbladder disease, or obesity-induced sleep apnea;
wherein the method comprises administering the dual agonist to the patient at a dose of about 0.1 mg to about 10.0 mg.

The various amino acid positions in peptide X* of the formulae provided here are numbered according to their linear position from N- to C-terminus in the amino acid chain.

In the present context, β-Ala and 3-Aminopropanoyl are used interchangeably.

Dual agonists having aspartic acid (Asp, D) at position 3 and glycine (Gly) in position 4 can be very potent agonists at the GLP-1 and GLP-2 receptors. However, this combination of substitutions results in compounds which are unstable and may not be suitable for long term storage in aqueous solution. Without wishing to be bound by theory, it is believed that the Asp at position 3 may isomerise to iso-Asp via a cyclic intermediate formed between the carboxylic acid functional group of its side chain and the backbone nitrogen atom of the residue at position 4.

It has now been found that molecules having glutamic acid (Glu, E) at position 3 instead of Asp are much less susceptible to such reactions and hence may be considerably more stable when stored in aqueous solution. However, replacement of Asp with Glu at position 3 in molecules having a lipophilic substituent in the middle portion of the peptide (e.g. at or near to positions 16 and 17) tends to reduce the potency at one or both of the GLP-2 receptor and the GLP-1 receptor, even though Glu is present at position 3 of the native GLP-1 molecule.

Simultaneously incorporating a Thr residue at one or both of positions 5 and 7 appears to compensate for some or all of the lost potency. It is believed that further improvements in potency are also provided by incorporation of His (H), Tyr (Y), Lys (K) or Gln (Q) at position 29 instead of the Gly (G) and Thr (T) residues present in wild type human GLP-1 and 2 respectively.

In some embodiments of formula I:
X2 is Aib or G
X5 is T or S;
X7 is T or S;
X8 is S;
X10 is L or Ψ);
X11 is A or S;
X15 is D or E;
X16 is G, E, A or Ψ;
X17 is Q, E, K, L or Ψ;
X19 is A or S;
X20 is R or Ψ;
X21 is D, L or E;
X24 is A;
X27 is I, Q, K, or Y;
X28 is Q, E, A, H, Y, L, K, R or S;
X29 is H, Y or Q; and
X33 is D or E.

Where Ψ is not at X16 or X17, it may be desirable that X16 is E and X17 is Q.

In some embodiments, X11 is A and X15 is D. In other embodiments, X11 is S and X15 is E. In further embodiments, X11 is A and X15 is E.

In some embodiments, X27 is I.

In some embodiments, X29 is H. In certain of these embodiments, X28 is A and X29 is H, or X28 is E and X29 is H.

In some embodiments, X29 is Q and optionally X27 is Q.

In some embodiments, the residues at X27-X29 have a sequence selected from:
IQH;
IEH
IAH;
IHH;
IYH;
ILH;
IKH;
IRH;
ISH;
QQH;
YQH;
KQH;
IQQ;
IQY;
IQT; and
IAY.

In some embodiments, X* is a peptide of formula II:
H-X2-EG-X5-F-X7-SELATILD-X16-X17-AAR-X21-FIAWLI-X28-X29-KITD (II) (SEQ ID NO 2)
wherein:
X2 is Aib or G
X5 is T or S;
X7 is T or S;
X16 is G or Ψ;
X17 is Q, E, K, L or Ψ;
X21 is D or L;
X28 is Q, E, A, H, Y, L, K, R or S;
X29 is H, Y or Q;

In some embodiments of Formula I or Formula II, X16 is Ψ and X17 is Q, E, K or L. For example, X17 may be Q, or X17 may be selected from E, K and L. In other embodiments, X16 is G and X17 is 4.

It may be desirable that X21 is D.

X28 may be selected from Q, E and A, e.g. it may be Q or E. In some residue combinations, Q may be preferred. In others, E may be preferred, including but not limited to when X16 is G and X17 is Ψ. Alternatively, X28 may be selected from A, H, Y, L, K, R and S.

X* may be a peptide of formula III:
H[Aib]EG-X5-F-X7-SE-X10-ATILD-X16-X17-AA-X20-X21-FIAWLI-X28-X29-KITD (Ill) (SEQ ID NO 3)
wherein:
X5 is T or S;
X7 is T or S;
X10 is L or Ψ;
X16 is G, E, A or Ψ;
X17 is Q, E, K, L or Ψ;
X20 is R or Ψ;
X21 is D or L;
X28 is E, A or Q;
X29 is H, Y or Q;
and at least one of X5 and X7 is T.

X* may be a peptide of formula IV:
H[Aib]EG-X5-F-X7-SELATILD-X16-X17-AAR-X21-FIAWLI-X28-X29-KITD (IV) (SEQ ID NO Ψ
wherein:
X5 is T or S;
X7 is T or S;
X16 is G or Ψ;
X17 is E, K, L or Ψ;
X21 is D or L;
X28 is E or A;
X29 is H, Y or Q;
and at least one of X5 and X7 is T.

In some embodiments of any of formulae I to IV, X16 is Ψ and X17 is E, K or L.

In other embodiments of formulae I to IV, X16 is G and X17 is Ψ.

In either case, the following combinations of residues may also be included:
X21 is D and X28 is E;
X21 is D and X28 is A;
X21 is L and X28 is E;
X21 is L and X28 is A.

X* may be a peptide of formula V:
H[Aib]EG-X5-F-X7-SELATILD-Ψ-QAARDFIAWLI-X28-X29-KITD (V) (SEQ ID NO 5)
wherein
X5 is T or S;
X7 is T or S;
X28 is Q, E, A, H, Y, L, K, R or S, e.g. Q, E, A, H, Y or L;
X29 is H, Y or Q;
and at least one of X5 and X7 is T.

In some embodiments of formula III, X28 is Q or E. In some embodiments of formula III, X28 is Q. In other embodiments, X28 is A, H, Y, L, K, R or S, e.g. A, H, Y or L.

In any of the formulae or embodiments described above, the dual agonist contains one of the following combinations of residues:
X5 is S and X7 is T;
X5 is T and X7 is S;
X5 is T and X7 is T.

It may be preferred that X5 is S and X7 is T, or X5 is T and X7 is T.

In any of the formulae or embodiments described above, it may be desirable that X29 is H.

In some embodiments, Ψ is a Lys residue whose side chain is conjugated to the substituent Z¹-or Z¹—Z²—.

In some embodiments, Z¹—, alone or in combination with —Z²—, is dodecanoyl, tetradecanoyl, hexadecanoyl, octadecanoyl or eicosanoyl.

In some embodiments, Z¹—, alone or in combination with —Z²—, is:
13-carboxytridecanoyl, i.e. HOOC—(CH₂)₁₂—(CO)—;
15-carboxypentadecanoyl, i.e. HOOC—(CH₂)₁₄—(CO)—;
17-carboxyheptadecanoyl, i.e. HOOC—(CH₂)₁₆—(CO)—;
19-carboxynonadecanoyl, i.e. HOOC—(CH₂)₁₈—(CO)—; or
21-carboxyheneicosanoyl, i.e. HOOC—(CH₂)₂₀—(CO)—.

In some embodiments Z² is absent.

In some embodiments, Z² comprises Z^{S1} alone or in combination with Z^{S2} and/or Z^{S3}.

In such embodiments:
—Z^{S1}— is isoGlu, β-Ala, isoLys, or 4-aminobutanoyl;
—Z^{S2}—, when present, is -(Peg3)ₘ- where m is 1, 2, or 3; and
—Z^{S3}— is a peptide sequence of 1-6 amino acid units independently selected from the group consisting of A, L, S, T, Y, Q, D, E, K, k, R, H, F and G, such as the peptide sequence KEK.

Z² may have the formula —Z^{S1}—Z^{S3}—Z^{S2}—, where Z^{S1} is bonded to Z¹ and Z^{S2} is bonded to the side chain of the amino acid component of Ψ.

Thus, in some embodiments, —Z²— is:
isoGlu(Peg 3)₀₋₃;
β-Ala(Peg 3)₀₋₃;
isoLys(Peg3)₀₋₃; or
4-aminobutanoyl(Peg3)₀₋₃.

In further embodiments, —Z²— is:
isoGlu-KEK-(Peg3)₀₋₃ (SEQ ID NO 6).

Specific examples of the substituent Z¹—Z²— are set out below. In some embodiments, Z¹—Z²— is
[17-carboxy-heptadecanoyl]-isoGlu. For example, Ψ may be K([17-carboxy-heptadecanoyl]-isoGlu). In some embodiments, Z¹—Z²— is:
[17-Carboxy-heptadecanoyl]-isoGlu-KEK-Peg3-;
[17-carboxy-heptadecanoyl]-isoGlu-Peg3-;
[19-Carboxy-nonadecanoyl]-isoGlu-;
[19-Carboxy-nonadecanoyl]-isoGlu-KEK-;
[19-Carboxy-nonadecanoyl]-isoGlu-KEK-Peg3-;
[19-carboxy-nonadecanoyl]-isoGlu-KEK-Peg3-Peg3-;
[19-carboxy-nonadecanoyl]-isoGlu-Peg3-Peg3-;
[19-carboxy-nonadecanoyl]-isoLys-Peg3-Peg3-Peg3-;
[Hexadecanoyl]-βAla-;
[Hexadecanoyl]-isoGlu-; or
Octadecanoyl-.

For example, Ψ may be:
K([17-Carboxy-heptadecanoyl]-isoGlu-KEK-Peg3);
K([17-carboxy-heptadecanoyl]-isoGlu-Peg3);
K([19-Carboxy-nonadecanoyl]-isoGlu);
K([19-Carboxy-nonadecanoyl]-isoGlu-KEK);
K([19-Carboxy-nonadecanoyl]-isoGlu-KEK-Peg3);
K([19-carboxy-nonadecanoyl]-isoGlu-KEK-Peg3-Peg3);
K([19-carboxy-nonadecanoyl]-isoGlu-Peg3-Peg3);
K([19-carboxy-nonadecanoyl]-isoLys-Peg3-Peg3-Peg3);
K([Hexadecanoyl]-βAla-;
K([Hexadecanoyl]-isoGlu); or
K(Octadecanoyl).

When present, U represents a peptide sequence of 1-15 residues each independently selected from K (i.e. L-lysine), k (i.e. D-lysine) E (Glu), A (Ala), T (Thr), I (lie), L (Leu) and Ψ. For example, U may be 1-10 amino acids in length, 1-7 amino acids in length, 3-7 amino acids in length, 1-6 amino acids in length, or 3-6 amino acids in length.

Typically, U includes at least one charged amino acid (K, k or E) and preferably two or more charged amino acids. In some embodiments it includes at least 2 positively charged amino acids (K or k), or at least 1 positively charged amino acid (K or k) and at least one negatively charged amino acid (E). In some embodiments, all amino acid residues of U (except for Ψ, if present) are charged. For example, U may be a chain of alternately positively and negatively charged amino acids.

In certain embodiments, U comprises residues selected only from K, k, E and Ψ.

In certain embodiments, U comprises residues selected only from K, k, and Ψ.

When U comprises only lysine residues (whether K or k), all residues may have an L-configuration or all may have a D-configuration. Examples include K₁₋₁₅, K₁₋₁₀ and K₁₋₇, e.g., K₃, K₄, K₅, K₆ and K₇, especially K₅ and K₆. Further examples include k₁₀₋₁₅, k₁₀₋₁₀ and k₁₀₋₇, e.g. k₃, k₄, k₅, k₆ and k₇, especially k₅ and k₆.

Further examples of peptide sequences U include KEK, EKEKEK (SEQ ID NO 7), EkEkEk (SEQ ID NO 8), AKAAEK (SEQ ID NO 9), AKEKEK (SEQ ID NO 10) and ATILEK (SEQ ID NO 11).

In any case, one of those residues may be exchanged for Ψ. Where the sequence U contains a residue Ψ, it may be desirable that the C-terminal residue of U is 4. Thus, further examples of sequences U include K₁₋₁₄-Ψ, K₁₋₉-Ψ and K₁₋₆-Ψ, e.g., K₂-Ψ, K₃-Ψ, K₄-Ψ, K₅-Ψ and K₆-Ψ, especially K₄-Ψ and K₅-Ψ. Yet further examples include k₁₋₁₄-Ψ, k₁₋₉-Ψ, and k₁₋₆-), e.g. k₂-Ψ, k₃-Ψ, k₄-Ψ, k₅-Ψ and k₆-Ψ especially k₄-Ψ and k₅-Ψ. Yet further examples include KE4P, EKEKEΨ (SEQ ID NO 12), EkEkEΨ (SEQ ID NO 13) AKAAEΨ (SEQ ID NO 14), AKEKEΨ (SEQ ID NO 15) and ATILEΨ (SEQ ID NO 16).

In some embodiments, U is absent.

In some embodiments, R¹ is Hy and/or R² is OH.

(SEQ ID NO 17)
H[Aib]EGTFSSELATILDΨEAARDFIAWLIEHKITD;
(SEQ ID NO 18)
H[Aib]EGSFTSELATILDΨEAARDFIAWLIEHKITD;
(SEQ ID NO 19)
H[Aib]EGTFTSELATILDΨPEAARDFIAWLIEHKITD;
(SEQ ID NO 20)
H[Aib]EGTFSSELATILDΨKAARDFIAWLIEHKITD;
(SEQ ID NO 21)
H[Aib]EGSFTSELATILDΨKAARDFIAWLIEHKITD;
(SEQ ID NO 22)
H[Aib]EGTFTSELATILDΨKAARDFIAWLIEHKITD;
(SEQ ID NO 23)
H[Aib]EGTFSSELATILDGΨAARDFIAWLIEHKITD;
(SEQ ID NO 24)
H[Aib]EGSFTSELATILDGΨAARDFIAWLIEHKITD;
(SEQ ID NO 25)
H[Aib]EGTFTSELATILDGΨAARDFIAWLIEHKITD;
(SEQ ID NO 26)
H[Aib]EGTFSSELATILDΨLAARDFIAWLIEHKITD;
(SEQ ID NO 27)
H[Aib]EGSFTSELATILDΨLAARDFIAWLIEHKITD;
(SEQ ID NO 28)
H[Aib]EGTFTSELATILDΨLAARDFIAWLIEHKITD;
(SEQ ID NO 29)
H[Aib]EGTFSSELATILDΨLAARDFIAWLIAHKITD;
(SEQ ID NO 30)
H[Aib]EGSFTSELATILDΨLAARDFIAWLIAHKITD;
(SEQ ID NO 31)
H[Aib]EGTFTSELATILDΨLAARDFIAWLIAHKITD;
(SEQ ID NO 32)
H[Aib]EGTFTSELATILDΨEAARLFIAWLIEHKITD;
(SEQ ID NO 33)
H[Aib]EGTFSSELATILDΨQAARDFIAWLIQHKITD;
(SEQ ID NO 34)
H[Aib]EGSFTSELATILDΨQAARDFIAWLIQHKITD;
(SEQ ID NO 35)
H[Aib]EGTFTSELATILDΨQAARDFIAWLIQHKITD;
(SEQ ID NO 36)
H[Aib]EGTFSSELATILDΨQAARDFIAWLIEHKITD;
(SEQ ID NO 37)
H[Aib]EGTFSSELATILDΨQAARDFIAWLIAHKITD;
(SEQ ID NO 38)
H[Aib]EGSFTSELATILDΨQAARDFIAWLIAHKITD;
(SEQ ID NO 39)
H[Aib]EGTFTSELATILDΨQAARDFIAWLIAHKITD;
(SEQ ID NO 40)
H[Aib]EGSFTSELATILDΨQAARDFIAWLIEHKITD;
(SEQ ID NO 41)
H[Aib]EGTFTSELATILDΨQAARDFIAWLIEHKITD;
(SEQ ID NO 42)
H[Aib]EGSFTSELATILDΨQAARDFIAWLIHHKITD;
(SEQ ID NO 43)
H[Aib]EGSFTSELATILDΨQAARDFIAWLIYHKITD;
(SEQ ID NO 44)
H[Aib]EGSFTSELATILDΨQAARDFIAWLILHKITD;
(SEQ ID NO 45)
H[Aib]EGSFTSELATILDΨQAARDFIAWLIKHKITD;
(SEQ ID NO 46)
H[Aib]EGSFTSELATILDΨQAARDFIAWLIRHKITD;
(SEQ ID NO 47)
H[Aib]EGSFTSELATILDΨQAARDFIAWLISHKITD;
(SEQ ID NO 48)
H[Aib]EGSFTSELATILDΨQAARDFIAWLQQHKITD;
(SEQ ID NO 49)
H[Aib]EGSFTSELATILDΨQAARDFIAWLYQHKITD;
(SEQ ID NO 50)
H[Aib]EGSFTSELATILDΨQAARDFIAWLKQHKITD;
(SEQ ID NO 51)
H[Aib]EGSFTSELATILDΨQAARDFIAWLIQQKITD;
(SEQ ID NO 52)
H[Aib]EGSFTSELATILDΨQAARDFIAWLIQYKITD;
(SEQ ID NO 53)
H[Aib]EGTFSSELSTILEΨQASREFIAWLIAYKITE;
(SEQ ID NO 54)
H[Aib]EGTFSSELATILDEQAARDFIAWLIAHKITDkkkkkΨ;
(SEQ ID NO 55)
H[Aib]EGTFTSELATILDEQAARDFIAWLIAHKITDkkkkkΨ;
(SEQ ID NO 56)
H[Aib]EGSFTSELATILDEQAARDFIAWLIEHKITDkkkkkΨ;
(SEQ ID NO 57)
H[Aib]EGSFTSEΨATILDEQAARDFIAWLIEHKITD;
(SEQ ID NO 58)
H[Aib]EGSFTSELATILEGΨAARDFIAWLIEHKITD;
(SEQ ID NO 59)
H[Aib]EGSFTSELATILDEQAAΨDFIAWLIEHKITD;
(SEQ ID NO 60)
H[Aib]EGTFTSELATILDEQAAΨDFIAWLIEHKITD;
(SEQ ID NO 61)
H[Aib]EGTFTSEΨATILDEQAARDFIAWLIEHKITD;
(SEQ ID NO 62)
H[Aib]EGSFTSELATILDAΨAARDFIAWLIEHKITD;
or
(SEQ ID NO 63)
H[Aib]EGSFTSELATILDAKAAΨDFIAWLIEHKITD.

The peptide X* or the peptide X*-U may have the sequence:

(SEQ ID NO 64)
H[Aib]EGTFSSELATILD[K*]EAARDFIAWLIEHKITD;
(SEQ ID NO 65)
H[Aib]EGSFTSELATILD[K*]EAARDFIAWLIEHKITD;
(SEQ ID NO 66)
H[Aib]EGTFTSELATILD[K*]EAARDFIAWLIEHKITD;
(SEQ ID NO 67)
H[Aib]EGTFSSELATILD[K*]KAARDFIAWLIEHKITD;
(SEQ ID NO 68)
H[Aib]EGSFTSELATILD[K*]KAARDFIAWLIEHKITD;
(SEQ ID NO 69)
H[Aib]EGTFTSELATILD[K*]KAARDFIAWLIEHKITD;
(SEQ ID NO 70)
H[Aib]EGTFSSELATILDG[K*]AARDFIAWLIEHKITD;
(SEQ ID NO 71)
H[Aib]EGSFTSELATILDG[K*]AARDFIAWLIEHKITD;
(SEQ ID NO 72)
H[Aib]EGTFTSELATILDG[K*]AARDFIAWLIEHKITD;
(SEQ ID NO 73)
H[Aib]EGTFSSELATILD[K*]LAARDFIAWLIEHKITD;
(SEQ ID NO 74)
H[Aib]EGSFTSELATILD[K*]LAARDFIAWLIEHKITD;
(SEQ ID NO 75)
H[Aib]EGTFTSELATILD[K*]LAARDFIAWLIEHKITD;
(SEQ ID NO 76)
H[Aib]EGTFSSELATILD[K*]LAARDFIAWLIAHKITD;
(SEQ ID NO 77)
H[Aib]EGSFTSELATILD[K*]LAARDFIAWLIAHKITD;
(SEQ ID NO 78)
H[Aib]EGTFTSELATILD[K*]LAARDFIAWLIAHKITD;
(SEQ ID NO 79)
H[Aib]EGTFTSELATILD[K*]EAARLFIAWLIEHKITD;
(SEQ ID NO 80)
H[Aib]EGTFSSELATILD[K*]QAARDFIAWLIQHKITD;
(SEQ ID NO 81)
H[Aib]EGSFTSELATILD[K*]QAARDFIAWLIQHKITD;
(SEQ ID NO 82)
H[Aib]EGTFTSELATILD[K*]QAARDFIAWLIQHKITD;
(SEQ ID NO 83)
H[Aib]EGTFSSELATILD[K*]QAARDFIAWLIEHKITD;
(SEQ ID NO 84)
H[Aib]EGTFSSELATILD[K*]QAARDFIAWLIAHKITD;
(SEQ ID NO 85)
H[Aib]EGSFTSELATILD[K*]QAARDFIAWLIAHKITD;
(SEQ ID NO 86)
H[Aib]EGTFTSELATILD[K*]QAARDFIAWLIAHKITD;
(SEQ ID NO 87)
H[Aib]EGSFTSELATILD[K*]QAARDFIAWLIEHKITD;
(SEQ ID NO 88)
H[Aib]EGTFTSELATILD[K*]QAARDFIAWLIEHKITD;
(SEQ ID NO 89)
H[Aib]EGSFTSELATILD[K*]QAARDFIAWLIHHKITD;
(SEQ ID NO 90)
H[Aib]EGSFTSELATILD[K*]QAARDFIAWLIYHKITD;
(SEQ ID NO 91)
H[Aib]EGSFTSELATILD[K*]QAARDFIAWLILHKITD;
(SEQ ID NO 92)
H[Aib]EGSFTSELATILD[K*]QAARDFIAWLIKHKITD;
(SEQ ID NO 93)
H[Aib]EGSFTSELATILD[K*]QAARDFIAWLIRHKITD;
(SEQ ID NO 94)
H[Aib]EGSFTSELATILD[K*]QAARDFIAWLISHKITD;
(SEQ ID NO 95)
H[Aib]EGSFTSELATILD[K*]QAARDFIAWLQQHKITD;
(SEQ ID NO 96)
H[Aib]EGSFTSELATILD[K*]QAARDFIAWLYQHKITD;
(SEQ ID NO 97)
H[Aib]EGSFTSELATILD[K*]QAARDFIAWLKQHKITD;
(SEQ ID NO 98)
H[Aib]EGSFTSELATILD[K*]QAARDFIAWLIQQKITD;
(SEQ ID NO 99)
H[Aib]EGSFTSELATILD[K*]QAARDFIAWLIQYKITD;
(SEQ ID NO 100)
H[Aib]EGTFSSELSTILE[K*]QASREFIAWLIAYKITE;
(SEQ ID NO 101)
H[Aib]EGTFSSELATILDEQAARDFIAWLIAHKITDkkkkk[k*];
(SEQ ID NO 102)
H[Aib]EGTFTSELATILDEQAARDFIAWLIAHKITDkkkkk[k*];
(SEQ ID NO 103)
H[Aib]EGSFTSELATILDEQAARDFIAWLIEHKITDkkkkk[k*];
(SEQ ID NO 104)
H[Aib]EGSFTSE[K*]ATILDEQAARDFIAWLIEHKITD;
(SEQ ID NO 105)
H[Aib]EGSFTSELATILEG[K*]AARDFIAWLIEHKITD;
(SEQ ID NO 106)
H[Aib]EGSFTSELATILDEQAA[K*]DFIAWLIEHKITD;
(SEQ ID NO 107)
H[Aib]EGTFTSELATILDEQAA[K*]DFIAWLIEHKITD;
(SEQ ID NO 108)
H[Aib]EGTFTSE[K*]ATILDEQAARDFIAWLIEHKITD;
(SEQ ID NO 109)
H[Aib]EGSFTSELATILDA[K*]AARDFIAWLIEHKITD;
or
(SEQ ID NO 110)
H[Aib]EGSFTSELATILDAKAA[K*]DFIAWLIEHKITD;

wherein K* or k* indicates an L or D lysine residue respectively in which the side chain is conjugated to the substituent Z¹— or Z¹Z²—.

For example, the peptide X* or the peptide X*-U may have the sequence:

(SEQ ID NO 111)
H[Aib]EGTFSSELATILD[K([17-carboxy-heptadecanoyl]-isoGlu)]EAARDFIAWLIEHKITD;
(SEQ ID NO 112)
H[Aib]EGSFTSELATILD[K([17-carboxy-heptadecanoyl]-isoGlu)]EAARDFIAWLIEHKITD;
(SEQ ID NO 113)
H[Aib]EGTFTSELATILD[K([17-carboxy-heptadecanoyl]-isoGlu)]EAARDFIAWLIEHKITD;
(SEQ ID NO 114)
H[Aib]EGTFSSELATILD[K([17-carboxy-heptadecanoyl]-isoGlu)]KAARDFIAWLIEHKITD;
(SEQ ID NO 115)
H[Aib]EGSFTSELATILD[K([17-carboxy-heptadecanoyl]-isoGlu)]KAARDFIAWLIEHKITD;
(SEQ ID NO 116)
H[Aib]EGTFTSELATILD[K([17-carboxy-heptadecanoyl]-isoGlu)]KAARDFIAWLIEHKITD;
(SEQ ID NO 117)
H[Aib]EGTFSSELATILDG[K([17-carboxy-heptadecanoyl]-isoGlu)]AARDFIAWLIEHKITD;
(SEQ ID NO 118)
H[Aib]EGSFTSELATILDG[K([17-carboxy-heptadecanoyl]-isoGlu)]AARDFIAWLIEHKITD;
(SEQ ID NO 119)
H[Aib]EGTFTSELATILDG[K([17-carboxy-heptadecanoyl]-isoGlu)]AARDFIAWLIEHKITD;
(SEQ ID NO 120)
H[Aib]EGTFSSELATILD[K([17-carboxy-heptadecanoyl]-isoGlu)]LAARDFIAWLIEHKITD;
(SEQ ID NO 121)
H[Aib]EGSFTSELATILD[K([17-carboxy-heptadecanoyl]-isoGlu)]LAARDFIAWLIEHKITD;
(SEQ ID NO 122)
H[Aib]EGTFTSELATILD[K([17-carboxy-heptadecanoyl]-isoGlu)]LAARDFIAWLIEHKITD;
(SEQ ID NO 123)
H[Aib]EGTFSSELATILD[K([17-carboxy-heptadecanoyl]-isoGlu)]LAARDFIAWLIAHKITD;
(SEQ ID NO 124)
H[Aib]EGSFTSELATILD[K([17-carboxy-heptadecanoyl]-isoGlu)]LAARDFIAWLIAHKITD;
(SEQ ID NO 125)
H[Aib]EGTFTSELATILD[K([17-carboxy-heptadecanoyl]-isoGlu)]LAARDFIAWLIAHKITD;
(SEQ ID NO 126)
H[Aib]EGTFTSELATILD[K([17-carboxy-heptadecanoyl]-isoGlu)]EAARLFIAWLIEHKITD;
(SEQ ID NO 127)
H[Aib]EGTFSSELATILD[K([17-carboxy-heptadecanoyl]-isoGlu)]QAARDFIAWLIQHKITD;
(SEQ ID NO 128)
H[Aib]EGSFTSELATILD[K([17-carboxy-heptadecanoyl]-isoGlu)]QAARDFIAWLIQHKITD;
(SEQ ID NO 129)
H[Aib]EGTFTSELATILD[K([17-carboxy-heptadecanoyl]-isoGlu)]QAARDFIAWLIQHKITD;
(SEQ ID NO 130)
H[Aib]EGTFSSELATILD[K([17-carboxy-heptadecanoyl]-isoGlu)]QAARDFIAWLIEHKITD;
(SEQ ID NO 131)
H[Aib]EGTFSSELATILD[K([17-carboxy-heptadecanoyl]-isoGlu)]QAARDFIAWLIAHKITD;
(SEQ ID NO 132)
H[Aib]EGSFTSELATILD[K([17-carboxy-heptadecanoyl]-isoGlu)]QAARDFIAWLIAHKITD;
(SEQ ID NO 133)
H[Aib]EGTFTSELATILD[K([17-carboxy-heptadecanoyl]-isoGlu)]QAARDFIAWLIAHKITD;
(SEQ ID NO 134)
H[Aib]EGSFTSELATILD[K([17-carboxy-heptadecanoyl]-isoGlu)]QAARDFIAWLIEHKITD;
(SEQ ID NO 135)
H[Aib]EGTFTSELATILD[K([17-carboxy-heptadecanoyl]-isoGlu)]QAARDFIAWLIEHKITD;
(SEQ ID NO 136)
H[Aib]EGSFTSELATILD[K([17-carboxy-heptadecanoyl]-isoGlu)]QAARDFIAWLIHHKITD;
(SEQ ID NO 137)
H[Aib]EGSFTSELATILD[K([17-carboxy-heptadecanoyl]-isoGlu)]QAARDFIAWLIYHKITD;
(SEQ ID NO 138)
H[Aib]EGSFTSELATILD[K([17-carboxy-heptadecanoyl]-isoGlu)]QAARDFIAWLILHKITD;
(SEQ ID NO 139)
H[Aib]EGSFTSELATILD[K([17-carboxy-heptadecanoyl]-isoGlu)]QAARDFIAWLIKHKITD;
(SEQ ID NO 140)
H[Aib]EGSFTSELATILD[K([17-carboxy-heptadecanoyl]-isoGlu)]QAARDFIAWLIRHKITD;
(SEQ ID NO 141)
H[Aib]EGSFTSELATILD[K([17-carboxy-heptadecanoyl]-isoGlu)]QAARDFIAWLISHKITD;
(SEQ ID NO 142)
H[Aib]EGSFTSELATILD[K([Hexadecanoyl]-βAla)]QAARDFIAWLQQHKITD;
(SEQ ID NO 143)
H[Aib]EGSFTSELATILD[K([17-carboxy-heptadecanoyl]iso-Glu-
Peg3)]QAARDFIAWLYQHKITD;
(SEQ ID NO 144)
H[Aib]EGSFTSELATILD[K([19-carboxy-nonadecanoyl]iso-Glu-Peg3-
Peg3)]QAARDFIAWLKQHKITD;
(SEQ ID NO 145)
H[Aib]EGSFTSELATILD[K([19-carboxy-nonadecanoyl]iso-Lys-Peg3-Peg3-
Peg3)]QAARDFIAWLIQQKITD;
(SEQ ID NO 146)
H[Aib]EGSFTSELATILD[K(Octadecanoyl)]QAARDFIAWLIQYKITD;
(SEQ ID NO 147)
H[Aib]EGTFSSELSTILE[K(Hexadecanoyl-isoGlu)]QASREFIAWLIAYKITE;
(SEQ ID NO 148)
H[Aib]EGTFSSELATILDEQAARDFIAWLIAHKITDkkkkkk([17-carboxy-Heptadecanoyl]-
isoGlu)];
(SEQ ID NO 149)
H[Aib]EGTFTSELATILDEQAARDFIAWLIAHKITDkkkkkk([17-carboxy-Heptadecanoyl]-
isoGlu)];
(SEQ ID NO 150)
H[Aib]EGSFTSELATILDEQAARDFIAWLIEHKITDkkkkkk([17-carboxy-Heptadecanoyl]-
isoGlu)];
(SEQ ID NO 151)
H[Aib]EGTFTSELATILD[K([19-Carboxy-nonadecanoyl]-isoGlu)]QAARDFIAWLIQHKITD;
(SEQ ID NO 152)
H[Aib]EGSFTSE[K([19-carboxy-nonadecanoyl]iso-Glu-Peg3-
Peg3)]ATILDEQAARDFIAWLIEHKITD;
(SEQ ID NO 153)
H[Aib]EGSFTSELATILD[K([19-carboxy-nonadecanoyl]iso-Glu-Peg3-
Peg3)]KAARDFIAWLIEHKITD;
(SEQ ID NO 154)
H[Aib]EGSFTSELATILEG[K([19-carboxy-nonadecanoyl]iso-Glu-Peg3-
Peg3)]AARDFIAWLIEHKITD;
(SEQ ID NO 155)
H[Aib]EGSFTSELATILDEQAA[K([19-carboxy-nonadecanoyl]iso-Glu-Peg3-
Peg3)]DFIAWLIEHKITD;
(SEQ ID NO 156)
H[Aib]EGTFTSELATILDEQAA[K([19-carboxy-nonadecanoyl]iso-Glu-Peg3-
Peg3)]DFIAWLIEHKITD;
(SEQ ID NO 157)
H[Aib]EGTFSSELATILD[K([17-Carboxy-heptadecanoyl]-isoGlu-KEK-
Peg3)]QAARDFIAWLIQHKITD;
(SEQ ID NO 158)
H[Aib]EGTFSSELATILD[K([19-Carboxy-nonadecanoyl]-isoGlu-KEK-
Peg3)]QAARDFIAWLIQHKITD;
(SEQ ID NO 159)
H[Aib]EGTFSSELATILD[K([17-Carboxy-heptadecanoyl]-isoGlu-KEK-
Peg3)]QAARDFIAWLIEHKITD;
(SEQ ID NO 160)
H[Aib]EGTFSSELATILD[K([19-Carboxy-nonadecanoyl]-isoGlu-KEK-
Peg3)]QAARDFIAWLIEHKITD;
(SEQ ID NO 161)
H[Aib]EGTFTSELATILD[K([19-Carboxy-nonadecanoyl]-isoGlu-
KEK)]QAARDFIAWLIQHKITD;
(SEQ ID NO 162)
H[Aib]EGTFTSELATILD[K([19-Carboxy-nonadecanoyl]-isoGlu-KEK-
Peg3)]QAARDFIAWLIQHKITD;
(SEQ ID NO 163)
H[Aib]EGSFTSE[K([19-Carboxy-nonadecanoyl]-isoGlu-KEK-
Peg3)]ATILDEQAARDFIAWLIEHKITD;
(SEQ ID NO 164)
H[Aib]EGTFTSE[K([19-Carboxy-nonadecanoyl]-isoGlu-KEK-
Peg3)]ATILDEQAARDFIAWLIEHKITD;
(SEQ ID NO 165)
H[Aib]EGSFTSE[K([19-carboxy-nonadecanoyl]iso-Glu-KEK-Peg3-
Peg3)]ATILDEQAARDFIAWLIEHKITD;
(SEQ ID NO 166)
H[Aib]EGTFTSELATILD[K([19-Carboxy-nonadecanoyl]-isoGlu-KEK-
Peg3)]QAARDFIAWLIEHKITD;
(SEQ ID NO 167)
H[Aib]EGSFTSELATILD[K([19-Carboxy-nonadecanoyl]-isoGlu-KEK-
Peg3)]QAARDFIAWLIEHKITD;
(SEQ ID NO 168)
H[Aib]EGSFTSELATILD[K([19-Carboxy-nonadecanoyl]-isoGlu-KEK-
Peg3)]QAARDFIAWLIAHKITD;
(SEQ ID NO 169)
H[Aib]EGSFTSELATILD[K([19-Carboxy-nonadecanoyl]-isoGlu-KEK-
Peg3)]KAARDFIAWLIEHKITD;
(SEQ ID NO 170)
H[Aib]EGSFTSELATILD[K([19-carboxy-nonadecanoyl]iso-Glu-KEK-Peg3-
Peg3)]QAARDFIAWLIEHKITD;
(SEQ ID NO 171)
H[Aib]EGSFTSELATILEG[K([19-Carboxy-nonadecanoyl]-isoGlu-KEK-
Peg3)]AARDFIAWLIEHKITD;
(SEQ ID NO 172)
H[Aib]EGSFTSELATILDA[K([19-Carboxy-nonadecanoyl]-isoGlu-KEK-
Peg3)]AARDFIAWLIEHKITD;
(SEQ ID NO 173)
H[Aib]EGSFTSELATILDA[K([19-carboxy-nonadecanoyl]iso-Glu-KEK-Peg3-
Peg3)]AARDFIAWLIEHKITD;
(SEQ ID NO 174)
H[Aib]EGSFTSELATILDEQAA[K([19-Carboxy-nonadecanoyl]-isoGlu-KEK-
Peg3)]DFIAWLIEHKITD;
(SEQ ID NO 175)
H[Aib]EGTFTSELATILDEQAA[K([19-Carboxy-nonadecanoyl]-isoGlu-KEK-
Peg3)]DFIAWLIEHKITD;
(SEQ ID NO 176)
H[Aib]EGSFTSELATILDEQAA[K([19-carboxy-nonadecanoyl]iso-Glu-KEK-Peg3-
Peg3)]DFIAWLIEHKITD;
(SEQ ID NO 177)
H[Aib]EGTFTSELATILDEQAA[K([19-carboxy-nonadecanoyl]iso-Glu-KEK-Peg3-
Peg3)]DFIAWLIEHKITD;
or
(SEQ ID NO 178)
H[Aib]EGSFTSELATILDAKAA[K([19-Carboxy-nonadecanoyl]-isoGlu-KEK-
Peg3)]DFIAWLIEHKITD.

The dual agonist may be:

Hy-H[Aib]EGTFSSELATILD[K([17-carboxy-heptadecanoyl]-
isoGlu)]EAARDFIAWLIEHKITD-OH (Compound 1);
Hy-H[Aib]EGSFTSELATILD[K([17-carboxy-heptadecanoyl]-
isoGlu)]EAARDFIAWLIEHKITD-OH (Compound 2);
Hy-H[Aib]EGTFTSELATILD[K([17-carboxy-heptadecanoyl]-
isoGlu)]EAARDFIAWLIEHKITD-OH (Compound 3);
Hy-H[Aib]EGTFSSELATILD[K([17-carboxy-heptadecanoyl]-
isoGlu)]KAARDFIAWLIEHKITD-OH (Compound 4);
Hy-H[Aib]EGSFTSELATILD[K([17-carboxy-heptadecanoyl]-
isoGlu)]KAARDFIAWLIEHKITD-OH (Compound 5);
Hy-H[Aib]EGTFTSELATILD[K([17-carboxy-heptadecanoyl]-
isoGlu)]KAARDFIAWLIEHKITD-OH (Compound 6);
Hy-H[Aib]EGTFSSELATILDG[K([17-carboxy-heptadecanoyl]-
isoGlu)]AARDFIAWLIEHKITD-OH (Compound 7);
Hy-H[Aib]EGSFTSELATILDG[K([17-carboxy-heptadecanoyl]-
isoGlu)]AARDFIAWLIEHKITD-OH (Compound 8);
Hy-H[Aib]EGTFTSELATILDG[K([17-carboxy-heptadecanoyl]-
isoGlu)]AARDFIAWLIEHKITD-OH (Compound 9);
Hy-H[Aib]EGTFSSELATILD[K([17-carboxy-heptadecanoyl]-
isoGlu)]LAARDFIAWLIEHKITD-OH (Compound 10);
Hy-H[Aib]EGSFTSELATILD[K([17-carboxy-heptadecanoyl]-
isoGlu)]LAARDFIAWLIEHKITD-OH (Compound 11);
Hy-H[Aib]EGTFTSELATILD[K([17-carboxy-heptadecanoyl]-
isoGlu)]LAARDFIAWLIEHKITD-OH (Compound 12);
Hy-H[Aib]EGTFSSELATILD[K([17-carboxy-heptadecanoyl]-
isoGlu)]LAARDFIAWLIAHKITD-OH (Compound 13);
Hy-H[Aib]EGSFTSELATILD[K([17-carboxy-heptadecanoyl]-
isoGlu)]LAARDFIAWLIAHKITD-OH (Compound 14);
Hy-H[Aib]EGTFTSELATILD[K([17-carboxy-heptadecanoyl]-
isoGlu)]LAARDFIAWLIAHKITD-OH (Compound 15);
Hy-H[Aib]EGTFTSELATILD[K([17-carboxy-heptadecanoyl]-
isoGlu)]EAARLFIAWLIEHKITD-OH (Compound 16);
Hy-H[Aib]EGTFSSELATILD[K([17-carboxy-heptadecanoyl]-
isoGlu)]QAARDFIAWLIQHKITD-OH (Compound 17);
Hy-H[Aib]EGSFTSELATILD[K([17-carboxy-heptadecanoyl]-
isoGlu)]QAARDFIAWLIQHKITD-OH (Compound 18);
Hy-H[Aib]EGTFTSELATILD[K([17-carboxy-heptadecanoyl]-
isoGlu)]QAARDFIAWLIQHKITD-OH (Compound 19);
Hy-H[Aib]EGTFSSELATILD[K([17-carboxy-heptadecanoyl]-
isoGlu)]QAARDFIAWLIEHKITD-OH (Compound 20);
Hy-H[Aib]EGTFSSELATILD[K([17-carboxy-heptadecanoyl]-
isoGlu)]QAARDFIAWLIAHKITD-OH (Compound 21);
Hy-H[Aib]EGSFTSELATILD[K([17-carboxy-heptadecanoyl]-
isoGlu)]QAARDFIAWLIAHKITD-OH (Compound 22);
Hy-H[Aib]EGTFTSELATILD[K([17-carboxy-heptadecanoyl]-
isoGlu)]QAARDFIAWLIAHKITD-OH (Compound 23);
Hy-H[Aib]EGSFTSELATILD[K([17-carboxy-heptadecanoyl]-
isoGlu)]QAARDFIAWLIEHKITD-OH (Compound 24);
Hy-H[Aib]EGTFTSELATILD[K([17-carboxy-heptadecanoyl]-
isoGlu)]QAARDFIAWLIEHKITD-OH (Compound 25);
Hy-H[Aib]EGSFTSELATILD[K([17-carboxy-heptadecanoyl]-
isoGlu)]QAARDFIAWLIHHKITD-OH (Compound 26);
Hy-H[Aib]EGSFTSELATILD[K([17-carboxy-heptadecanoyl]-
isoGlu)]QAARDFIAWLIYHKITD-OH (Compound 27);
Hy-H[Aib]EGSFTSELATILD[K([17-carboxy-heptadecanoyl]-
isoGlu)]QAARDFIAWLILHKITD-OH (Compound 28);
Hy-H[Aib]EGSFTSELATILD[K([17-carboxy-heptadecanoyl]-
isoGlu)]QAARDFIAWLIKHKITD-OH (Compound 29);
Hy-H[Aib]EGSFTSELATILD[K([17-carboxy-heptadecanoyl]-
isoGlu)]QAARDFIAWLIRHKITD-OH (Compound 30);
Hy-H[Aib]EGSFTSELATILD[K([17-carboxy-heptadecanoyl]-
isoGlu)]QAARDFIAWLISHKITD-OH (Compound 31).
Hy-H[Aib]EGSFTSELATILD[K([Hexadecanoyl]-βAla)]QAARDFIAWLQQHKITD-OH
(Compound 32);
Hy-H[Aib]EGSFTSELATILD[K([17-carboxy-heptadecanoyl]iso-Glu-
Peg3)]QAARDFIAWLYQHKITD-OH (Compound 33);
Hy-H[Aib]EGSFTSELATILD[K([19-carboxy-nonadecanoyl]iso-Glu-Peg3-
Peg3)]QAARDFIAWLKQHKITD-OH (Compound 34);
Hy-H[Aib]EGSFTSELATILD[K([19-carboxy-nonadecanoyl]iso-Lys-Peg3-Peg3-
Peg3)]QAARDFIAWLIQQKITD-OH (Compound 35);
Hy-H[Aib]EGSFTSELATILD[K(Octadecanoyl)]QAARDFIAWLIQYKITD-OH
(Compound 36);
Hy-H[Aib]EGTFSSELSTILE[K(Hexadecanoyl-isoGlu)]QASREFIAWLIAYKITE-OH
(Compound 37);
Hy-H[Aib]EGTFSSELATILDEQAARDFIAWLIAHKITDkkkkkk([17-carboxy-
Heptadecanoyl]-isoGlu)]-[NH2] (Compound 38);
Hy-H[Aib]EGTFTSELATILDEQAARDFIAWLIAHKITDkkkkkk([17-carboxy-
Heptadecanoyl]-isoGlu)]-[NH2] (Compound 39);
Hy-H[Aib]EGSFTSELATILDEQAARDFIAWLIEHKITDkkkkkk([17-carboxy-
Heptadecanoyl]-isoGlu)]-[NH2] (Compound 40);
Hy-H[Aib]EGTFTSELATILD[K([19-Carboxy-nonadecanoyl]-
isoGlu)]QAARDFIAWLIQHKITD-OH (Compound 41);
Hy-H[Aib]EGSFTSE[K([19-carboxy-nonadecanoyl]iso-Glu-Peg3-
Peg3)]ATILDEQAARDFIAWLIEHKITD-OH (Compound 42);
Hy-H[Aib]EGSFTSELATILD[K([19-carboxy-nonadecanoyl]iso-Glu-Peg3-
Peg3)]KAARDFIAWLIEHKITD-OH (Compound 43);
Hy-H[Aib]EGSFTSELATILEG[K([19-carboxy-nonadecanoyl]iso-Glu-Peg3-
Peg3)]AARDFIAWLIEHKITD-OH (Compound 44);
Hy-H[Aib]EGSFTSELATILDEQAA[K([19-carboxy-nonadecanoyl]iso-Glu-Peg3-
Peg3)]DFIAWLIEHKITD-OH (Compound 45);
Hy-H[Aib]EGTFTSELATILDEQAA[K([19-carboxy-nonadecanoyl]iso-Glu-Peg3-
Peg3)]DFIAWLIEHKITD-OH (Compound 46).
Hy-H[Aib]EGTFSSELATILD[K([17-Carboxy-heptadecanoyl]-isoGlu-KEK-
Peg3)]QAARDFIAWLIQHKITD-OH (Compound 47);
Hy-H[Aib]EGTFSSELATILD[K([19-Carboxy-nonadecanoyl]-isoGlu-KEK-
Peg3)]QAARDFIAWLIQHKITD-OH (Compound 48);
Hy-H[Aib]EGTFSSELATILD[K([17-Carboxy-heptadecanoyl]-isoGlu-KEK-
Peg3)]QAARDFIAWLIEHKITD-OH (Compound 49);
Hy-H[Aib]EGTFSSELATILD[K([19-Carboxy-nonadecanoyl]-isoGlu-KEK-
Peg3)]QAARDFIAWLIEHKITD-OH (Compound 50);
Hy-H[Aib]EGTFTSELATILD[K([19-Carboxy-nonadecanoyl]-isoGlu-
KEK)]QAARDFIAWLIQHKITD-OH (Compound 51);
Hy-H[Aib]EGTFTSELATILD[K([19-Carboxy-nonadecanoyl]-isoGlu-KEK-
Peg3)]QAARDFIAWLIQHKITD-OH (Compound 52);
Hy-H[Aib]EGSFTSE[K([19-Carboxy-nonadecanoyl]-isoGlu-KEK-
Peg3)]ATILDEQAARDFIAWLIEHKITD-OH (Compound 53);
Hy-H[Aib]EGTFTSE[K([19-Carboxy-nonadecanoyl]-isoGlu-KEK-
Peg3)]ATILDEQAARDFIAWLIEHKITD-OH (Compound 54);
Hy-H[Aib]EGSFTSE[K([19-carboxy-nonadecanoyl]iso-Glu-KEK-Peg3-
Peg3)]ATILDEQAARDFIAWLIEHKITD-OH (Compound 55);
Hy-H[Aib]EGTFTSELATILD[K([19-Carboxy-nonadecanoyl]-isoGlu-KEK-
Peg3)]QAARDFIAWLIEHKITD-OH (Compound 56);
Hy-H[Aib]EGSFTSELATILD[K([19-Carboxy-nonadecanoyl]-isoGlu-KEK-
Peg3)]QAARDFIAWLIEHKITD-OH (Compound 57);
Hy-H[Aib]EGSFTSELATILD[K([19-Carboxy-nonadecanoyl]-isoGlu-KEK-
Peg3)]QAARDFIAWLIAHKITD-OH (Compound 58);
Hy-H[Aib]EGSFTSELATILD[K([19-Carboxy-nonadecanoyl]-isoGlu-KEK-
Peg3)]KAARDFIAWLIEHKITD-OH (Compound 59);
Hy-H[Aib]EGSFTSELATILD[K([19-carboxy-nonadecanoyl]iso-Glu-KEK-Peg3-
Peg3)]QAARDFIAWLIEHKITD-OH (Compound 60);
Hy-H[Aib]EGSFTSELATILEG[K([19-Carboxy-nonadecanoyl]-isoGlu-KEK-
Peg3)]AARDFIAWLIEHKITD-OH (Compound 61);
Hy-H[Aib]EGSFTSELATILDA[K([19-Carboxy-nonadecanoyl]-isoGlu-KEK-
Peg3)]AARDFIAWLIEHKITD-OH (Compound 62);
Hy-H[Aib]EGSFTSELATILDA[K([19-carboxy-nonadecanoyl]iso-Glu-KEK-Peg3-
Peg3)]AARDFIAWLIEHKITD-OH (Compound 63);
Hy-H[Aib]EGSFTSELATILDEQAA[K([19-Carboxy-nonadecanoyl]-isoGlu-KEK-
Peg3)]DFIAWLIEHKITD-OH (Compound 64);
Hy-H[Aib]EGTFTSELATILDEQAA[K([19-Carboxy-nonadecanoyl]-isoGlu-KEK-
Peg3)]DFIAWLIEHKITD-OH (Compound 65);
Hy-H[Aib]EGSFTSELATILDEQAA[K([19-carboxy-nonadecanoyl]iso-Glu-KEK-Peg3-
Peg3)]DFIAWLIEHKITD-OH (Compound 66);
Hy-H[Aib]EGTFTSELATILDEQAA[K([19-carboxy-nonadecanoyl]iso-Glu-KEK-Peg3-
Peg3)]DFIAWLIEHKITD-OH (Compound 67);
or
Hy-H[Aib]EGSFTSELATILDAKAA[K([19-Carboxy-nonadecanoyl]-isoGlu-KEK-
Peg3)]DFIAWLIEHKITD-OH (Compound 68).

In one aspect the dual agonist is H[Aib]EGSFTSELATILD[Ψ]QAARDFIAWLIQHKITD (SEQ ID NO 34). In one aspect the dual agonist is:

a.
Hy-H[Aib]EGSFTSELATILD[K([17-carboxy-
heptadecanoyl]-isoGlu)]QAARDFIAWLIQHKITD-OH
(CPD1OH);
or
b.
Hy-H[Aib]EGSFTSELATILD[K([17-carboxy-
heptadecanoyl]-isoGlu)]QAARDFIAWLIQHKITD-NH2
(CPD1NH2).

In a preferred embodiment the dual agonist is Hy-H[Aib]EGSFTSELATILD[K([17-carboxy-heptadecanoyl]-isoGlu)]QAARDFIAWLIQHKITD-OH (Compound 18).

In a preferred embodiment the dual agonist is Hy-H[Aib]EGTFTSELATILD[K([17-carboxy-heptadecanoyl]-isoGlu)]QAARDFIAWLIQHKITD-OH (Compound 19).

The dual agonist may be in the form of a pharmaceutically acceptable salt or solvate, such as a pharmaceutically acceptable acid addition salt.

The invention also provides a composition comprising a dual agonist of the invention, or a pharmaceutically acceptable salt or solvate thereof, together with a carrier, excipient or vehicle for use in a method of reducing or inhibiting weight gain, reducing food intake, reducing appetite, promoting weight loss, or treating obesity, morbid obesity, obesity-linked gallbladder disease, or obesity-induced sleep apnea, wherein the method comprises administering the dual agonist to the patient at a dose of about 0.1 mg to about 10.0 mg. The carrier may be a pharmaceutically acceptable carrier.

The composition may be a pharmaceutical composition. The pharmaceutical composition may be formulated as a liquid suitable for administration by injection or infusion. It may be formulated to achieve slow release of the dual agonist.

The invention also provides a dual agonist according to the invention for use in a method of reducing or inhibiting weight gain, reducing gastric emptying or intestinal transit, reducing food intake, reducing appetite, or promoting weight loss wherein the method comprises administering the dual agonist to the patient at a dose of about 0.1 mg to about 10.0 mg.

The invention also provides a dual agonist according to the invention for use in a method of prophylaxis or treatment of obesity, morbid obesity, obesity-linked gallbladder disease, or obesity-induced sleep apnea wherein the method comprises administering the dual agonist to the patient at a dose of about 0.1 mg to about 10.0 mg.

The invention also provides a method of reducing or inhibiting weight gain, reducing gastric emptying or intestinal transit, reducing food intake, reducing appetite, or promoting weight loss in a subject in need thereof, the method comprising administering a dual agonist according to the invention to the subject at a dose of about 0.1 mg to about 10.0 mg.

The invention also provides a method of prophylaxis or treatment of obesity, morbid obesity, obesity-linked gallbladder disease, or obesity-induced sleep apnea, the method comprising administering a dual agonist according to the invention to the subject at a dose of about 0.1 mg to about 10.0 mg.

The invention also provides the use of a dual agonist according to the invention in the preparation of a medicament for reducing or inhibiting weight gain, reducing gastric emptying or intestinal transit, reducing food intake, reducing appetite, or promoting weight loss wherein the medicament is administered to the patient at a dose of about 0.1 mg to about 10.0 mg.

The invention also provides the use of a dual agonist according to the invention in the preparation of a medicament for prophylaxis or treatment of obesity, morbid obesity, obesity-linked gallbladder disease, or obesity-induced sleep apnea wherein the medicament is administered to the patient at a dose of about 0.1 mg to about 10.0 mg.

A further aspect provides a therapeutic kit comprising a dual agonist, or a pharmaceutically acceptable salt or solvate thereof, according to the invention for use in a method of reducing or inhibiting weight gain, reducing food intake, reducing appetite, promoting weight loss, or treating obesity, morbid obesity, obesity-linked gallbladder disease, or obesity-induced sleep apnea, or for reducing or inhibiting weight gain, reducing gastric emptying or intestinal transit, reducing food intake, reducing appetite, or promoting weight loss, wherein the method comprises administering the dual agonist to the patient at a dose of about 0.1 mg to about 10.0 mg.

In one aspect the patient or subject (terms used interchangeably herein) may experience enhanced satiety following administration of the dual agonist.

### FIGURES

FIG. 1: Mean pharmacokinetic profiles of Cpd. 18 following single dose to healthy subjects.

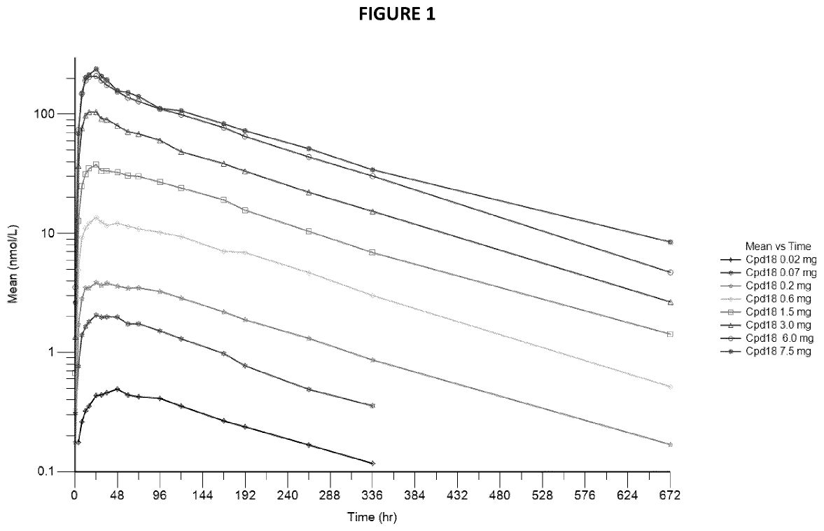

FIG. 2: Multi-ascending dose study design. Upper lines denote number of patients treated with Cpd. 18, lower lines denote placebo administration, diamonds represent safety evaluation.

FIG. 3: Change in body weight following multi-ascending dose in phase 1 b study.

FIG. 4: Illustration of the randomised (2:2:1:1) parallel-group, double-blind, placebo-controlled study design in which 54 individuals with obesity will receive either 1) compound 18 2/4/6 mg, 2) compound 18 2/4 mg, 3) placebo 2/4/6 mg, or Ψ) placebo 2/4 mg for a period of 12 weeks

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

All patents, published patent applications and non-patent publications referred to in this application are specifically incorporated by reference herein. In case of conflict, the present specification, including its specific definitions, will control.

Each embodiment of the invention described herein may be taken alone or in combination with one or more other embodiments of the invention.

### Definitions

Unless specified otherwise, the following definitions are provided for specific terms which are used in the present written description.

Throughout this specification, the word “comprise”, and grammatical variants thereof, such as “comprises” or “comprising”, will be understood to imply the inclusion of a stated integer or component, or group of integers or components, but not the exclusion of any other integer or component, or group of integers or components.

The singular forms “a,” “an,” and “the” include the plurals unless the context clearly dictates otherwise.

The term “including” is used to mean “including but not limited to”. “Including” and “including but not limited to” may be used interchangeably.

The terms “patient”, “subject” and “individual” may be used interchangeably and refer to either a human or a non-human animal. These terms include mammals such as humans, primates, livestock animals (e.g., bovines and porcines), companion animals (e.g., canines and felines) and rodents (e.g., mice and rats).

The term “solvate” in the context of the present invention refers to a complex of defined stoichiometry formed between a solute (in casu, a peptide or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

The term “agonist” as employed in the context of the invention refers to a substance (ligand) that activates the receptor type in question.

Throughout the present description and claims the conventional three-letter and one-letter codes for naturally occurring amino acids are used, i.e.

A (Ala), G (Gly), L (Leu), I (lie), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro);
as well as generally accepted three-letter codes for other α-amino acids, such as sarcosine (Sar), norleucine (Nle), α-aminoisobutyric acid (Aib), 2,3-diaminopropanoic acid (Dap), 2,4-diaminobutanoic acid (Dab) and 2,5-diaminopentanoic acid (ornithine; Orn). Such other α-amino acids may be shown in square brackets “[ ]” (e.g. “[Aib]”) when used in a general formula or sequence in the present specification, especially when the rest of the formula or sequence is shown using the single letter code. Unless otherwise specified, amino acid residues in peptides of the invention are of the L-configuration. However, D-configuration amino acids may be incorporated. In the present context, an amino acid code written with a small letter represents the D-configuration of said amino acid, e.g. “k” represents the D-configuration of lysine (K).

Among sequences disclosed herein are sequences incorporating a “Hy—” moiety at the amino terminus (N-terminus) of the sequence, and either an “—OH” moiety or an “—NH₂” moiety at the carboxy terminus (C-terminus) of the sequence. In such cases, and unless otherwise indicated, a “Hy—” moiety at the N-terminus of the sequence in question indicates a hydrogen atom [i.e. R¹=hydrogen=Hy in the general formulas; corresponding to the presence of a free primary or secondary amino group at the N-terminus], while an “—OH” or an “—NH₂” moiety at the C-terminus of the sequence indicates a hydroxy group [e.g. R²═OH in general formulas; corresponding to the presence of a carboxy (COOH) group at the C-terminus] or an amino group [e.g. R²═[NH₂]in the general formulas; corresponding to the presence of an amido (CONH₂) group at the C-terminus], respectively. In each sequence of the invention, a C-terminal “—OH” moiety may be substituted for a C-terminal “—NH₂” moiety, and vice-versa. “Percent (%) amino acid sequence identity” with respect to the GLP-2 polypeptide sequences is defined as the percentage of amino acid residues in a candidate sequence that are identical to the amino acid residues in the wild-type (human) GLP-2 sequence, after aligning the sequences and introducing gaps, if necessary, to achieve the maximum percent sequence identity, and not considering any conservative substitutions as part of the sequence identity. Sequence alignment can be carried out by the skilled person using techniques well known in the art, for example using publicly available software such as BLAST, BLAST2 or Align software. For examples, see Altschul et al., Methods in Enzymology 266: 460-480 (1996) or Pearson et al., Genomics 46: 24-36, 1997.

The percentage sequence identities used herein in the context of the present invention may be determined using these programs with their default settings. More generally, the skilled worker can readily determine appropriate parameters for determining alignment, including any algorithms needed to achieve maximal alignment over the full length of the sequences being compared.

### Dual Agonist Compounds

In accordance with the present invention, the dual agonist has at least one GLP-1 and at least one GLP-2 biological activity. Exemplary GLP-1 physiological activities include reducing rate of intestinal transit, reducing rate of gastric emptying, reducing appetite, food intake or body weight, and improving glucose control and glucose tolerance. Exemplary GLP-2 physiological activities include causing an increase in intestinal mass (e.g. of small intestine or colon), intestinal repair, and improving intestinal barrier function (i.e. reducing permeability of the intestine). These parameters can be assessed in in vivo assays in which the mass and the permeability of the intestine, or a portion thereof, is determined after a test animal has been treated with a dual agonist.

The dual agonists have agonist activity at the GLP-1 and GLP-2 receptors, e.g. the human GLP-1 and GLP-2 receptors. EC₅₀ values for in vitro receptor agonist activity may be used as a numerical measure of agonist potency at a given receptor. An EC₅₀ value is a measure of the concentration (e.g. mol/L) of a compound required to achieve half of that compound's maximal activity in a particular assay. A compound having a numerical EC₅₀ at a particular receptor which is lower than the EC₅₀ of a reference compound in the same assay may be considered to have higher potency at that receptor than the reference compound.

### GLP-1 Activity

In some embodiments, the dual agonist has an EC₅₀ at the GLP-1 receptor (e.g. the human GLP-1 receptor) which is below 2.0 nM, below 1.5 nM, below 1.0 nM, below 0.9 nM, below 0.8 nM, below 0.7 nM, below 0.6 nM, below 0.5 nM, below 0.4 nM, below 0.3 nM, below 0.2 nM, below 0.1 nM, below 0.09 nM, below 0.08 nM, below 0.07 nM, below 0.06 nM, below 0.05 nM, below 0.04 nM, e.g. when assessed using the GLP-1 receptor potency assay described in the Examples below.

In some embodiments, the dual agonist has an EC₅₀ at the GLP-1 receptor which is between 0.005 and 2.5 nM, between 0.01 nM and 2.5 nM, between 0.025 and 2.5 nM, between 0.005 and 2.0 nM, between 0.01 nM and 2.0 nM, between 0.025 and 2.0 nM, between 0.005 and 1.5 nM, between 0.01 nM and 1.5 nM, between 0.025 and 1.5 nM, between 0.005 and 1.0 nM, between 0.01 nM and 1.0 nM, between 0.025 and 1.0 nM, between 0.005 and 0.5 nM, between 0.01 nM and 0.5 nM, between 0.025 and 0.5 nM, between 0.005 and 0.25 nM, between 0.01 nM and 0.25 nM, between 0.025 and 0.25 nM, e.g. when assessed using the GLP-1 receptor potency assay described in the Examples below.

An alternative measure of GLP-1 agonist activity may be derived by comparing the potency of a dual agonist with the potency of a known (or reference) GLP-1 agonist when both are measured in the same assay. Thus the relative potency at the GLP-1 receptor may be defined as: [EC₅₀ (reference agonist)]/[EC₅₀ (dual agonist)].

Thus a value of 1 indicates that the dual agonist and reference agonist have equal potency, a value of >1 indicates that the dual agonist has higher potency (i.e. lower EC₅₀) than the reference agonist, and a value of <1 indicates that the dual agonist has lower potency (i.e. higher EC₅₀) than the reference agonist.

The reference GLP-1 agonist may, for example, be human GLP-1(7-37), liraglutide (NN2211; Victoza), or Exendin-4, but is preferably liraglutide.

Typically the relative potency will be between 0.001 and 100, e.g. between 0.001 and 10, between 0.001 and 5, between 0.001 and 1, between 0.001 and 0.5, between 0.001 and 0.1, between 0.001 and 0.05, or between 0.001 and 0.01; between 0.01 and 10, between 0.01 and 5, between 0.01 and 1, between 0.01 and 0.5, between 0.01 and 0.1, or between 0.01 and 0.05; between 0.05 and 10, between 0.05 and 5, between 0.05 and 1, between 0.05 and 0.5, or between 0.05 and 0.1; between 0.1 and 10, between 0.1 and 5, between 0.1 and 1, or between 0.1 and 0.5; between 0.5 and 10, between 0.5 and 5, or between 0.5 and 1; between 1 and 10, or between 1 and 5; or between 5 and 10.

The dual agonists described in the examples below have slightly lower GLP-1 potency than liraglutide and so may, for example, have a relative potency between 0.01 and 1, between 0.01 and 0.5 or between 0.01 and 0.1.

By contrast, the dual agonists of the invention have higher potency at the GLP-1 receptor (e.g. the human GLP-1 receptor) than wild type human GLP-2 (hGLP-2 (1-33)) or [Gly2]-hGLP-2 (1-33) (i.e. human GLP-2 having glycine at position 2, also known as teduglutide). Thus, the relative potency of the dual agonists at the GLP-1 receptor compared to hGLP-2 (1-33) or teduglutide is greater than 1, typically greater than 5 or greater than 10, and may be up to 100, up to 500, or even higher.

### GLP-2 Activity

In some embodiments, the dual agonist has an EC₅₀ at the GLP-2 receptor (e.g. the human GLP-2 receptor) which is below 2.0 nM, below 1.5 nM, below 1.0 nM, below 0.9 nM, below 0.8 nM, below 0.7 nM, below 0.6 nM, below 0.5 nM, below 0.4 nM, below 0.3 nM, below 0.2 nM, below 0.1 nM, below 0.09 nM, below 0.08 nM, below 0.07 nM, below 0.06 nM, below 0.05 nM, below 0.04 nM, below 0.03 nM, below 0.02 nM, or below 0.01 nM, e.g. when assessed using the GLP-2 receptor potency assay described in the Examples below.

In some embodiments, the dual agonist has an EC₅₀ at the GLP-2 receptor which is between 0.005 and 2.0 nM, between 0.01 nM and 2.0 nM, between 0.025 and 2.0 nM, between 0.005 and 1.5 nM, between 0.01 nM and 1.5 nM, between 0.025 and 1.5 nM, between 0.005 and 1.0 nM, between 0.01 nM and 1.0 nM, between 0.025 and 1.0 nM, between 0.005 and 0.5 nM, between 0.01 nM and 0.5 nM, between 0.025 and 0.5 nM, between 0.005 and 0.25 nM, between 0.01 nM and 0.25 nM, between 0.025 and 0.25 nM, e.g. when assessed using the GLP-2 receptor potency assay described in the Examples below.

An alternative measure of GLP-2 agonist activity may be derived by comparing the potency of a dual agonist with the potency of a known (or reference) GLP-2 agonist when both are measured in the same assay. Thus the relative potency at the GLP-2 receptor may be defined as:

[EC₅₀ (reference agonist)]/[EC₅₀ (dual agonist)].

Thus a value of 1 indicates that the dual agonist and reference agonist have equal potency, a value of >1 indicates that the dual agonist has higher potency (i.e. lower EC₅₀) than the reference agonist, and a value of <1 indicates that the dual agonist has lower potency (i.e. higher EC₅₀) than the reference agonist.

The reference GLP-2 agonist may, for example, be human GLP-2(1-33) or teduglutide ([Gly2]-hGLP-2 (1-33)), but is preferably teduglutide. Typically the relative potency will be between 0.001 and 100, e.g. between 0.001 and 10, between 0.001 and 5, between 0.001 and 1, between 0.001 and 0.5, between 0.001 and 0.1, between 0.001 and 0.05, or between 0.001 and 0.01; between 0.01 and 10, between 0.01 and 5, between 0.01 and 1, between 0.01 and 0.5, between 0.01 and 0.1, or between 0.01 and 0.05; between 0.05 and 10, between 0.05 and 5, between 0.05 and 1, between 0.05 and 0.5, or between 0.05 and 0.1; between 0.1 and 10, between 0.1 and 5, between 0.1 and 1, or between 0.1 and 0.5; between 0.5 and 10, between 0.5 and 5, or between 0.5 and 1; between 1 and 10, or between 1 and 5; or between 5 and 10. The dual agonists described in the examples below have slightly lower GLP-2 potency than teduglutide and so may, for example, have a relative potency between 0.01 and 1, between 0.01 and 0.5, or between 0.01 and 0.1.

By contrast, the dual agonists of the invention have higher potency at the GLP-2 receptor (e.g. the human GLP-2 receptor) than human GLP-1(7-37), liraglutide (NN2211; Victoza), or Exendin-4. Thus, the relative potency of the dual agonists at the GLP-2 receptor compared to human GLP-1(7-37), liraglutide (NN2211; Victoza), or Exendin-4 is greater than 1, typically greater than 5 or greater than 10, and may be up to 100, up to 500, or even higher (if the reference GLP-1 agonist even exerts detectable activity at the GLP-2 receptor).

It will be understood that the absolute potencies of the dual agonists at each receptor are much less important than the balance between the GLP-1 and GLP-2 agonist activities. Thus it is perfectly acceptable for the absolute GLP-1 or GLP-2 potency to be lower than that of known agonists at those receptors, as long as the dual agonist compound exerts acceptable relative levels of potency at both receptors. Any apparent deficiency in absolute potency can be compensated by an increased dose if required.

### Substituents

The dual agonist of the present invention contains a residue Ψwhich comprises a residue of Lys, Arg, Orn, Dap or Dab in which the side chain is conjugated to a substituent Z¹— or Z¹—Z²— wherein Z¹ represents a moiety CH₃—(CH₂)₁₀₋₂₂—(CO)— or HOOC—(CH₂)₁₀₋₂₂—(CO)— and Z² when present represents a spacer.

The spacer Z² is selected from —Z^{S1}—, —Z^{S1}—Z^{S2}—, —Z^{S2}—Z^{S1}, —Z^{S2}—Z^{S3}—, —Z^{S1}Z^{S3}—, —Z^{S2} Z^{S3}—, —Z^{S3} Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, Z^{S3}Z^{S1} Z^{S2}—, —Z^{S3} Z^{S2}Z^{S1}, Z^{S2}Z^{S3}Z^{S2}—
wherein
Z^{S1} is isoGlu, β-Ala, isoLys, or 4-aminobutanoyl;
Z^{S2} is -(Peg3)ₘ- where m is 1, 2, or 3; and
Z^{S3}— is a peptide sequence of 1-6 amino acid units selected from the group consisting of A, L, S, T, Y, Q, D, E, K, k, R, H, F and G.

In some embodiments, Z² is a spacer of the formula —Z^{S1}—, —Z^{S1}—Z^{S2}—, —Z^{S2}—Z^{S1}, or Z^{S2}, where —Z^{S1}— is isoGlu, β-Ala, isoLys, or 4-aminobutanoyl; and —Z^{S2}— is -(Peg3)m- where m is 1, 2, or 3.

Without wishing to be bound by theory, it is believed that the hydrocarbon chain of Z¹ binds albumin in the blood stream, thus shielding the dual agonists of the present invention from enzymatic degradation, which can enhance the half-life of the dual agonists.

The substituent may also modulate the potency of the dual agonists, with respect to the GLP-2 receptor and/or the GLP-1 receptor.

The substituent Z¹— or Z¹—Z²— is conjugated to the functional group at the distal end of the side-chain from the alpha-carbon of the relevant amino acid residue. The normal ability of the amino acid (Lys, Arg, Orn, Dab, Dap) side-chain in question to participate in interactions mediated by that functional group (e.g. intra- and inter-molecular interactions) may therefore be reduced or completely eliminated by the presence of the substituent. Thus, the overall properties of the dual agonist may be relatively insensitive to changes in the actual amino acid conjugated to the substituent. Consequently, it is believed that any of the residues Lys, Arg, Orn, Dab, or Dap may be present at any position where Ψ is permitted. However, in certain embodiments, it may be advantageous that the amino acid to which the substituent is conjugated is Lys or Orn.

The moiety Z¹ may be covalently bonded to the functional group in the amino acid side-chain, or alternatively may be conjugated to the amino acid side-chain functional group via a spacer Z².

The term “conjugated” is used here to describe the covalent attachment of one identifiable chemical moiety to another, and the structural relationship between such moieties. It should not be taken to imply any particular method of synthesis.

The bonds between Z¹, Z^{S1}, Z^{S2}, Z^{S3} and the amino acid side chain to which the substituent is bound (collectively referred to herein as Ψ) are peptidic. In other words, the units may be joined by amide condensation reactions.

Z¹ comprises a hydrocarbon chain having from 10 to 24 carbon (C) atoms, such as from 10 to 22 C atoms, e.g. from 10 to 20 C atoms. Preferably, it has at least 10 or at least 11 C atoms, and preferably it has 20 C atoms or fewer, e.g. 18 C atoms or fewer. For example, the hydrocarbon chain may contain 12, 13, 14, 15, 16, 17, 18, 19 or 20 carbon atoms. For example, it may contain 18 or 20 carbon atoms.

In some embodiments, Z¹ is a group selected from dodecanoyl, tetradecanoyl, hexadecanoyl, octadecanoyl and eicosanoyl, preferably hexadecanoyl, octadecanoyl or eicosanoyl, more preferably octadecanoyl or eicosanoyl.

Alternative Z¹ groups are derived from long-chain saturated α,ω-dicarboxylic acids of formula HOOC—(CH₂)₁₂₋₂₂—COOH, preferably from long-chain saturated α,ω-dicarboxylic acids having an even number of carbon atoms in the aliphatic chain. For example, Z¹ may be:
13-carboxytridecanoyl, i.e. HOOC—(CH₂)₁₂—(CO)—;
15-carboxypentadecanoyl, i.e. HOOC—(CH₂)₁₄—(CO)—;
17-carboxyheptadecanoyl, i.e. HOOC—(CH₂)₁₆—(CO)—;
19-carboxynonadecanoyl, i.e. HOOC—(CH₂)₁₈—(CO)—; or
21-carboxyheneicosanoyl, i.e. HOOC—(CH₂)₂₀—(CO)—.

As mentioned above, Z¹ may be conjugated to the amino acid side-chain by a spacer Z². When present, the spacer is attached to Z¹ and to the amino acid side-chain.

The spacer Z² has the —Z^{S1}—, —Z^{S1}—Z^{S2}—, —Z^{S2}—Z^{S1}, —Z^{S2}—, —Z^{S3}—, —Z^{S1}Z^{S3}—, —Z^{S2} Z^{S3}—, —Z^{S3} Z^{S1}—, —Z^{S3} Z^{S2}, —Z^{S1} Z^{S2} Z^{S3}—, —Z^{S1} Z^{S3} Z^{S2}—, —Z^{S2} Z^{S1} Z^{S3}—, —Z^{S2} Z^{S3} Z^{S1}—, —Z^{S3} Z^{S1} Z^{S2}—, —Z^{S3} Z^{S2} Z^{S1}—, Z^{S2} Z^{S3} Z^{S2}—; where
—Z^{S1}— is isoGlu, β-Ala, isoLys, or 4-aminobutanoyl;
—Z^{S2}— is -(Peg3)m- where m is 1, 2, or 3; and
—Z^{S3}— is a peptide sequence of 1-6 amino acid units independently selected from the group consisting of A (Ala), L (Leu), S (Ser), T (Thr), Y (Tyr), Q (Gln), D (Asp), E (Glu), K (L-Lys), k (D-Lys), R (Arg), H (His), F (Phe) and G (Gly).

The terms “isoGlu” and “isoLys” indicate residues of amino acids which participate in bonds via their side chain carboxyl or amine functional groups. Thus isoGlu participates in bonds via its alpha amino and side chain carboxyl group, while isoLys participates via its carboxyl and side chain amino groups. In the context of the present specification, the terms “γ-Glu” and “isoGlu” are used interchangeably.

The term Peg3 is used to refer to an 8-amino-3,6-dioxaoctanoyl group.

Z^{S3} may, for example, be 3 to 6 amino acids in length, i.e. 3, 4, 5 or 6 amino acids in length.

In some embodiments, the amino acids of Z^{S3} are independently selected from K, k, E, A, T, I and L, e.g. from K, k, E and A, e.g. from K, k and E.

Typically, Z^{S3} includes at least one charged amino acid (K, k, R or E, e.g. K, k or E) and preferably two or more charged amino acids. In some embodiments it includes at least 2 positively charged amino acids (K, k or R, especially K or k), or at least 1 positively charged amino acid (K, k or R, especially K or k) and at least one negatively charged amino acid (E). In some embodiments, all amino acid residues of Z^{S3} are charged. For example, Z^{S3} may be a chain of alternately positively and negatively charged amino acids.

Examples of Z^{S3} moieties include KEK, EKEKEK (SEQ ID NO 7), kkkkkk (SEQ ID NO 179), EkEkEk (SEQ ID NO 8), AKAAEK (SEQ ID NO 9), AKEKEK (SEQ ID NO 10) and ATILEK (SEQ ID NO 11).

Without being bound by theory, it is believed that the incorporation of Z^{S3} into the linker between the fatty acid chain and the peptide backbone may increase the half-life of the dual agonist by enhancing its affinity for serum albumin.

In some embodiments, —Z²— is —Z^{S1}— or —Z^{S1}—Z^{S2}—; in other words, —Z²— is selected from: isoGlu(Peg3)₀₋₃;
β-Ala(Peg3)₀₋₃;
isoLys(Peg3)₀₋₃; and
4-aminobutanoyl(Peg3)₀₋₃.

Thus, certain examples of substituents Z¹— include
[Dodecanoyl], [Tetradecanoyl], [Hexadecanoyl], [Octadecanoyl], [Eicosanoyl],
[13-Carboxy-tridecanoyl], [15-Carboxy-pentadecanoyl], [17-Carboxy-heptadecanoyl], [19-Carboxy-nonadecanoyl], [21-carboxy-heneicosanoyl].

More broadly, —Z²— may be —Z^{S1}—, —Z^{S1}—Z^{S2}—, —Z^{S3}—Z^{S1}—, —Z^{S1}—Z^{S3}—, —Z^{S1}—Z^{S3}—Z^{S2}—, —Z^{S3}—Z^{S2}—Z^{S1}— or Z^{S3}—.

Thus, —Z²— may be selected from the group consisting of:
isoGlu(Peg3)₀₋₃;
β-Ala(Peg3)₀₋₃;
isoLys(Peg3)₀₋₃;
4-aminobutanoyl(Peg3)₀₋₃;
isoGlu(KEK)(Peg3)₀₋₃;
β-Ala(KEK)(Peg3)₀₋₃;
isoLys(KEK)(Peg3)₀₋₃;
4-aminobutanoyl(KEK)(Peg3)₀₋₃;

(SEQ ID NO 180)
KEK(isoGlu);
(SEQ ID NO 181)
KEK(β-Ala);
(SEQ ID NO 182)
KEK(isoLys);
(SEQ ID NO 183)
KEK(4-aminobutanoyl);
(SEQ ID NO 6)
isoGlu(KEK);
(SEQ ID NO 184)
β-Ala(KEK);
(SEQ ID NO 185)
isoLys(KEK);
(SEQ ID NO 186)
4-aminobutanoyl(KEK);

KEK(isoGlu)(Peg3)₀₋₃;
KEK(β-Ala)(Prg3)₀₋₃;
KEK(isoLys)(Peg3)₀₋₃; and
KEK(4-aminobutanoyl)(Peg3)₀₋₃;

Certain examples of substituents Z¹—Z²— include:
[Dodecanoyl]-isoGlu, [Tetradecanoyl]-isoGlu, [Hexadecanoyl]-isoGlu, [Octadecanoyl]-isoGlu,
[Eicosanoyl]-isoGlu,
[Hexadecanoyl]-βAla, [Octadecanoyl]-βAla, [Eicosanoyl]-βAla, [Tetradecanoyl]-βAla, [Dodecanoyl]-βAla,
[Dodecanoyl]-isoGlu-Peg3, [Tetradecanoyl]-isoGlu-Peg3, [Hexadecanoyl]-isoGlu-Peg3, [Octadecanoyl]-isoGlu-Peg3, [Eicosanoyl]-isoGlu-Peg3,
[Dodecanoyl]- βAla-Peg3, [Tetradecanoyl]- βAla-Peg3, [Hexadecanoyl]- βAla-Peg3, [Octadecanoyl]- βAla-Peg3, [Eicosanoyl]- βAla-Peg3,
[Dodecanoyl]-isoGlu-Peg3-Peg3, [Tetradecanoyl]-isoGlu-Peg3-Peg3, [Hexadecanoyl]-isoGlu-Peg3-Peg3, [Octadecanoyl]-isoGlu-Peg3-Peg3, [Eicosanoyl]-isoGlu-Peg3-Peg3,
[Dodecanoyl]- βAla-Peg3-Peg3, [Tetradecanoyl]- βAla-Peg3-Peg3, [Hexadecanoyl]- βAla-Peg3-Peg3, [Octadecanoyl]- βAla-Peg3-Peg3, [Eicosanoyl]- βAla-Peg3-Peg3,
[Dodecanoyl]-isoGlu-Peg3-Peg3-Peg3, [Tetradecanoyl]-isoGlu-Peg3-Peg3-Peg3,
[Hexadecanoyl]-isoGlu-Peg3-Peg3-Peg3, [Octadecanoyl]-isoGlu-Peg3-Peg3-Peg3,
[Eicosanoyl]-isoGlu-Peg3-Peg3-Peg3,
[Dodecanoyl]- βAla-Peg3-Peg3-Peg3, [Tetradecanoyl]- βAla-Peg3-Peg3-Peg3, [Hexadecanoyl]βAla-Peg3-Peg3-Peg3, [Octadecanoyl]- βAla-Peg3-Peg3-Peg3, [Eicosanoyl]- βAla-Peg3-Peg3-Peg3,
[Dodecanoyl]-isoLys, [Tetradecanoyl]-isoLys, [Hexadecanoyl]-isoLys, [Octadecanoyl]-isoLys,
[Eicosanoyl]-isoLys,
[Hexadecanoyl]-[4-aminobutanoyl], [Octadecanoyl]-[4-aminobutanoyl], [Eicosanoyl]-[4-aminobutanoyl], [Tetradecanoyl]-[4-aminobutanoyl], [Dodecanoyl]-[4-aminobutanoyl],
[Dodecanoyl]-isoLys-Peg3, [Tetradecanoyl]-isoLys-Peg3, [Hexadecanoyl]-isoLys-Peg3,
[Octadecanoyl]-isoLys-Peg3, [Eicosanoyl]-isoLys-Peg3,
[Dodecanoyl]-[4-aminobutanoyl]-Peg3, [Tetradecanoyl]- [4-aminobutanoyl]-Peg3,
[Hexadecanoyl]-[4-aminobutanoyl]-Peg3,
[Octadecanoyl]-[4-aminobutanoyl]-Peg3, [Eicosanoyl]-[4-aminobutanoyl]-Peg3,
[Dodecanoyl]-isoLys-Peg3-Peg3, [Tetradecanoyl]-isoLys-Peg3-Peg3, [Hexadecanoyl]-isoLys-Peg3-Peg3, [Octadecanoyl]-isoLys-Peg3-Peg3, [Eicosanoyl]-isoLys-Peg3-Peg3,
[Dodecanoyl]-[4-aminobutanoyl]-Peg3-Peg3, [Tetradecanoyl]-[4-aminobutanoyl]-Peg3-Peg3,
[Hexadecanoyl]-[4-aminobutanoyl]-Peg3-Peg3, [Octadecanoyl]-[4-aminobutanoyl]-Peg3-Peg3,
[Eicosanoyl]-[4-aminobutanoyl]-Peg3-Peg3,
[Dodecanoyl]-isoLys-Peg3-Peg3-Peg3, [Tetradecanoyl]-isoLys-Peg3-Peg3-Peg3,
[Hexadecanoyl]-isoLys-Peg3-Peg3-Peg3, [Octadecanoyl]-isoLys-Peg3-Peg3-Peg3,
[Eicosanoyl]-isoLys-Peg3-Peg3-Peg3,
[Dodecanoyl]-[4-aminobutanoyl]-Peg3-Peg3-Peg3, [Tetradecanoyl]-[4-aminobutanoyl]-Peg3-Peg3-Peg3, [Hexadecanoyl]-[4-aminobutanoyl]-Peg3-Peg3-Peg3, [Octadecanoyl]-[4— aminobutanoyl]-Peg3-Peg3-Peg3, [Eicosanoyl]-[4-aminobutanoyl]-Peg3-Peg3-Peg3,
[13-carboxy-tridecanoyl]-isoGlu, [15-carboxy-Pentadecanoyl]-isoGlu, [17-carboxy-Heptadecanoyl]-isoGlu, [19-carboxy-Nonadecanoyl]-isoGlu, [21-carboxy-heneicosanoyl]-isoGlu,
[17-carboxy-Heptadecanoyl]-βAla, [19-carboxy-Nonadecanoyl]-βAla, [21-carboxy-heneicosanoyl]-βAla, [15-carboxy-Pentadecanoyl]-βAla, [13-carboxy-tridecanoyl]-βAla,
[13-carboxy-tridecanoyl]-isoGlu-Peg3, [15-carboxy-Pentadecanoyl]-isoGlu-Peg3, [17-carboxy-Heptadecanoyl]-isoGlu-Peg3, [19-carboxy-Nonadecanoyl]-isoGlu-Peg3, [21-carboxy-heneicosanoyl]-isoGlu-Peg3,
[13-carboxy-tridecanoyl]- βAla-Peg3, [15-carboxy-Pentadecanoyl]- βAla-Peg3, [17-carboxy-Heptadecanoyl]- βAla-Peg3, [19-carboxy-Nonadecanoyl]- βAla-Peg3, [21-carboxy-heneicosanoyl]- βAla-Peg3,
[13-carboxy-tridecanoyl]-isoGlu-Peg3-Peg3, [15-carboxy-Pentadecanoyl]-isoGlu-Peg3-Peg3,
[17-carboxy-Heptadecanoyl]-isoGlu-Peg3-Peg3, [19-carboxy-Nonadecanoyl]-isoGlu-Peg3-Peg3, [21-carboxy-heneicosanoyl]-isoGlu-Peg3-Peg3,
[13-carboxy-tridecanoyl]- βAla-Peg3-Peg3, [15-carboxy-Pentadecanoyl]- βAla-Peg3-Peg3, [17— carboxy-Heptadecanoyl]- βAla-Peg3-Peg3, [1 9-carboxy-Nonadecanoyl]- βAla-Peg3-Peg3, [21— carboxy-heneicosanoyl]- βAla-Peg3-Peg3,
[13-carboxy-tridecanoyl]-isoGlu-Peg3-Peg3-Peg3, [15-carboxy-Pentadecanoyl]-isoGlu-Peg3-Peg3-Peg3, [17-carboxy-Heptadecanoyl]-isoGlu-Peg3-Peg3-Peg3, [19-carboxy-Nonadecanoyl]-isoGlu-Peg3-Peg3-Peg3, [21-carboxy-heneicosanoyl]-isoGlu-Peg3-Peg3-Peg3,
[13-carboxy-tridecanoyl]- βAla-Peg3-Peg3-Peg3, [15-carboxy-Pentadecanoyl]- βAla-Peg3-Peg3-Peg3, [17-carboxy-Heptadecanoyl]- βAla-Peg3-Peg3-Peg3, [19-carboxy-Nonadecanoyl]- βAla-Peg3-Peg3-Peg3, [21-carboxy-heneicosanoyl]- βAla-Peg3-Peg3-Peg3,
[13-carboxy-tridecanoyl]-isoLys, [15-carboxy-Pentadecanoyl]-isoLys, [17-carboxy-Heptadecanoyl]-isoLys, [19-carboxy-Nonadecanoyl]-isoLys, [21-carboxy-heneicosanoyl]-isoLys,
[17-carboxy-Heptadecanoyl]-[4-aminobutanoyl], [19-carboxy-Nonadecanoyl]-[4-aminobutanoyl],
[21-carboxy-heneicosanoyl]-[4-aminobutanoyl], [15-carboxy-Pentadecanoyl]-[4-aminobutanoyl],
[13-carboxy-tridecanoyl]-[4-aminobutanoyl],
[13-carboxy-tridecanoyl]-isoLys-Peg3, [1 5-carboxy-Pentadecanoyl]-isoLys-Peg3, [17-carboxy-Heptadecanoyl]-isoLys-Peg3, [1 9-carboxy-Nonadecanoyl]-isoLys-Peg3, [21-carboxy-heneicosanoyl]-isoLys-Peg3,
[13-carboxy-tridecanoyl]-[4-aminobutanoyl]-Peg3, [15-carboxy-Pentadecanoyl]- [4— aminobutanoyl]-Peg3, [17-carboxy-Heptadecanoyl]-[4-aminobutanoyl]-Peg3,
[19-carboxy-Nonadecanoyl]- βAla-Peg3, [21-carboxy-heneicosanoyl]- βAla-Peg3,
[13-carboxy-tridecanoyl]-isoLys-Peg3-Peg3, [1 5-carboxy-Pentadecanoyl]-isoLys-Peg3-Peg3,
[17-carboxy-Heptadecanoyl]-isoLys-Peg3-Peg3, [1 9-carboxy-Nonadecanoyl]-isoLys-Peg3-Peg3, [21-carboxy-heneicosanoyl]-isoLys-Peg3-Peg3,
[13-carboxy-tridecanoyl]-[4-aminobutanoyl]-Peg3-Peg3, [15-carboxy-Pentadecanoyl]-[4— aminobutanoyl]-Peg3-Peg3, [17-carboxy-Heptadecanoyl]-[4-aminobutanoyl]-Peg3-Peg3, [19— carboxy-Nonadecanoyl]-[4-aminobutanoyl]-Peg3-Peg3, [21-carboxy-heneicosanoyl]-[4— aminobutanoyl]-Peg3-Peg3,
[13-carboxy-tridecanoyl]-isoLys-Peg3-Peg3-Peg3, [15-carboxy-Pentadecanoyl]-isoLys-Peg3-Peg3-Peg3, [17-carboxy-Heptadecanoyl]-isoLys-Peg3-Peg3-Peg3, [19-carboxy-Nonadecanoyl]-isoLys-Peg3-Peg3-Peg3, [21-carboxy-heneicosanoyl]-isoLys-Peg3-Peg3-Peg3,
[13-carboxy-tridecanoyl]-[4-aminobutanoyl]-Peg3-Peg3-Peg3, [15-carboxy-Pentadecanoyl]-[4— aminobutanoyl]-Peg3-Peg3-Peg3, [17-carboxy-Heptadecanoyl]-[4-aminobutanoyl]-Peg3-Peg3-Peg3, [19-carboxy-Nonadecanoyl]-[4-aminobutanoyl]-Peg3-Peg3-Peg3 and [21-carboxy-heneicosanoyl]-[4-aminobutanoyl]-Peg3-Peg3-Peg3.

Further examples of substituents Z¹—Z²— include:
[Dodecanoyl]-isoLys, [Tetradecanoyl]-isoLys, [Hexadecanoyl]-isoLys, [Octadecanoyl]-isoLys,
[Eicosanoyl]-isoLys,
[Hexadecanoyl]-[4-aminobutanoyl], [Octadecanoyl]-[4-aminobutanoyl], [Eicosanoyl]-[4— aminobutanoyl], [Tetradecanoyl]-[4-aminobutanoyl], [Dodecanoyl]-[4-aminobutanoyl],
[Hexadecanoyl]-KEK, [Octadecanoyl]- KEK, [Eicosanoyl]- KEK, [Tetradecanoyl]- KEK,
[Dodecanoyl]- KEK,
[Dodecanoyl]-Peg3, [Tetradecanoyl]-Peg3, [Hexadecanoyl]-Peg3,
[Octadecanoyl]-Peg3, [Eicosanoyl]-Peg3,
[Dodecanoyl]-Peg3-Peg3, [Tetradecanoyl]-Peg3-Peg3,
[Hexadecanoyl]-Peg3-Peg3, [Octadecanoyl]-Peg3-Peg3, [Eicosanoyl]-Peg3-Peg3,
[Dodecanoyl]-Peg3-Peg3-Peg3, [Tetradecanoyl]-Peg3-
Peg3-Peg3, [Hexadecanoyl]-Peg3-Peg3-Peg3, [Octadecanoyl]-Peg3-Peg3-Peg3, [Eicosanoyl]-Peg3-Peg3-Peg3,
Dodecanoyl]-isoLys-Peg3, [Tetradecanoyl]-isoLys-Peg3, [Hexadecanoyl]-isoLys-Peg3,
[Octadecanoyl]-isoLys-Peg3, [Eicosanoyl]-isoLys-Peg3,
[Dodecanoyl]-[4-aminobutanoyl]-Peg3, [Tetradecanoyl]- [4-aminobutanoyl]-Peg3,
[Hexadecanoyl]-[4-aminobutanoyl]-Peg3, [Octadecanoyl]-[4-aminobutanoyl]-Peg3, [Eicosanoyl]-[4-aminobutanoyl]-Peg3,
[Dodecanoyl]-KEK-Peg3, [Tetradecanoyl]-KEK-Peg3, [Hexadecanoyl]-KEK-Peg3,
[Octadecanoyl]-KEK-Peg3, [Eicosanoyl]-KEK-Peg3,
[Dodecanoyl]-isoLys-Peg3-Peg3, [Tetradecanoyl]-isoLys-Peg3-Peg3, [Hexadecanoyl]-isoLys-Peg3-Peg3, [Octadecanoyl]-isoLys-Peg3-Peg3, [Eicosanoyl]-isoLys-Peg3-Peg3,
[Dodecanoyl]-[4-aminobutanoyl]-Peg3-Peg3, [Tetradecanoyl]-[4-aminobutanoyl]-Peg3-Peg3,
[Hexadecanoyl]-[4-aminobutanoyl]-Peg3-Peg3, [Octadecanoyl]-[4-aminobutanoyl]-Peg3-Peg3,
[Eicosanoyl]-[4-aminobutanoyl]-Peg3-Peg3,
[Dodecanoyl]-KEK-Peg3-Peg3, [Tetradecanoyl]-KEK-Peg3-Peg3, [Hexadecanoyl]- KEK-Peg3-Peg3, [Octadecanoyl]- KEK -Peg3-Peg3, [Eicosanoyl]- KEK -Peg3-Peg3,
[Dodecanoyl]-isoLys-Peg3-Peg3-Peg3, [Tetradecanoyl]-isoLys-Peg3-Peg3-Peg3,
[Hexadecanoyl]-isoLys-Peg3-Peg3-Peg3, [Octadecanoyl]-isoLys-Peg3-Peg3-Peg3,
[Eicosanoyl]-isoLys-Peg3-Peg3-Peg3,
[Dodecanoyl]-[4-aminobutanoyl]-Peg3-Peg3-Peg3, [Tetradecanoyl]-[4-aminobutanoyl]-Peg3-Peg3-Peg3, [Hexadecanoyl]-[4-aminobutanoyl]-Peg3-Peg3-Peg3, [Octadecanoyl]-[4— aminobutanoyl]-Peg3-Peg3-Peg3, [Eicosanoyl]-[4-aminobutanoyl]-Peg3-Peg3-Peg3, [Dodecanoyl]-KEK-Peg3-Peg3-Peg3, [Tetradecanoyl]-KEK-Peg3-Peg3-Peg3, [Hexadecanoyl]-KEK-Peg3-Peg3-Peg3, [Octadecanoyl]-KEK-Peg3-Peg3-Peg3, [Eicosanoyl]-KEK-Peg3-Peg3-Peg3, [Dodecanoyl]-isoGlu-KEK-Peg3, [Tetradecanoyl]-isoGlu-KEK-Peg3, [Hexadecanoyl]-isoGlu-KEK-Peg3, [Octadecanoyl]-isoGlu-KEK-Peg3, [Eicosanoyl]-isoGlu-KEK-Peg3, [Dodecanoyl]-[4-aminobutanoyl]-KEK-Peg3, [Tetradecanoyl]-[4-aminobutanoyl]-KEK-Peg3, [Hexadecanoyl]-[4-aminobutanoyl]-KEK-Peg3, [Octadecanoyl]-[4-aminobutanoyl]-KEK-Peg3, [Eicosanoyl]-[4-aminobutanoyl]-KEK-Peg3, [Dodecanoyl]-isoLys-KEK-Peg3, [Tetradecanoyl]-isoLys-KEK-Peg3, [Hexadecanoyl]-isoLys-KEK-Peg3, [Octadecanoyl]-isoLys-KEK-Peg3, [Eicosanoyl]-isoLys-KEK-Peg3, [Dodecanoyl]-βAla-KEK-Peg3, [Tetradecanoyl]-βAla-KEK-Peg3, [Hexadecanoyl]-βAla-KEK-Peg3, [Octadecanoyl]-βAla-KEK-Peg3, [Eicosanoyl]-βAla-KEK-Peg3, [Dodecanoyl]-isoGlu-KEK-Peg3-Peg3, [Tetradecanoyl]-isoGlu-KEK-Peg3-Peg3, [Hexadecanoyl]-isoGlu-KEK-Peg3-Peg3, [Octadecanoyl]-isoGlu-KEK-Peg3-Peg3, [Eicosanoyl]-isoGlu-KEK-Peg3-Peg3, [Dodecanoyl]-βAla-KEK-Peg3-Peg3, [Tetradecanoyl]-βAla-KEK-Peg3-Peg3, [Hexadecanoyl]-βAla-KEK-Peg3-Peg3, [Octadecanoyl]-βAla-KEK-Peg3-Peg3, [Eicosanoyl]-βAla-KEK-Peg3-Peg3, [Dodecanoyl]-isoLys-KEK-Peg3-Peg3, [Tetradecanoyl]-isoLys-KEK-Peg3-Peg3, [Hexadecanoyl]-isoLys-KEK-Peg3-Peg3, [Octadecanoyl]-isoLys-KEK-Peg3-Peg3, [Eicosanoyl]-isoLys-KEK-Peg3-Peg3, [Dodecanoyl]-[4-aminobutanoyl]-KEK-Peg3-Peg3, [Tetradecanoyl]-[4-aminobutanoyl]-KEK-Peg3-Peg3, [Hexadecanoyl]-[4-aminobutanoyl]-KEK-Peg3-Peg3, [Octadecanoyl]-[4— aminobutanoyl]-KEK-Peg3-Peg3, [Eicosanoyl]-[4-aminobutanoyl]-KEK-Peg3-Peg3, [Dodecanoyl]-isoGlu-KEK-Peg3-Peg3-Peg3, [Tetradecanoyl]-isoGlu-KEK-Peg3-Peg3-Peg3, [Hexadecanoyl]-isoGlu-KEK-Peg3-Peg3-Peg3, [Octadecanoyl]-isoGlu-KEK-Peg3-Peg3-Peg3, [Eicosanoyl]-isoGlu-KEK-Peg3-Peg3-Peg3, [Dodecanoyl]- βAla-KEK-Peg3-Peg3-Peg3, [Tetradecanoyl]- βAla-KEK-Peg3-Peg3-Peg3, [Hexadecanoyl]- βAla-KEK-Peg3-Peg3-Peg3, [Octadecanoyl]- βAla-KEK-Peg3-Peg3-Peg3, [Eicosanoyl]- βAla-KEK-Peg3-Peg3-Peg3, [Dodecanoyl]-isoLys-KEK-Peg3-Peg3-Peg3, [Tetradecanoyl]-isoLys-KEK-Peg3-Peg3-Peg3, [Hexadecanoyl]-isoLys-KEK-Peg3-Peg3-Peg3, [Octadecanoyl]-isoLys-KEK-Peg3-Peg3-Peg3, [Eicosanoyl]-isoLys-KEK-Peg3-Peg3-Peg3,
[Dodecanoyl]-[4-aminobutanoyl]-KEK-Peg3-Peg3-Peg3, [Tetradecanoyl]-[4-aminobutanoyl]-KEK-Peg3-Peg3-Peg3, [Hexadecanoyl]-[4-aminobutanoyl]-KEK-Peg3-Peg3-Peg3,
[Octadecanoyl]-[4-aminobutanoyl]-KEK-Peg3-Peg3-Peg3, [Eicosanoyl]-[4-aminobutanoyl]-KEK-Peg3-Peg3-Peg3,
[Dodecanoyl]-KEK-isoGlu-Peg3, [Tetradecanoyl]-KEK-isoGlu-Peg3, [Hexadecanoyl]-KEK-isoGlu-Peg3, [Octadecanoyl]-KEK-isoGlu-Peg3, [Eicosanoyl]-KEK-isoGlu-Peg3,
[Dodecanoyl]-KEK-βAla-Peg3, [Tetradecanoyl]-KEK-βAla-Peg3, [Hexadecanoyl]-KEK-βAla-Peg3, [Octadecanoyl]-KEK-βAla-Peg3, [Eicosanoyl]-KEK-βAla-Peg3,
[Dodecanoyl]-KEK-[4-aminobutanoyl]-Peg3, [Tetradecanoyl]-KEK-[4-aminobutanoyl]-Peg3,
[Hexadecanoyl]-KEK-[4-aminobutanoyl]-Peg3, [Octadecanoyl]-KEK-[4-aminobutanoyl]-Peg3,
[Eicosanoyl]-KEK-[4-aminobutanoyl]-Peg3,
[Dodecanoyl]-KEK-isoLys-Peg3, [Tetradecanoyl]-KEK-isoLys-Peg3, [Hexadecanoyl]-KEK-isoLys-Peg3, [Octadecanoyl]-KEK-isoLys-Peg3, [Eicosanoyl]-KEK-isoLys-Peg3,
[Dodecanoyl]-KEK-isoGlu-Peg3-Peg3, [Tetradecanoyl]-KEK-isoGlu-Peg3-Peg3,
[Hexadecanoyl]-KEK-isoGlu-Peg3-Peg3, [Octadecanoyl]-KEK-isoGlu-Peg3-Peg3, [Eicosanoyl]-KEK-isoGlu-Peg3-Peg3,
[Dodecanoyl]-KEK-βAla-Peg3-Peg3, [Tetradecanoyl]-KEK-βAla-Peg3-Peg3, [Hexadecanoyl]-KEK-βAla-Peg3-Peg3, [Octadecanoyl]-KEK-βAla-Peg3-Peg3, [Eicosanoyl]-βAla-KEK-Peg3-Peg3,
[Dodecanoyl]-KEK-[4-aminobutanoyl]-Peg3-Peg3, [Tetradecanoyl]-KEK-[4-aminobutanoyl]-Peg3-Peg3, [Hexadecanoyl]-KEK-[4-aminobutanoyl]-Peg3-Peg3, [Octadecanoyl]-KEK-[4— aminobutanoyl]-Peg3-Peg3, [Eicosanoyl]-KEK-[4-aminobutanoyl]-Peg3-Peg3,
[Dodecanoyl]-KEK-isoLys-Peg3-Peg3, [Tetradecanoyl]-KEK-isoLys-Peg3-Peg3,
[Hexadecanoyl]-KEK-isoLys-Peg3-Peg3, [Octadecanoyl]-KEK-isoLys-Peg3-Peg3, [Eicosanoyl]-KEK-isoLys-Peg3-Peg3,
[Dodecanoyl]-KEK-isoGlu-Peg3-Peg3-Peg3, [Tetradecanoyl]-KEK-isoGlu-Peg3-Peg3-Peg3,
[Hexadecanoyl]-KEK-isoGlu-Peg3-Peg3-Peg3, [Octadecanoyl]-KEK-isoGlu-Peg3-Peg3-Peg3,
[Eicosanoyl]-KEK-isoGlu-Peg3-Peg3-Peg3,
[Dodecanoyl]-KEK-βAla-Peg3-Peg3-Peg3, [Tetradecanoyl]KEK-βAla-Peg3-Peg3-Peg3,
[Hexadecanoyl]- βAla-KEK-Peg3-Peg3-Peg3, [Octadecanoyl]-KEK-βAla-Peg3-Peg3-Peg3,
[Eicosanoyl]-KEK-βAla-Peg3-Peg3-Peg3,
[Dodecanoyl]-KEK-[4-aminobutanoyl]-Peg3-Peg3-Peg3, [Tetradecanoyl]-KEK-[4— aminobutanoyl]-Peg3-Peg3-Peg3, [Hexadecanoyl]-KEK-[4-aminobutanoyl]-Peg3-Peg3-Peg3,
[Octadecanoyl]-KEK-[4-aminobutanoyl]-Peg3-Peg3-Peg3, [Eicosanoyl]-KEK-[4-aminobutanoyl]-Peg3-Peg3-Peg3,
[Dodecanoyl]-KEK-isoLys-Peg3-Peg3-Peg3, [Tetradecanoyl]-KEK-isoLys-Peg3-Peg3-Peg3,
[Hexadecanoyl]-KEK-isoLys-Peg3-Peg3-Peg3, [Octadecanoyl]-KEK-isoLys-Peg3-Peg3-Peg3,
[Eicosanoyl]-KEK-isoLys-Peg3-Peg3-Peg3,
[13-carboxy-tridecanoyl]-isoGlu, [15-carboxy-Pentadecanoyl]-isoGlu, [17-carboxy-Heptadecanoyl]-isoGlu, [19-carboxy-Nonadecanoyl]-isoGlu, [21-carboxy-hen21-carboxy-heneicosanoyl]-isoGlu,
[17-carboxy-Heptadecanoyl]-βAla, [19-carboxy-Nonadecanoyl]-βAla, [21-carboxy-heneicosanoyl]-βAla, [15-carboxy-Pentadecanoyl]-βAla, [13-carboxy-tridecanoyl]-βAla,
[13-carboxy-tridecanoyl]-isoLys, [15-carboxy-Pentadecanoyl]-isoLys, [17-carboxy-Heptadecanoyl]-isoLys, [19-carboxy-Nonadecanoyl]-isoLys, [21-carboxy-heneicosanoyl]-isoLys,
[17-carboxy-Heptadecanoyl]-[4-aminobutanoyl], [19-carboxy-Nonadecanoyl]-[4-aminobutanoyl],
[21-carboxy-heneicosanoyl]-[4-aminobutanoyl], [15-carboxy-Pentadecanoyl]-[4-aminobutanoyl],
[13-carboxy-tridecanoyl]-[4-aminobutanoyl],
[17-carboxy-Heptadecanoyl]-KEK, [19-carboxy-Nonadecanoyl]- KEK, [21-carboxy-heneicosanoyl]- KEK, [15-carboxy-Pentadecanoyl]- KEK, [13-carboxy-tridecanoyl]- KEK,
[13-carboxy-tridecanoyl]-Peg3, [15-carboxy-Pentadecanoyl]-Peg3, [17-carboxy-Heptadecanoyl]-Peg3, [19-carboxy-Nonadecanoyl]-Peg3, [21-carboxy-heneicosanoyl]-Peg3,
[13-carboxy-tridecanoyl]-Peg3-Peg3, [15-carboxy-Pentadecanoyl]-Peg3-Peg3,
[17-carboxy-Heptadecanoyl]-Peg3-Peg3, [19-carboxy-Nonadecanoyl]-Peg3-Peg3, [21-carboxy-heneicosanoyl]-Peg3-Peg3,
[13-carboxy-tridecanoyl]-Peg3-Peg3-Peg3, [15-carboxy-Pentadecanoyl]-Peg3-Peg3-Peg3, [17-carboxy-Heptadecanoyl]-Peg3-Peg3-Peg3, [19-carboxy-Nonadecanoyl]-Peg3-Peg3-Peg3, [21-carboxy-heneicosanoyl]-Peg3-Peg3-Peg3,
[13-carboxy-tridecanoyl]-isoGlu-Peg3, [15-carboxy-Pentadecanoyl]-isoGlu-Peg3, [17-carboxy-Heptadecanoyl]-isoGlu-Peg3, [19-carboxy-Nonadecanoyl]-isoGlu-Peg3, [21-carboxy-heneicosanoyl]-isoGlu-Peg3,
[13-carboxy-tridecanoyl]- βAla-Peg3, [15-carboxy-Pentadecanoyl]- βAla-Peg3, [17-carboxy-Heptadecanoyl]- βAla-Peg3, [19-carboxy-Nonadecanoyl]- βAla-Peg3, [21-carboxy-heneicosanoyl]- βAla-Peg3,
[13-carboxy-tridecanoyl]-isoLys-Peg3, [1 5-carboxy-Pentadecanoyl]-isoLys-Peg3, [17-carboxy-Heptadecanoyl]-isoLys-Peg3, [1 9-carboxy-Nonadecanoyl]-isoLys-Peg3, [21-carboxy-heneicosanoyl]-isoLys-Peg3,
[13-carboxy-tridecanoyl]-[4-aminobutanoyl]-Peg3, [15-carboxy-Pentadecanoyl]- [4— aminobutanoyl]-Peg3, [17-carboxy-Heptadecanoyl]-[4-aminobutanoyl]-Peg3, [19-carboxy-Nonadecanoyl]-[4-aminobutanoyl]-Peg3, [21-carboxy-heneicosanoyl]-[4-aminobutanoyl]-Peg3,
[13-carboxy-tridecanoyl]-KEK-Peg3, [15-carboxy-Pentadecanoyl]-KEK-Peg3, [17-carboxy-Heptadecanoyl]-KEK-Peg3, [19-carboxy-Nonadecanoyl]-KEK-Peg3, [21-carboxy-heneicosanoyl]-KEK-Peg3,
[13-carboxy-tridecanoyl]-isoGlu-Peg3-Peg3, [15-carboxy-Pentadecanoyl]-isoGlu-Peg3-Peg3,
[17-carboxy-Heptadecanoyl]-isoGlu-Peg3-Peg3, [19-carboxy-Nonadecanoyl]-isoGlu-Peg3-Peg3, [21-carboxy-heneicosanoyl]-isoGlu-Peg3-Peg3,
[13-carboxy-tridecanoyl]- βAla-Peg3-Peg3, [15-carboxy-Pentadecanoyl]- βAla-Peg3-Peg3, [17— carboxy-Heptadecanoyl]- βAla-Peg3-Peg3, [1 9-carboxy-Nonadecanoyl]- βAla-Peg3-Peg3, [21— carboxy-heneicosanoyl]- βAla-Peg3-Peg3,
[13-carboxy-tridecanoyl]-isoLys-Peg3-Peg3, [1 5-carboxy-Pentadecanoyl]-isoLys-Peg3-Peg3,
[17-carboxy-Heptadecanoyl]-isoLys-Peg3-Peg3, [1 9-carboxy-Nonadecanoyl]-isoLys-Peg3-Peg3, [21-carboxy-heneicosanoyl]-isoLys-Peg3-Peg3,
[13-carboxy-tridecanoyl]-[4-aminobutanoyl]-Peg3-Peg3, [15-carboxy-Pentadecanoyl]-[4— aminobutanoyl]-Peg3-Peg3, [17-carboxy-Heptadecanoyl]-[4-aminobutanoyl]-Peg3-Peg3, [19— carboxy-Nonadecanoyl]-[4-aminobutanoyl]-Peg3-Peg3, [21-carboxy-heneicosanoyl]-[4— aminobutanoyl]-Peg3-Peg3,
[13-carboxy-tridecanoyl]-KEK-Peg3-Peg3, [15-carboxy-Pentadecanoyl]-KEK-Peg3-Peg3, [17— carboxy-Heptadecanoyl]- KEK-Peg3-Peg3, [19-carboxy-Nonadecanoyl]- KEK -Peg3-Peg3, [21— carboxy-heneicosanoyl]- KEK -Peg3-Peg3,
[13-carboxy-tridecanoyl]-isoGlu-Peg3-Peg3-Peg3, [15-carboxy-Pentadecanoyl]-isoGlu-Peg3-Peg3-Peg3, [17-carboxy-Heptadecanoyl]-isoGlu-Peg3-Peg3-Peg3, [19-carboxy-Nonadecanoyl]-isoGlu-Peg3-Peg3-Peg3, [21-carboxy-heneicosanoyl]-isoGlu-Peg3-Peg3-Peg3,
[13-carboxy-tridecanoyl]- βAla-Peg3-Peg3-Peg3, [15-carboxy-Pentadecanoyl]- βAla-Peg3-Peg3-Peg3, [17-carboxy-Heptadecanoyl]- βAla-Peg3-Peg3-Peg3, [19-carboxy-Nonadecanoyl]- βAla-Peg3-Peg3-Peg3, [21-carboxy-heneicosanoyl]- βAla-Peg3-Peg3-Peg3,
[13-carboxy-tridecanoyl]-isoLys-Peg3-Peg3-Peg3, [15-carboxy-Pentadecanoyl]-isoLys-Peg3-Peg3-Peg3, [17-carboxy-Heptadecanoyl]-isoLys-Peg3-Peg3-Peg3, [19-carboxy-Nonadecanoyl]-isoLys-Peg3-Peg3-Peg3, [21-carboxy-heneicosanoyl]-isoLys-Peg3-Peg3-Peg3,
[13-carboxy-tridecanoyl]-[4-aminobutanoyl]-Peg3-Peg3-Peg3, [15-carboxy-Pentadecanoyl]-[4— aminobutanoyl]-Peg3-Peg3-Peg3, [17-carboxy-Heptadecanoyl]-[4-aminobutanoyl]-Peg3-Peg3-Peg3, [19-carboxy-Nonadecanoyl]-[4-aminobutanoyl]-Peg3-Peg3-Peg3, [21-carboxy-heneicosanoyl]-[4-aminobutanoyl]-Peg3-Peg3-Peg3,
[13-carboxy-tridecanoyl]- KEK-Peg3-Peg3-Peg3, [15-carboxy-Pentadecanoyl]-KEK-Peg3-Peg3-Peg3, [17-carboxy-Heptadecanoyl]-KEK-Peg3-Peg3-Peg3, [19-carboxy-Nonadecanoyl]-KEK-Peg3-Peg3-Peg3, [21-carboxy-heneicosanoyl]-KEK-Peg3-Peg3-Peg3,
[13-carboxy-tridecanoyl]-isoGlu-KEK-Peg3, [15-carboxy-Pentadecanoyl]-isoGlu-KEK-Peg3, [17— carboxy-Heptadecanoyl]-isoGlu-KEK-Peg3, [19-carboxy-Nonadecanoyl]-isoGlu-KEK-Peg3, [21— carboxy-heneicosanoyl]-isoGlu-KEK-Peg3,
[13-carboxy-tridecanoyl]-[4-aminobutanoyl]-KEK-Peg3, [15-carboxy-Pentadecanoyl]-[4— aminobutanoyl]-KEK-Peg3, [17-carboxy-Heptadecanoyl]-[4-aminobutanoyl]-KEK-Peg3, [19— carboxy-Nonadecanoyl]-[4-aminobutanoyl]-KEK-Peg3, [21-carboxy-heneicosanoyl]-[4— aminobutanoyl]-KEK-Peg3,
[13-carboxy-tridecanoyl]-isoLys-KEK-Peg3, [1 5-carboxy-Pentadecanoyl]-isoLys-KEK-Peg3, [17— carboxy-Heptadecanoyl]-isoLys-KEK-Peg3, [19-carboxy-Nonadecanoyl]-isoLys-KEK-Peg3, [21— carboxy-heneicosanoyl]-isoLys-KEK-Peg3,
[13-carboxy-tridecanoyl]-βAla-KEK-Peg3, [15-carboxy-Pentadecanoyl]-βAla-KEK-Peg3, [17— carboxy-Heptadecanoyl]-βAla-KEK-Peg3, [19-carboxy-Nonadecanoyl]-βAla-KEK-Peg3, [21— carboxy-heneicosanoyl]-βAla-KEK-Peg3,
[13-carboxy-tridecanoyl]-isoGlu-KEK-Peg3-Peg3, [15-carboxy-Pentadecanoyl]-isoGlu-KEK-Peg3-Peg3, [17-carboxy-Heptadecanoyl]-isoGlu-KEK-Peg3-Peg3, [19-carboxy-Nonadecanoyl]-isoGlu-KEK-Peg3-Peg3, [21-carboxy-heneicosanoyl]-isoGlu-KEK-Peg3-Peg3,
[13-carboxy-tridecanoyl]-βAla-KEK-Peg3-Peg3, [15-carboxy-Pentadecanoyl]-βAla-KEK-Peg3-Peg3, [17-carboxy-Heptadecanoyl]-βAla-KEK-Peg3-Peg3, [19-carboxy-Nonadecanoyl]-βAla-KEK-Peg3-Peg3, [21-carboxy-heneicosanoyl]-βAla-KEK-Peg3-Peg3,
[13-carboxy-tridecanoyl]-isoLys-KEK-Peg3-Peg3, [1 5-carboxy-Pentadecanoyl]-isoLys-KEK-Peg3-Peg3, [17-carboxy-Heptadecanoyl]-isoLys-KEK-Peg3-Peg3, [19-carboxy-Nonadecanoyl]-isoLys-KEK-Peg3-Peg3, [21-carboxy-heneicosanoyl]-isoLys-KEK-Peg3-Peg3,
[13-carboxy-tridecanoyl]-[4-aminobutanoyl]-KEK-Peg3-Peg3, [15-carboxy-Pentadecanoyl]-[4— aminobutanoyl]-KEK-Peg3-Peg3, [17-carboxy-Heptadecanoyl]-[4-aminobutanoyl]-KEK-Peg3-Peg3, [19-carboxy-Nonadecanoyl]-[4-aminobutanoyl]-KEK-Peg3-Peg3, [21-carboxy-heneicosanoyl]-[4-aminobutanoyl]-KEK-Peg3-Peg3,
[13-carboxy-tridecanoyl]-isoGlu-KEK-Peg3-Peg3-Peg3, [15-carboxy-Pentadecanoyl]-isoGlu-KEK-Peg3-Peg3-Peg3, [17-carboxy-Heptadecanoyl]-isoGlu-KEK-Peg3-Peg3-Peg3, [19— carboxy-Nonadecanoyl]-isoGlu-KEK-Peg3-Peg3-Peg3, [21-carboxy-heneicosanoyl]-isoGlu-KEK-Peg3-Peg3-Peg3,
[13-carboxy-tridecanoyl]- βAla-KEK-Peg3-Peg3-Peg3, [15-carboxy-Pentadecanoyl]- βAla-KEK-Peg3-Peg3-Peg3, [17-carboxy-Heptadecanoyl]- βAla-KEK-Peg3-Peg3-Peg3, [19-carboxy-Nonadecanoyl]- βAla-KEK-Peg3-Peg3-Peg3, [21-carboxy-heneicosanoyl]- βAla-KEK-Peg3-Peg3-Peg3,
[13-carboxy-tridecanoyl]-isoLys-KEK-Peg3-Peg3-Peg3, [15-carboxy-Pentadecanoyl]-isoLys-KEK-Peg3-Peg3-Peg3, [17-carboxy-Heptadecanoyl]-isoLys-KEK-Peg3-Peg3-Peg3, [19— carboxy-Nonadecanoyl]-isoLys-KEK-Peg3-Peg3-Peg3, [21-carboxy-heneicosanoyl]-isoLys-KEK-Peg3-Peg3-Peg3,
[13-carboxy-tridecanoyl]-[4-aminobutanoyl]-KEK-Peg3-Peg3-Peg3, [15-carboxy-Pentadecanoyl]-[4-aminobutanoyl]-KEK-Peg3-Peg3-Peg3, [17-carboxy-Heptadecanoyl]-[4— aminobutanoyl]-KEK-Peg3-Peg3-Peg3, [19-carboxy-Nonadecanoyl]-[4-aminobutanoyl]-KEK-Peg3-Peg3-Peg3, [21-carboxy-heneicosanoyl]-[4-aminobutanoyl]-KEK-Peg3-Peg3-Peg3,
[13-carboxy-tridecanoyl]-KEK-isoGlu-Peg3, [15-carboxy-Pentadecanoyl]-KEK-isoGlu-Peg3, [17— carboxy-Heptadecanoyl]-KEK-isoGlu-Peg3, [19-carboxy-Nonadecanoyl]-KEK-isoGlu-Peg3, [21— carboxy-heneicosanoyl]-KEK-isoGlu-Peg3,
[13-carboxy-tridecanoyl]-KEK-βAla-Peg3, [15-carboxy-Pentadecanoyl]-KEK-βAla-Peg3, [17— carboxy-Heptadecanoyl]-KEK-βAla-Peg3, [19-carboxy-Nonadecanoyl]-KEK-βAla-Peg3, [21— carboxy-heneicosanoyl]-KEK-βAla-Peg3,
[13-carboxy-tridecanoyl]-KEK-[4-aminobutanoyl]-Peg3, [15-carboxy-Pentadecanoyl]-KEK-[4— aminobutanoyl]-Peg3, [17-carboxy-Heptadecanoyl]-KEK-[4-aminobutanoyl]-Peg3, [19-carboxy-Nonadecanoyl]-KEK-[4-aminobutanoyl]-Peg3, [21-carboxy-heneicosanoyl]-KEK-[4— aminobutanoyl]-Peg3,
[13-carboxy-tridecanoyl]-KEK-isoLys-Peg3, [1 5-carboxy-Pentadecanoyl]-KEK-isoLys-Peg3, [17— carboxy-Heptadecanoyl]-KEK-isoLys-Peg3, [19-carboxy-Nonadecanoyl]-KEK-isoLys-Peg3, [21— carboxy-heneicosanoyl]-KEK-isoLys-Peg3,
[13-carboxy-tridecanoyl]-KEK-isoGlu-Peg3-Peg3, [15-carboxy-Pentadecanoyl]-KEK-isoGlu-Peg3-Peg3, [17-carboxy-Heptadecanoyl]-KEK-isoGlu-Peg3-Peg3, [19-carboxy-Nonadecanoyl]-KEK-isoGlu-Peg3-Peg3, [21-carboxy-heneicosanoyl]-KEK-isoGlu-Peg3-Peg3,
[13-carboxy-tridecanoyl]-KEK-βAla-Peg3-Peg3, [15-carboxy-Pentadecanoyl]-KEK-βAla-Peg3-Peg3, [17-carboxy-Heptadecanoyl]-KEK-βAla-Peg3-Peg3, [19-carboxy-Nonadecanoyl]-KEK-βAla-Peg3-Peg3, [21-carboxy-heneicosanoyl]-βAla-KEK-Peg3-Peg3,
[13-carboxy-tridecanoyl]-KEK-[4-aminobutanoyl]-Peg3-Peg3, [15-carboxy-Pentadecanoyl]-KEK-[4-aminobutanoyl]-Peg3-Peg3, [17-carboxy-Heptadecanoyl]-KEK-[4-aminobutanoyl]-Peg3-Peg3, [19-carboxy-Nonadecanoyl]-KEK-[4-aminobutanoyl]-Peg3-Peg3, [21-carboxy-heneicosanoyl]-KEK-[4-aminobutanoyl]-Peg3-Peg3,
[13-carboxy-tridecanoyl]-KEK-isoLys-Peg3-Peg3, [1 5-carboxy-Pentadecanoyl]-KEK-isoLys-Peg3-Peg3, [17-carboxy-Heptadecanoyl]-KEK-isoLys-Peg3-Peg3, [19-carboxy-Nonadecanoyl]-KEK-isoLys-Peg3-Peg3, [21-carboxy-heneicosanoyl]-KEK-isoLys-Peg3-Peg3,
[13-carboxy-tridecanoyl]-KEK-isoGlu-Peg3-Peg3-Peg3, [15-carboxy-Pentadecanoyl]-KEK-isoGlu-Peg3-Peg3-Peg3, [17-carboxy-Heptadecanoyl]-KEK-isoGlu-Peg3-Peg3-Peg3, [19— carboxy-Nonadecanoyl]-KEK-isoGlu-Peg3-Peg3-Peg3, [21-carboxy-heneicosanoyl]-KEK-isoGlu-Peg3-Peg3-Peg3,
[13-carboxy-tridecanoyl]-KEK-βAla-Peg3-Peg3-Peg3, [15-carboxy-Pentadecanoyl]KEK-βAla-Peg3-Peg3-Peg3, [17-carboxy-Heptadecanoyl]- βAla-KEK-Peg3-Peg3-Peg3, [19-carboxy-Nonadecanoyl]-KEK-βAla-Peg3-Peg3-Peg3, [21-carboxy-heneicosanoyl]-KEK-βAla-Peg3-Peg3-Peg3,
[13-carboxy-tridecanoyl]-KEK-[4-aminobutanoyl]-Peg3-Peg3-Peg3, [15-carboxy-Pentadecanoyl]-KEK-[4-aminobutanoyl]-Peg3-Peg3-Peg3, [17-carboxy-Heptadecanoyl]-KEK-[4— aminobutanoyl]-Peg3-Peg3-Peg3, [19-carboxy-Nonadecanoyl]-KEK-[4-aminobutanoyl]-Peg3-Peg3-Peg3, [21-carboxy-heneicosanoyl]-KEK-[4-aminobutanoyl]-Peg3-Peg3-Peg3,
[13-carboxy-tridecanoyl]-KEK-isoLys-Peg3-Peg3-Peg3, [15-carboxy-Pentadecanoyl]-KEK-isoLys-Peg3-Peg3-Peg3, [17-carboxy-Heptadecanoyl]-KEK-isoLys-Peg3-Peg3-Peg3, [19— carboxy-Nonadecanoyl]-KEK-isoLys-Peg3-Peg3-Peg3, [21-carboxy-heneicosanoyl]-KEK-isoLys-Peg3-Peg3-Peg3.

Certain preferred substituents Z¹— and Z, —Z²— include:
[Hexadecanoyl], [Octadecanoyl], [17-Carboxy-heptadecanoyl], [19-Carboxy-nonadecanoyl],
[Hexadecanoyl]-isoGlu, [Octadecanoyl]-isoGlu,
[Hexadecanoyl]-βAla, [Octadecanoyl]-βAla,
[Hexadecanoyl]-isoGlu-Peg3,
[Hexadecanoyl]-βAla-Peg3,
[Hexadecanoyl]-isoGlu-Peg3-Peg3,
[Hexadecanoyl]-βAla-Peg3-Peg3,
[Hexadecanoyl]-βAla-Peg3-Peg3-Peg3,
[Hexadecanoyl]-isoLys,
[Hexadecanoyl]-[4-aminobutanoyl],
[Hexadecanoyl]-isoLys-Peg3,
[Hexadecanoyl]-[4-aminobutanoyl]-Peg3,
[Hexadecanoyl]-isoLys-Peg3-Peg3,
[Hexadecanoyl]-[4-aminobutanoyl]-Peg3-Peg3,
[Hexadecanoyl]-isoLys-Peg3-Peg3-Peg3,
[Hexadecanoyl]-[4-aminobutanoyl]-Peg3-Peg3-Peg3,
[17-carboxy-Heptadecanoyl]-isoGlu,
[19-carboxy-Nonadecanoyl]-isoGlu,
[17-carboxy-Heptadecanoyl]-βAla,
[19-carboxy-Nonadecanoyl]-βAla,
[17-carboxy-Heptadecanoyl]-isoGlu-Peg3,
[19-carboxy-Nonadecanoyl]-isoGlu-Peg3,
[17-carboxy-Heptadecanoyl]-βAla-Peg3,
[19-carboxy-Nonadecanoyl]-βAla-Peg3,
[17-carboxy-Heptadecanoyl]-isoGlu-Peg3-Peg3,
[19-carboxy-Nonadecanoyl]-isoGlu-Peg3-Peg3,
[17-carboxy-Heptadecanoyl]-βAla-Peg3-Peg3,
[19-carboxy-Nonadecanoyl]-βAla-Peg3-Peg3,
[17-carboxy-Heptadecanoyl]-isoGlu-Peg3-Peg3-Peg3,
[19-carboxy-Nonadecanoyl]-isoGlu-Peg3-Peg3-Peg3,
[17-carboxy-Heptadecanoyl]-βAla-Peg3-Peg3-Peg3,
[19-carboxy-Nonadecanoyl]-βAla-Peg3-Peg3-Peg3,
[17-carboxy-Heptadecanoyl]-isoLys,
[19-carboxy-Nonadecanoyl]-isoLys,
[17-carboxy-Heptadecanoyl]-[4-aminobutanoyl],
[19-carboxy-Nonadecanoyl]-[4-aminobutanoyl],
[17-carboxy-Heptadecanoyl]-isoLys-Peg3,
[19-carboxy-Nonadecanoyl]-isoLys-Peg3,
[17-carboxy-Heptadecanoyl]-[4-aminobutanoyl]-Peg3,
[19-carboxy-Nonadecanoyl]- [4-aminobutanoyl]-Peg3,
[17-carboxy-Heptadecanoyl]-isoLys-Peg3-Peg3,
[19-carboxy-Nonadecanoyl]-isoLys-Peg3-Peg3,
[17-carboxy-Heptadecanoyl]-[4-aminobutanoyl]-Peg3-Peg3,
[19-carboxy-Nonadecanoyl]-[4-aminobutanoyl]-Peg3-Peg3,
[17-carboxy-Heptadecanoyl]-isoLys-Peg3-Peg3-Peg3,
[19-carboxy-Nonadecanoyl]-isoLys-Peg3-Peg3-Peg3,
[17-carboxy-Heptadecanoyl]-[4-aminobutanoyl]-Peg3-Peg3-Peg3,
[19-carboxy-Nonadecanoyl]-[4-aminobutanoyl]-Peg3-Peg3-Peg3.

More preferred substituents Z¹—Z²— include:
[Hexadecanoyl]-isoGlu,
[Hexadecanoyl]-βAla,
[Hexadecanoyl]-isoGlu-Peg3,
[Hexadecanoyl]-βAla-Peg3,
[Hexadecanoyl]-isoGlu-Peg3-Peg3,
[Hexadecanoyl]-isoLys,
[Hexadecanoyl]-isoLys-Peg3,
[Hexadecanoyl]-isoLys-Peg3-Peg3,
[17-carboxy-Heptadecanoyl]-isoGlu,
[19-carboxy-Nonadecanoyl]-isoGlu,
[17-carboxy-Heptadecanoyl]-isoGlu-Peg3,
[19-carboxy-Nonadecanoyl]-isoGlu-Peg3,
[17-carboxy-Heptadecanoyl]-isoGlu-Peg3-Peg3,
[19-carboxy-Nonadecanoyl]-isoGlu-Peg3-Peg3,
[17-carboxy-Heptadecanoyl]-isoGlu-Peg3-Peg3-Peg3,
[19-carboxy-Nonadecanoyl]-isoGlu-Peg3-Peg3-Peg3,
[17-carboxy-Heptadecanoyl]-isoLys,
[19-carboxy-Nonadecanoyl]-isoLys,
[17-carboxy-Heptadecanoyl]-isoLys-Peg3,
[19-carboxy-Nonadecanoyl]-isoLys-Peg3,
[17-carboxy-Heptadecanoyl]-isoLys-Peg3-Peg3,
[19-carboxy-Nonadecanoyl]-isoLys-Peg3-Peg3,
[17-carboxy-Heptadecanoyl]-isoLys-Peg3-Peg3-Peg3,
[19-carboxy-Nonadecanoyl]-isoLys-Peg3-Peg3-Peg3.

Yet further preferred substituents Z¹—Z²— include:
[Hexadecanoyl]-KEK, [Octadecanoyl]-KEK,
[Hexadecanoyl]-βAla-Peg3,
[Hexadecanoyl]-KEK-Peg3,
[Hexadecanoyl]-KEK-Peg3-Peg3,
[Hexadecanoyl]-KEK-Peg3-Peg3-Peg3,
[17-carboxy-Heptadecanoyl]-KEK,
[19-carboxy-Nonadecanoyl]-KEK,
[17-carboxy-Heptadecanoyl]-KEK-Peg3,
[19-carboxy-Nonadecanoyl]-KEK-Peg3,
[17-carboxy-Heptadecanoyl]-KEK-Peg3-Peg3,
[19-carboxy-Nonadecanoyl]-KEK-Peg3-Peg3,
[17-carboxy-Heptadecanoyl]-isoGlu-KEK
[19-carboxy-Nonadecanoyl]-isoGlu-KEK,
[17-carboxy-Heptadecanoyl]-isoLys-KEK
[19-carboxy-Nonadecanoyl]-isoLys-KEK,
[17-carboxy-Heptadecanoyl]-βAla-KEK
[19-carboxy-Nonadecanoyl]-βAla-KEK, [17-carboxy-Heptadecanoyl]-KEK-Peg3-Peg3-Peg3,
[19-carboxy-Nonadecanoyl]-KEK-Peg3-Peg3-Peg3,
[17-carboxy-Heptadecanoyl]-[4-aminobutanoyl]-KEK,
[19-carboxy-Nonadecanoyl]-[4-aminobutanoyl]-KEK,
[17-carboxy-Heptadecanoyl]-[4-aminobutanoyl]-KEK-Peg3-Peg3-Peg3,
[19-carboxy-Nonadecanoyl]-[4-aminobutanoyl]-KEK-Peg3-Peg3-Peg3,
[17-carboxy-Heptadecanoyl]-[4-aminobutanoyl]-KEK-Peg3-Peg3,
[19-carboxy-Nonadecanoyl]-[4-aminobutanoyl]-KEK-Peg3-Peg3,
[Hexadecanoyl]-isoGlu-KEK-Peg3,
[Hexadecanoyl]-isoGlu-KEK-Peg3-Peg3,
[19-carboxy-Nonadecanoyl]-isoGlu-KEK,
[19-carboxy-Nonadecanoyl]-[4-aminobutanoyl]-KEK,
[17-carboxy-Heptadecanoyl]-[4-aminobutanoyl]-KEK-Peg3-Peg3-Peg3,
[17-carboxy-Heptadecanoyl]-[4-aminobutanoyl]-KEK-Peg3-Peg3,
[19-carboxy-Nonadecanoyl]-[4-aminobutanoyl]-KEK-Peg3-Peg3,
[17-carboxy-Heptadecanoyl]-KEK-Peg3-Peg3,
[19-carboxy-Nonadecanoyl]-KEK-Peg3-Peg3,
[17-carboxy-Heptadecanoyl]-isoGlu-KEK-Peg3,
[19-carboxy-Nonadecanoyl]-isoGlu-KEK-Peg3,
[17-carboxy-Heptadecanoyl]-isoGlu-KEK-Peg3-Peg3,
[19-carboxy-Nonadecanoyl]-isoGlu-KEK-Peg3-Peg3.

Examples of Ψ comprising different substituents (fatty acids, FA), conjugated to the amino acid side-chain, optionally by a spacer, are illustrated below:

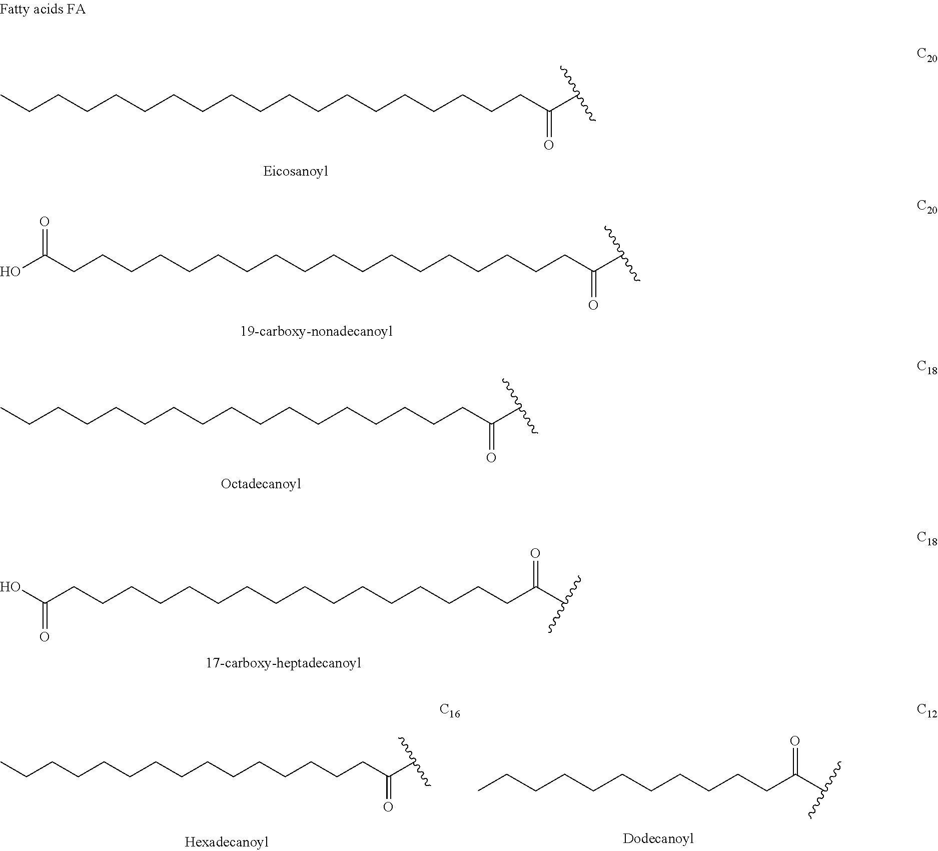 [SMILES: *C(=O)CCCCCCCCCCCCCCCCCCC ; *C(=O)CCCCCCCCCCCCCCCCCCC(=O)O ; *C(=O)CCCCCCCCCCCCCCCCC ; *C(=O)CCCCCCCCCCCCCCCCC(=O)O ; *C(=O)CCCCCCCCCCCCCCC ; *C(=O)CCCCCCCCCCC]

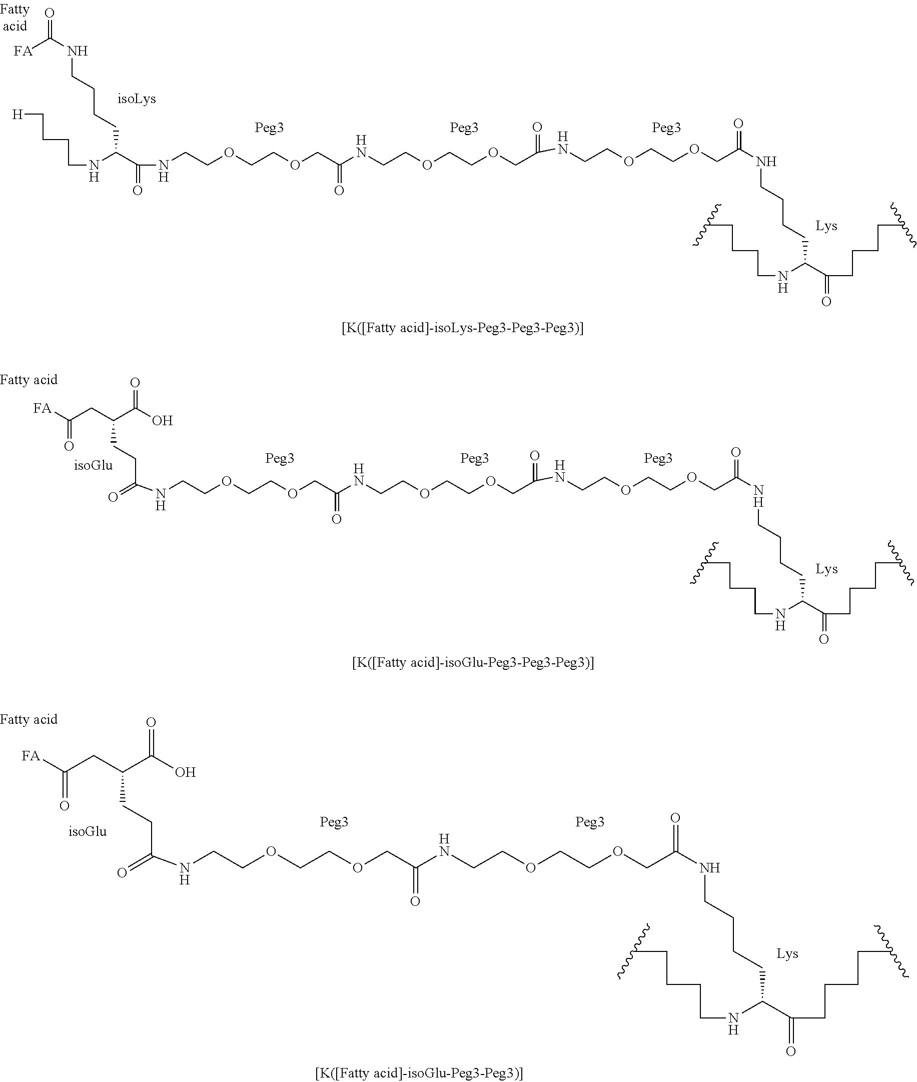

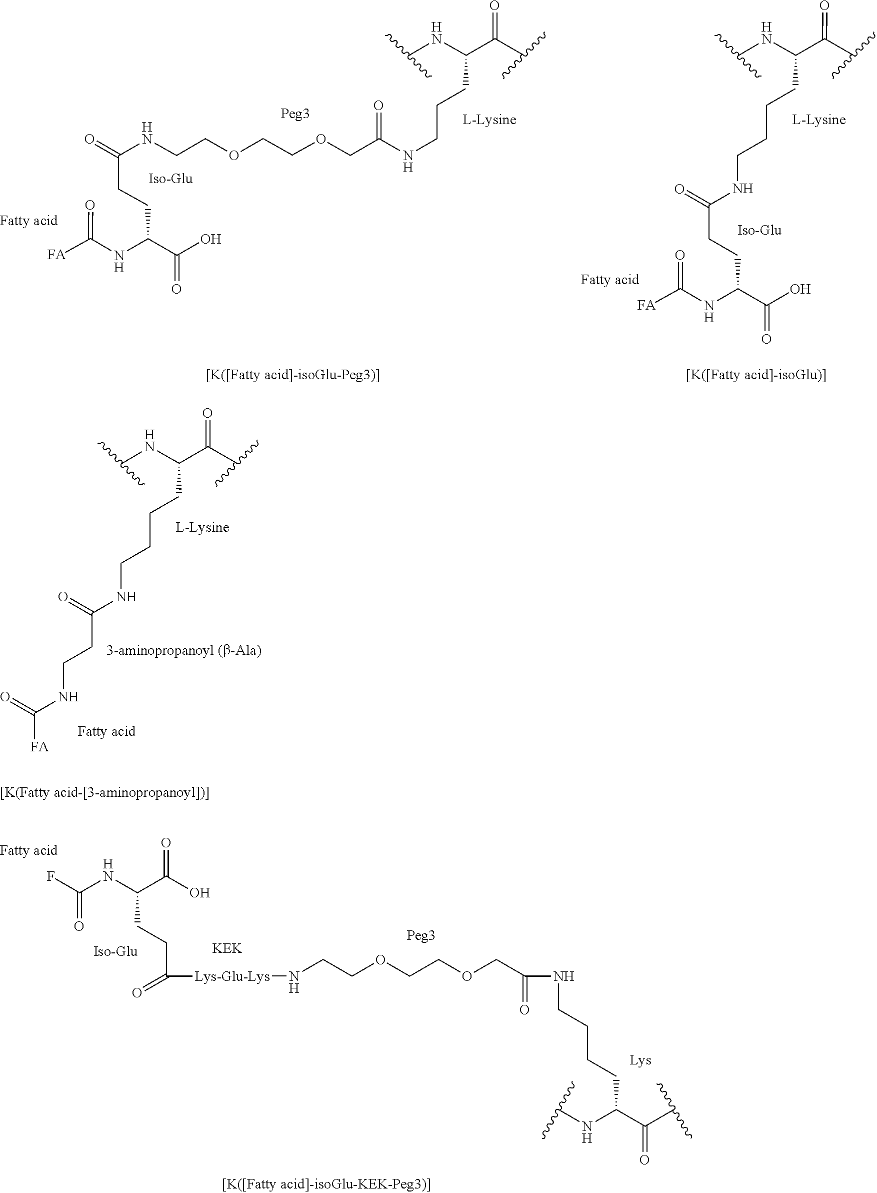

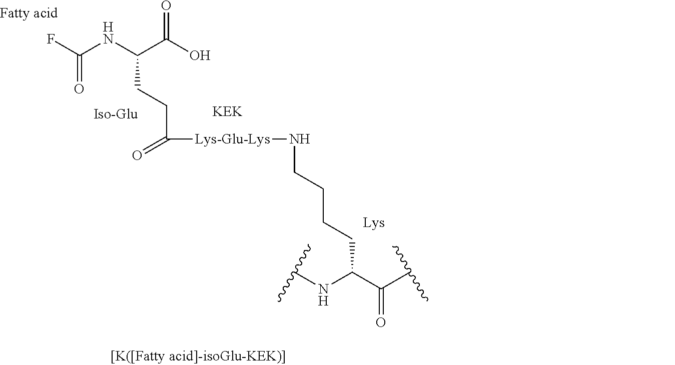

Furthermore, the substituent [Hexadecanoyl]-isoGlu, conjugated to the side chain of a lysine residue, is illustrated below:

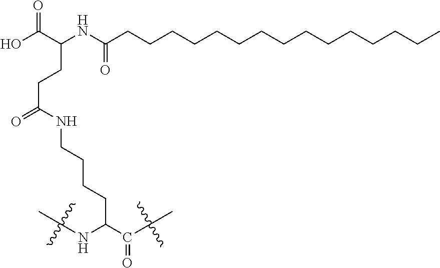 [SMILES: *NC(CCCCNC(=O)CCC(NC(=O)CCCCCCCCCCCCCCC)C(=O)O)C(*)=O]

Thus, the side chain of the Lys residue is covalently attached to the side-chain carboxyl group of the isoGlu spacer —Z²— (—Z¹—) via an amide linkage. A hexadecanoyl group (Z′) is covalently attached to the amino group of the isoGlu spacer via an amide linkage.

The substituent [Hexadecanoyl]-[4-aminobutanoyl]- conjugated to the side chain of a lysine residue, is illustrated below

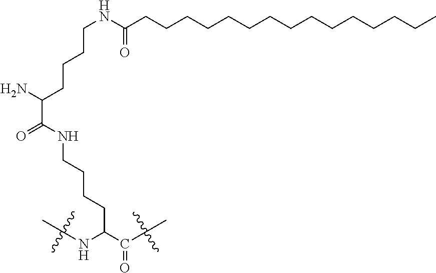 [SMILES: *NC(CCCCNC(=O)C(N)CCCCNC(=O)CCCCCCCCCCCCCCC)C(*)=O]

The substituent [(Hexadecanoyl)iso-Lys]- conjugated to the side chain of a lysine residue, is illustrated below

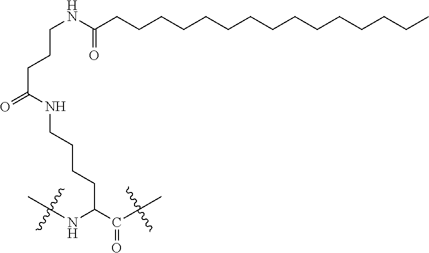 [SMILES: *NC(CCCCNC(=O)CCCNC(=O)CCCCCCCCCCCCCCC)C(*)=O]

The substituent [(Hexadecanoyl)β-Ala]- conjugated to the side chain of a lysine residue, is illustrated below

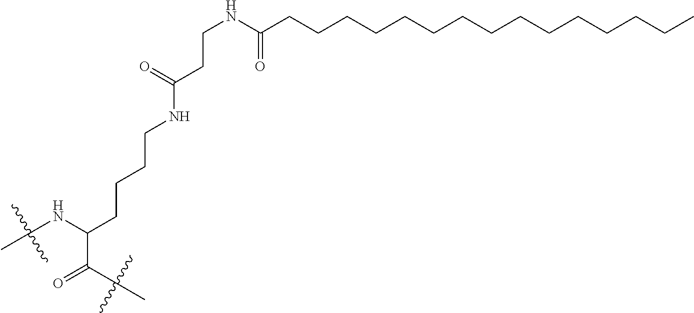 [SMILES: *NC(CCCCNC(=O)CCNC(=O)CCCCCCCCCCCCCCC)C(*)=O]

Some further specific examples of —Z²—Z¹ combinations are illustrated below. In each case, --- indicates the point of attachment to the side chain of the amino acid component of Ψ:

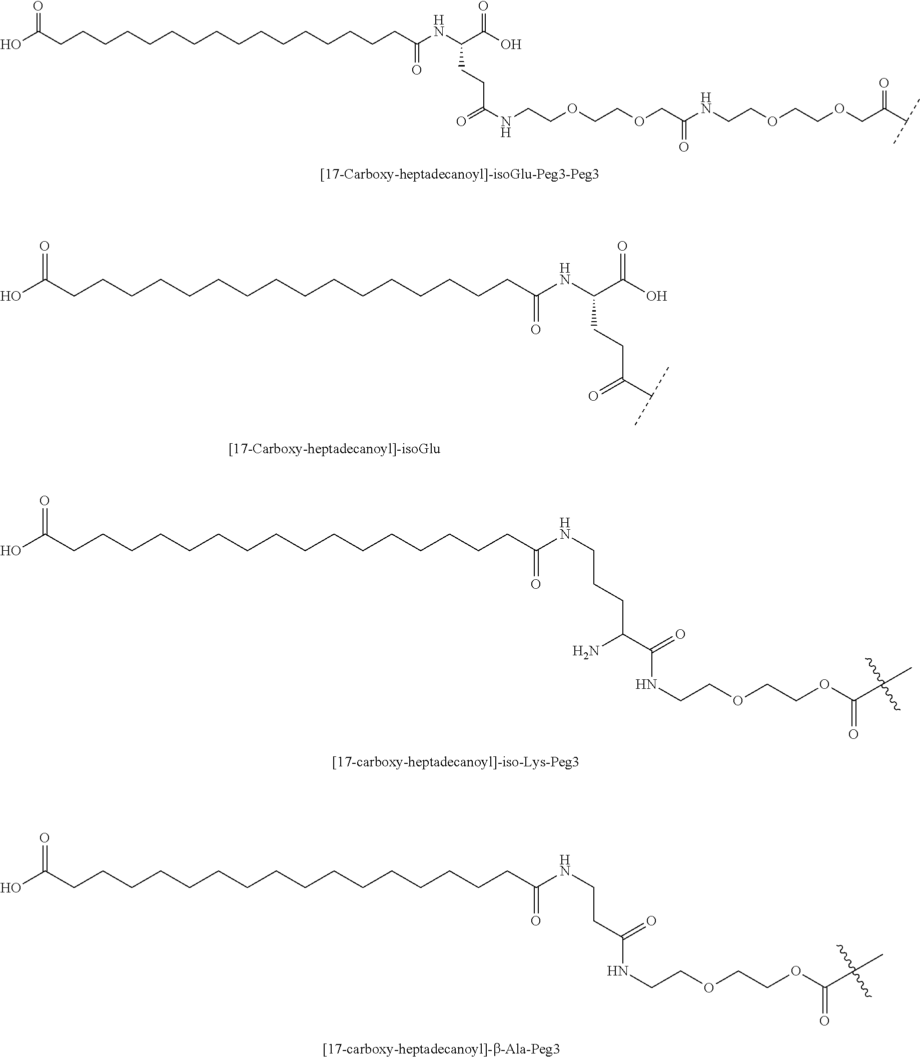

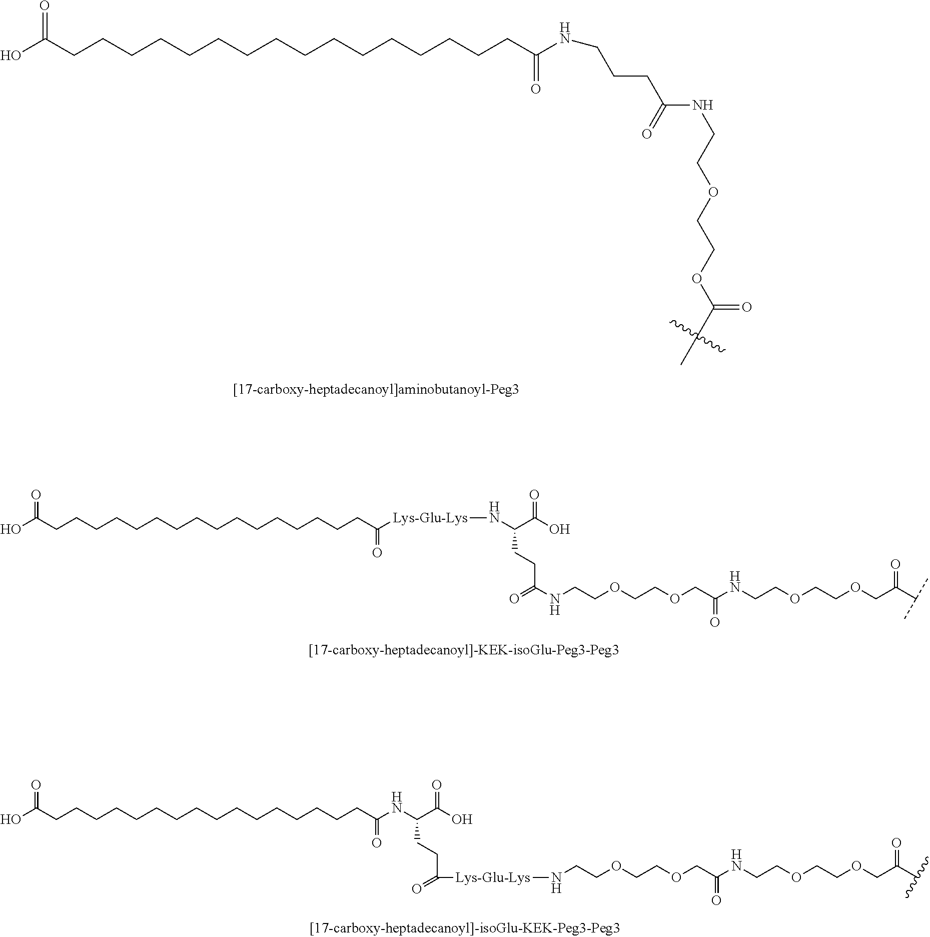

The skilled person will be well aware of suitable techniques for preparing the substituents employed in the context of the invention and conjugating them to the side chain of the appropriate amino acid in the dual agonist peptide. For examples of suitable chemistry, see WO98/08871, WO00/55184, WO00/55119, Madsen et al., J. Med. Chem. 50:6126-32 (2007), and Knudsen et al., J. Med Chem. 43:1664-1669 (2000), incorporated herein by reference.

### Synthesis of Dual Agonists

It is preferred to synthesize dual agonists of the invention by means of solid-phase or liquid-phase peptide synthesis methodology. In this context, reference may be made to WO 98/11125 and, among many others, Fields, G. B. et al., 2002, “Principles and practice of solid-phase peptide synthesis”. In: Synthetic Peptides (2nd Edition), and the Examples herein.

In accordance with the present invention, a dual agonist of the invention may be synthesized or produced in a number of ways, including for example, a method which comprises
(a) synthesizing the dual agonist by means of solid-phase or liquid-phase peptide synthesis methodology and recovering the synthesized dual agonist thus obtained; or
(b) expressing a precursor peptide sequence from a nucleic acid construct that encodes the precursor peptide, recovering the expression product, and modifying the precursor peptide to yield a compound of the invention.

The precursor peptide may be modified by introduction of one or more non-proteinogenic amino acids, e.g. Aib, Orn, Dap, or Dab, introduction of a lipophilic substituent Z¹ or Z¹—Z²— at a residue Ψ, introduction of the appropriate terminal groups R¹ and R², etc.

Expression is typically performed from a nucleic acid encoding the precursor peptide, which may be performed in a cell or a cell-free expression system comprising such a nucleic acid. It is preferred to synthesize the analogues of the invention by means of solid-phase or liquid-phase peptide synthesis. In this context, reference is made to WO 98/11125 and, among many others, Fields, G B et al., 2002, “Principles and practice of solid-phase peptide synthesis”. In: Synthetic Peptides (2nd Edition), and the Examples herein.

For recombinant expression, the nucleic acid fragments encoding the precursor peptide will normally be inserted in suitable vectors to form cloning or expression vectors. The vectors can, depending on purpose and type of application, be in the form of plasmids, phages, cosmids, mini-chromosomes, or virus, but also naked DNA which is only expressed transiently in certain cells is an important vector. Preferred cloning and expression vectors (plasmid vectors) are capable of autonomous replication, thereby enabling high copy-numbers for the purposes of high-level expression or high-level replication for subsequent cloning.

In general outline, an expression vector comprises the following features in the 5′→3′ direction and in operable linkage: a promoter for driving expression of the nucleic acid fragment, optionally a nucleic acid sequence encoding a leader peptide enabling secretion (to the extracellular phase or, where applicable, into the periplasma), the nucleic acid fragment encoding the precursor peptide, and optionally a nucleic acid sequence encoding a terminator. They may comprise additional features such as selectable markers and origins of replication. When operating with expression vectors in producer strains or cell lines it may be preferred that the vector is capable of integrating into the host cell genome. The skilled person is very familiar with suitable vectors and is able to design one according to their specific requirements.

The vectors of the invention are used to transform host cells to produce the precursor peptide. Such transformed cells can be cultured cells or cell lines used for propagation of the nucleic acid fragments and vectors, and/or used for recombinant production of the precursor peptides.

Preferred transformed cells are micro-organisms such as bacteria [such as the species Escherichia (e.g. E. coli), Bacillus (e.g. Bacillus subtilis), Salmonella, or Mycobacterium (preferably non-pathogenic, e.g. M. bovis BCG), yeasts (e.g., Saccharomyces cerevisiae and Pichia pastoris), and protozoans. Alternatively, the transformed cells may be derived from a multicellular organism, i.e. it may be fungal cell, an insect cell, an algal cell, a plant cell, or an animal cell such as a mammalian cell. For the purposes of cloning and/or optimised expression it is preferred that the transformed cell is capable of replicating the nucleic acid fragment of the invention. Cells expressing the nucleic fragment can be used for small-scale or large-scale preparation of the peptides of the invention.

When producing the precursor peptide by means of transformed cells, it is convenient, although far from essential, that the expression product is secreted into the culture medium.

### Pharmaceutical Compositions

An aspect of the present invention relates to a composition comprising a dual agonist according to the invention, or a pharmaceutically acceptable salt or solvate thereof, together with a carrier. In one embodiment of the invention, the composition is a pharmaceutical composition and the carrier is a pharmaceutically acceptable carrier. The present invention also relates to a pharmaceutical composition comprising a dual agonist according to the invention, or a salt or solvate thereof, together with a carrier, excipient or vehicle. Accordingly, the dual agonist of the present invention, or salts or solvates thereof, especially pharmaceutically acceptable salts or solvates thereof, may be formulated as compositions or pharmaceutical compositions prepared for storage or administration, and which comprise a therapeutically effective amount of a dual agonist of the present invention, or a salt or solvate thereof.

Suitable salts formed with bases include metal salts, such as alkali metal or alkaline earth metal salts, for example sodium, potassium or magnesium salts; ammonia salts and organic amine salts, such as those formed with morpholine, thiomorpholine, piperidine, pyrrolidine, a lower mono-, di- or tri-alkylamine (e.g., ethyl-tert-butyl-, diethyl-, diisopropyl-, triethyl-, tributyl- or dimethylpropylamine), or a lower mono-, di- or tri-(hydroxyalkyl)amine (e.g., mono-, di- or triethanolamine). Internal salts may also be formed. Similarly, when a compound of the present invention contains a basic moiety, salts can be formed using organic or inorganic acids. For example, salts can be formed from the following acids: formic, acetic, propionic, butyric, valeric, caproic, oxalic, lactic, citric, tartaric, succinic, fumaric, maleic, malonic, mandelic, malic, phthalic, hydrochloric, hydrobromic, phosphoric, nitric, sulphuric, benzoic, carbonic, uric, methanesulphonic, naphthalenesulphonic, benzenesulphonic, toluenesulphonic, p-toluenesulphonic (i.e. 4-methylbenzene-sulphonic), camphorsulphonic, 2— aminoethanesulphonic, aminomethylphosphonic and trifluoromethanesulphonic acid (the latter also being denoted triflic acid), as well as other known pharmaceutically acceptable acids.

Amino acid addition salts can also be formed with amino acids, such as lysine, glycine, or phenylalanine.

In one embodiment, a pharmaceutical composition of the invention is one wherein the dual agonist is in the form of a pharmaceutically acceptable acid addition salt.

In some embodiments, the pharmaceutical composition of the invention is formulated as 1 mL solution for injection.

### Titration Dose and Treatment Dose

The dose may be a titration dose or a treatment dose.

The term “titration dose” refers to the dose of the dual agonist administered to the patient at each administration during the titration period, prior to administrations at the treatment dose. Each titration dose is in an amount of 0.1 mg to 10.0 mg of the dual agonist. The doses, dosage regime and administration protocols presented herein equally apply to the titration dose(s).

The term “treatment dose” refers to the dose of the dual agonist administered to the patient at each administration during the treatment period. Each treatment dose is in an amount of 0.1 mg to 10.0 mg of the dual agonist. The doses, dosage regime and administration protocols presented herein equally apply to the treatment dose(s).

### Dosage Regime

According to the invention, the dual GLP-1/GLP-2 agonists are for use in a method of reducing or inhibiting weight gain, reducing food intake, reducing appetite, promoting weight loss, or treating obesity, morbid obesity, obesity-linked gallbladder disease, or obesity-induced sleep apnea wherein the method comprises administering the dual agonist to the patient at a dose of about 0.1 mg to 10.0 mg. In other words, the method comprises administering the dual agonist to the patient in an amount of about 0.1 mg to 10.0 mg.

The dose of about 0.1 mg to 10.0 mg of dual agonist is administered to the patient in a single administration (i.e. a single administration event). In other words, the dual agonist is administered to the patient in a single dosage formulation of about 0.1 mg to about 10.0 mg. This single dosage formulation may be administered to the patient once or multiple times wherein each of the multiple dosage formulations for administration to the patient need not comprise the same amount of the dual agonist. In other words, the dual agonist may be administered to the patient in a series of single administrations wherein each of the single administrations may not comprise the same amount of the dual agonist. Each administration of the dual agonist to the patient may be independently selected to be at a dose of about 0.1 mg to about 10.0 mg.

Thus, the invention provides a GLP-1/GLP-2 dual agonist as described herein, or a pharmaceutically acceptable salt or solvate thereof, for use in a method of reducing or inhibiting weight gain, reducing food intake, reducing appetite, promoting weight loss, or treating obesity, morbid obesity, obesity-linked gallbladder disease, or obesity-induced sleep apnea, wherein the method comprises at least one administration of the dual agonist to the patient at a dose of about 0.1 mg to 10.0 mg.

### Dose

In one aspect the dual agonist is administered to the patient at a dose of from about 0.1 mg to about 10.0 mg. In one aspect the dual agonist is administered to the patient at a dose of from about 1.0 mg to about 10.0 mg. In one aspect the dual agonist is administered to the patient at a dose of from about 1.1 mg to about 10.0 mg, from about 1.2 mg to about 10.0 mg, from about 1.3 mg to about 10.0 mg, or from about 1.4 mg to about 10.0 mg. In one aspect the dual agonist is administered to the patient at a dose of from about 1.5 mg to about 10.0 mg. In one aspect the dual agonist is administered to the patient at a dose of from about 1.6 mg to about 10.0 mg, from about 1.7 mg to about 10.0 mg, from about 1.8 mg to about 10.0 mg, or from about 1.9 mg to about 10.0 mg. In one aspect the dual agonist is administered to the patient at a dose of from about 2.0 mg to about 10.0 mg. In one aspect the dual agonist is administered to the patient at a dose of from about 2.1 mg to about 10.0 mg, or from about 2.2 mg to about 10.0 mg. In one aspect the dual agonist is administered to the patient at a dose of from about 2.25 mg to about 10.0 mg. In one aspect the dual agonist is administered to the patient at a dose of from about 3.0 mg to about 10.0 mg, from about 4.0 mg to about 10.0 mg, from about 5.0 mg to about 10.0 mg, from about 6.0 mg to about 10.0 mg, from about 7.0 mg to about 10.0 mg, from about 8.0 mg to about 10.0 mg, or from about 9.0 mg to about 10.0 mg.

In one aspect the dual agonist is administered to the patient at a dose of from about 0.1 mg to about 9.0 mg. In one aspect the dual agonist is administered to the patient at a dose of from about 1.0 mg to about 9.0 mg. In one aspect the dual agonist is administered to the patient at a dose of from about 1.1 mg to about 9.0 mg, from about 1.2 mg to about 9.0 mg, from about 1.3 mg to about 9.0 mg, or from about 1.4 mg to about 9.0 mg. In one aspect the dual agonist is administered to the patient at a dose of from about 1.5 mg to about 9.0 mg. In one aspect the dual agonist is administered to the patient at a dose of from about 1.6 mg to about 9.0 mg, from about 1.7 mg to about 9.0 mg, from about 1.8 mg to about 9.0 mg, or from about 1.9 mg to about 9.0 mg. In one aspect the dual agonist is administered to the patient at a dose of from about 2.0 mg to about 9.0 mg. In one aspect the dual agonist is administered to the patient at a dose of from about 2.1 mg to about 9.0 mg, or from about 2.2 mg to about 9.0 mg. In one aspect the dual agonist is administered to the patient at a dose of from about 2.25 mg to about 9.0 mg. In one aspect the dual agonist is administered to the patient at a dose of from about 3.0 mg to about 9.0 mg, from about 4.0 mg to about 9.0 mg, from about 5.0 mg to about 9.0 mg, from about 6.0 mg to about 9.0 mg, from about 7.0 mg to about 9.0 mg, or from about 8.0 mg to about 9.0 mg.

In one aspect the dual agonist is administered to the patient at a dose of from about 0.1 mg to about 8.0 mg. In one aspect the dual agonist is administered to the patient at a dose of about 1.0 mg to about 8.0 mg. In one aspect the dual agonist is administered to the patient at a dose of about 1.1 mg to about 8.0 mg, about 1.2 mg to about 8.0 mg, about 1.3 mg to about 8.0 mg, or about 1.4 mg to about 8.0 mg. In one aspect the dual agonist is administered to the patient at a dose of about 1.5 mg to about 8.0 mg. In one aspect the dual agonist is administered to the patient at a dose of from about 1.6 mg to about 8.0 mg, from about 1.7 mg to about 8.0 mg, from about 1.8 mg to about 8.0 mg, or from about 1.9 mg to about 8.0 mg. In one aspect the dual agonist is administered to the patient at a dose of from about 2.0 mg to about 8.0 mg. In one aspect the dual agonist is administered to the patient at a dose of from about 2.1 mg to about 8.0 mg, or from about 2.2 mg to about 8.0 mg. In one aspect the dual agonist is administered to the patient at a dose of from about 2.25 mg to about 8.0 mg. In one aspect the dual agonist is administered to the patient at a dose of from about 3.0 mg to about 8.0 mg, from about 4.0 mg to about 8.0 mg, from about 5.0 mg to about 8.0 mg, from about 6.0 mg to about 8.0 mg, or from about 7.0 mg to about 8.0 mg.

In one aspect the dual agonist is administered to the patient at a dose of from about 1.0 mg to about 7.5 mg, from about 1.0 mg to about 7.0 mg, from about 1.0 mg to about 6.0 mg, from about 1.0 mg to about 5.0 mg, from about 1.0 mg to about 4.0 mg, or from about 1.0 mg to about 3.5 mg. In one aspect the dual agonist is administered to the patient at a dose of about 1.5 mg to about 7.5 mg. In one aspect the dual agonist is administered to the patient at a dose of from about 1.5 mg to about 7.0 mg, from about 1.5 mg to about 6.0 mg, from about 1.5 mg to about 5.0 mg, from about 1.5 mg to about 4.0 mg, or from about 1.5 mg to about 3.5 mg. In one aspect the dual agonist is administered to the patient at a dose from about 2.0 mg to about 7.5 mg, from about 2.0 mg to about 7.0 mg, from about 2.0 mg to about 6.0 mg, from about 2.0 mg to about 5.0 mg, from about 2.0 mg to about 4.0 mg, or from about 2.0 mg to about 3.5 mg. In one aspect the dual agonist is administered to the patient at a dose of from about 2.25 mg to about 7.5 mg, from about 2.25 mg to about 7.0 mg, from about 2.25 mg to about 6.0 mg, from about 2.25 mg to about 5.0 mg, from about 2.25 mg to about 4.0 mg, or from about 2.25 mg to about 3.5 mg. In one aspect the dual agonist is administered to the patient at a dose of from about 4.0 mg to about 7.5 mg. In one aspect the dual agonist is administered to the patient at a dose of from about 4.0 mg to about 6.0 mg.

In one aspect the dual agonist is administered to the patient at a dose of from 1.0 mg to 7.5 mg, from 1.0 mg to 7.0 mg, from 1.0 mg to 6.0 mg, from 1.0 mg to 5.0 mg, from 1.0 mg to 4.0 mg, or from 1.0 mg to 3.5 mg. In one aspect the dual agonist is administered to the patient at a dose of 1.5 mg to 7.5 mg. In one aspect the dual agonist is administered to the patient at a dose of from 1.5 mg to 7.0 mg, from 1.5 mg to 6.0 mg, from 1.5 mg to 5.0 mg, from 1.5 mg to 4.0 mg, or from 1.5 mg to 3.5 mg. In one aspect the dual agonist is administered to the patient at a dose from 2.0 mg to 7.5 mg, from 2.0 mg to 7.0 mg, from 2.0 mg to 6.0 mg, from 2.0 mg to 5.0 mg, from 2.0 mg to 4.0 mg, or from 2.0 mg to 3.5 mg. In one aspect the dual agonist is administered to the patient at a dose of from 2.25 mg to 7.5 mg, from 2.25 mg to 7.0 mg, from 2.25 mg to 6.0 mg, from 2.25 mg to 5.0 mg, from 2.25 mg to 4.0 mg, or from 2.25 mg to 3.5 mg. In one aspect the dual agonist is administered to the patient at a dose of from 4.0 mg to 7.5 mg. In one aspect the dual agonist is administered to the patient at a dose of from 4.0 mg to 6.0 mg.

In one aspect the dose is more than 0.6 mg. In one aspect the dual agonist is administered to the patient at a dose of about 1.5 mg.

In some aspects the dual agonist is administered to the patient at a dose of about 1.0 mg, about 1.5 mg, about 2.0 mg, about 2.25 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 9.0 mg or about 10.0 mg. In some aspects the dual agonist is administered to the patient at a dose of 1.0 mg, 1.5 mg, 2.0 mg, 2.25 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 9.0 mg or 10.0 mg.

### Administration

The administration of the dual agonists described herein may be by any mode of administration common or standard in the art, e.g. oral, intravenous, intramuscular, subcutaneous, sublingual, intranasal, intradermal, suppository routes or implanting. In a preferred embodiment of the invention as described herein administration is by subcutaneous injection.

The dosage regime of the invention may involve administering more than one dose of the dual agonist. Thus, in some aspects the invention provides a GLP-1/GLP-2 dual agonist as described herein, or a pharmaceutically acceptable salt or solvate thereof, for use in a method of reducing or inhibiting weight gain, reducing food intake, reducing appetite, promoting weight loss, or treating obesity, morbid obesity, obesity-linked gallbladder disease, or obesity-induced sleep apnea, wherein the method comprises one or more administrations of the dual agonist to the patient at a dose of about 0.1 mg to 10.0 mg. In some aspects, the method comprises two or more administrations of the dual agonist to the patient at a dose of about 0.1 mg to 10.0 mg. In some aspects, each administration of the dual agonist to the patient is at a dose of about 0.1 mg to 10.0 mg.

In some aspects of the invention wherein the method comprises more than one administration of the dual agonist to the patient, the dose of the dual agonist may be different at each administration. In other words, it is not required that the dose of the dual agonist is the same at each administration. However, in other aspects of the invention wherein the method comprises more than one administration of the dual agonist to the patient, the dose of the dual agonist may be the same, or substantially the same, at each administration.

For some aspects of the present invention, a series of single administrations are delivered to the patient wherein the initial course of the single administrations may have subsequent increasing dose amounts of the dual agonist in the single dosage formulations. In some aspects, the initial course may include any one of 2, 3, 4, 5, 6, 7, 8, 9, 10 or more administrations of increasing amounts of the dual agonist in the single administration formulations. In some aspects, after the initial course the subsequent dose amounts of the dual agonist in the single dosage formulations may be the same as the last dose of the initial course or may be less than the dose of the last dose of the initial course or may be higher than the last dose of the initial course. In certain aspects, after the initial course the subsequent dose amounts of the dual agonist in the single dosage formulations may be the same or about the same as the last dose of the initial course.

In a preferred embodiment, the administration involves weekly administration of the dual agonist.

The reference to “weekly” is intended to mean approximately every 7 days, for example, approximately every 5, 5.5, 6, 6.5, 7, 7.5, 8, 8.5 or 9 days with each “day” being counted as approximately a 24 hour period. As will be appreciated in the art, the time between doses may be varied to some extent so that each and every dose is not separated by precisely the same time. This will often be directed under the discretion of the physician. Thus, doses may be separated in time by a clinically acceptable range of times.

In one aspect of the invention described herein the reference to “weekly” may mean 7 days±2 days. That is to say the administration may take place either up to and including two days before, or up to and including two days after the stated day. As such, the administration may take place 2 or 1 days before, or 1 or 2 days after, the stated day.

In one aspect, the dual agonist is administered weekly at a dose of from about 1.5 mg to about 7.5 mg, such as from about 1.5 mg to about 6.0 mg, such as from about 1.5 mg to about 4.0 mg, such as from about 1.5 mg to about 3.5 mg. In one aspect, the dual agonist is administered weekly at a dose of from about 2.0 mg to about 7.5 mg, such as from about 2.0 mg to about 6.0 mg, such as from about 2.0 mg to about 4.0 mg, such as from about 2.0 mg to about 3.5 mg. In one aspect, the dual agonist is administered weekly at a dose of from about 2.25 mg to about 3.5 mg.

In one aspect, the dual agonist is administered weekly at a dose of from 1.5 mg to 7.5 mg, such as from 1.5 mg to 6.0 mg, such as from 1.5 mg to 4.0 mg, such as from 1.5 mg to 3.5 mg. In one aspect, the dual agonist is administered weekly at a dose of from 2.0 mg to 7.5 mg, such as from 2.0 mg to 6.0 mg, such as from 2.0 mg to 4.0 mg, such as from 2.0 mg to 3.5 mg. In one aspect, the dual agonist is administered weekly at a dose of from 2.25 mg to 3.5 mg.

In one aspect the number of doses administered to a patient may be 1, 2, 3, 4, 5, 6, 7, 8, 9, 10, 11, or 12 or more doses. In other words, in some aspects the dual agonist is administered to the patient 1, 2, 3, 4, 5, 6, 7, 8, 9, 10, 11, or 12 or more times. In some aspects, the method comprises 1, 2, 3, 4, 5, 6, 7, 8, 9, 10, 11, or 12 or more administrations of the dual agonist. In some aspects the dual agonist is administered to the patient 1, 2, 3, 4, 5, 6, 7, 8, 9, 10, 11, or 12 or more times at a dose of about 0.1 to 10.0 mg (or at any other dose described herein). In some aspects, the method comprises 1, 2, 3, 4, 5, 6, 7, 8, 9, 10, 11, or 12 or more administrations of the dual agonist at a dose of about 0.1 to 10.0 mg (or at any other dose described herein). In one aspect, 4 doses are administered to a patient. In one aspect, the method comprises 4 administrations of the dual agonist at a dose of about 0.1 to 10.0 mg (or at any other dose described herein). In one aspect, 12 doses are administered to a patient. In one aspect, the method comprises 12 administrations of the dual agonist at a dose of about 0.1 to 10.0 mg (or at any other dose described herein).

In one aspect the agonist may be administered at the same dose each time. In one aspect, each administration to the patient of the dual agonist is at a dose of about 0.1 mg to 10.0 mg.

In one aspect a number of doses are administered to a patient over a period of weeks, or months or for 1 year or more than 1 year.

In one aspect a number of doses are administered to a patient weekly and over a period of weeks, or months or for 1 year or more than 1 year.

In one aspect the agonist may be administered in ascending doses.

### Titration and Treatment

In some aspects, the dual agonist is administered to the patient according to a titration regimen. A titration regimen comprises an initial set of one or more administrations of the dual agonist in a “titration period” followed by a set of one or more administrations of the dual agonist in a “treatment period”. Typically, the dose of the dual agonist at each administration in the titration period is lower than the dose at each administration in the treatment period.

A first purpose of the titration period is to acclimatize the patient to side-effects of the dual agonist. Initial administration of the dual agonist may produce side-effects which decrease in severity after further administrations as the patient adapts. Administering the dual agonist at a lower dose in the titration period may curtail the initial severity of these side-effects. A second purpose of the titration period may be to determine an appropriate dose for the dual agonist for the patient. The dose of the dual agonist may be increased across the titration period, allowing a physician to observe side-effects at different doses and thereby determine an appropriate dose for treatment.

Thus, in some aspects the invention provides a GLP-1/GLP-2 dual agonist as described herein, or a pharmaceutically acceptable salt or solvate thereof, for use in a method of reducing or inhibiting weight gain, reducing food intake, reducing appetite, promoting weight loss, or treating obesity, morbid obesity, obesity-linked gallbladder disease, or obesity-induced sleep apnea, wherein the method comprises at least one administration of the dual agonist to the patient at a dose of about 0.1 mg to 10.0 mg, and wherein the method comprises at least one administration of the dual agonist to the patient at a titration dose and at least one administration of the dual agonist to the patient at a treatment dose. In other words, in some aspects the method comprises administering the dual agonist to the patient at least once at a titration dose and at least once at a treatment dose.

In some aspects, the method comprises more than one administration (i.e. 2 or more administrations) of the dual agonist to the patient at a titration dose. In some aspects, the method comprises 3 or more, 4 or more or 5 or more administrations of the dual agonist to the patient at a titration dose. In some aspects, the method comprises 1, 2, 3, 4 or 5 administrations of the dual agonist to the patient at a titration dose. In preferred aspects, the method comprises 2 administrations of the dual agonist to the patient at a titration dose. In preferred aspects, the method comprises 5 administrations of the dual agonist to the patient at a titration dose.

In one aspect there may be at least one initial titration period of a lower dose prior to increasing the dose. In one aspect the titration period may constitute 1, 2, 3, or 4 doses of a lower dose, wherein preferably the doses are the same each time. In one aspect the titration period consists of 1 dose of a lower dose. In one aspect the titration period consists of 2 doses of a lower dose.

In a preferred aspect the titration doses are administered weekly. In other words, in some aspects the method comprises administering the dual agonist to the patient once weekly at a titration dose.

The titration dose may be any dose of dual agonist as described elsewhere herein. In some aspects titration dose is from about 0.1 mg to about 10.0 mg. In some aspects the titration dose is from about 1.0 mg to about 6.0 mg, for example from about 1.5 mg to about 6.0 mg. Thus, in some aspects the method comprises at least one administration of the dual agonist to the patient at a titration dose of from about 1.5 mg to about 6.0 mg. In one aspect the titration dose is from about 1.0 mg to about 4.0 mg, for example from about 1.5 mg to about 4.0 mg. In one aspect the titration dose is about 1.0 mg to about 3.5 mg, for example about 1.5 mg to about 3.5 mg, or about 1.5 mg to about 3.0 mg. In one aspect the titration dose is or is about 1.0 mg, 2.0 mg, 2.25 mg, 3.0 mg, 3.5 mg, 4.0 mg, 4.5 mg, 5.0 mg, 5.5 mg or 6.0 mg. In some aspects the titration dose is 2.0 mg. In some aspects the titration dose is 2.0 mg administered once weekly. In some aspects the titration dose is 4.0 mg. In some aspects the titration dose is 4.0 mg administered once weekly.

The titration dose is not required to be the same at each administration. In other words, different titration doses may be administered to the patient within the titration period. Thus, in some aspects the invention provides a GLP-1/GLP-2 dual agonist as described herein, or a pharmaceutically acceptable salt or solvate thereof, for use in a method of reducing or inhibiting weight gain, reducing food intake, reducing appetite, promoting weight loss, or treating obesity, morbid obesity, obesity-linked gallbladder disease, or obesity-induced sleep apnea, wherein the method comprises at least one administration of the dual agonist to the patient at a dose of about 0.1 mg to 10.0 mg, and wherein the method comprises at least one administration of the dual agonist to the patient at one or more titration doses and at least one administration of the dual agonist to the patient at a treatment dose.

In some aspects, the method comprises 2 or more, 3 or more, or 4 or more different titration doses. In some aspects, the method comprises 2, 3, or 4 different titration doses. In preferred aspects, the method comprises 2 different titration doses. Each titration dose may be any of the doses of dual agonist described elsewhere herein.

In some aspects, all titration doses are the same (i.e. there is one titration dose, which is the same for all administrations of the dual agonist to the patient in the titration period).

In some aspects, the method comprises one administration of the dual agonist to the patient at a titration dose of 3.5 mg. In some aspects, the method comprises two administrations of the dual agonist to the patient at a titration dose of 2.0 mg. In some aspects, the method comprises two administrations of the dual agonist to the patient at a titration dose of 2.0 mg and three administrations of the dual agonist to the patient at a titration dose of 4.0 mg.

In some aspects, the method comprises more than one administration (i.e. 2 or more administrations) of the dual agonist to the patient at a treatment dose. In some aspects, the method comprises 3 or more, 4 or more, 5 or more, 6 or more, 7 or more, 8 or more, 9 or more, 10 or more, 11 or more, or 12 or more administrations of the dual agonist to the patient at a treatment dose. In some aspects, the method comprises 1, 2, 3, 4, 5, 6, 7, 8, 9, 10, 11 or 12 administrations of the dual agonist to the patient at a treatment dose. In preferred aspects, the method comprises 3 administrations of the dual agonist to the patient at a treatment dose. In preferred aspects, the method comprises 10 administrations of the dual agonist to the patient at a treatment dose. In preferred aspects, the method comprises 7 administrations of the dual agonist to the patient at a treatment dose.

The treatment dose may continue to be administered for as long as is necessary. The dual agonist at a treatment dose may be administered to the patient for a period of, for example, one month to twenty years, for example for a period of one month, two months, three months, four months, five months, six months, seven months, eight months, nine months, ten months, eleven months, one year, two years, three years, four years, five years, six years, seven years, eight years, nine years, ten years, eleven years, twelve years, thirteen years, fourteen years, fifteen years, sixteen years, seventeen years, eighteen years, nineteen years or twenty years.

For example, the treatment dose may be administered weekly to the patient for a period of, for example, one month to twenty years, for example for a period of one month, two months, three months, four months, five months, six months, seven months, eight months, nine months, ten months, eleven months, one year, two years, three years, four years, five years, six years, seven years, eight years, nine years, ten years, eleven years, twelve years, thirteen years, fourteen years, fifteen years, sixteen years, seventeen years, eighteen years, nineteen years or twenty years.

The treatment dose may be any dose of dual agonist described herein. In some aspects, the treatment dose is from about 0.1 mg to about 10.0 mg. In some aspects the treatment dose is from about 1.0 mg to about 10.0 mg, from about 1.5 mg to about 10.0 mg, from about 2.0 mg to about 10.0 mg, from about 2.25 mg to about 10.0 mg, from about 3.0 mg to about 10.0 mg, from about 4.0 mg to about 10.0 mg, from about 5.0 mg to about 10.0 mg, from about 6.0 mg to about 10.0 mg, from about 7.0 mg to about 10.0 mg, from about 8.0 mg to about 10.0 mg, or from about 9.0 mg to about 10.0 mg. In some aspects the treatment dose is about 1.0 mg, about 1.5 mg, about 2.0 mg, about 2.25 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 9.0 mg or about 10.0 mg. In some aspects the treatment dose is 1.0 mg, 1.5 mg, 2.0 mg, 2.25 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 9.0 mg or 10.0 mg.

Typically, in aspects wherein the dual agonist is administered to the patient at the treatment dose more than once (i.e. multiple administrations of the dual agonist at the treatment dose) all administrations in the treatment period are at the same dose. Thus, in some aspects, all treatment doses are the same (i.e. there is one treatment dose, which is the same for all administrations of the dual agonist to the patient in the treatment period).

However, the treatment dose is not required to be the same at each administration. In other words, different treatment doses may be administered to the patient within the treatment period. The treatment dose may be varied in accordance with the patient's response to the dual agonist. For example, if the patient develops severe side-effects at a given treatment dose, the treatment dose may be lowered at future administrations to reduce the severity of the side-effects.

Thus, in some aspects the invention provides a GLP-1/GLP-2 dual agonist as described herein, or a pharmaceutically acceptable salt or solvate thereof, for use in a method of reducing or inhibiting weight gain, reducing food intake, reducing appetite, promoting weight loss, or treating obesity, morbid obesity, obesity-linked gallbladder disease, or obesity-induced sleep apnea, wherein the method comprises at least one administration of the dual agonist to the patient at a dose of about 0.1 mg to 10.0 mg, and wherein the method comprises at least one administration of the dual agonist to the patient at one or more titration doses and at least one administration of the dual agonist to the patient one or more treatment doses.

In some aspects, the method comprises 2 or more, 3 or more, or 4 or more different treatment doses. In some aspects, the method comprises 2, 3, or 4 different treatment doses. Each treatment dose may be any of the doses of dual agonist described elsewhere herein.

Typically, the treatment dose is higher than the titration dose. Thus, in some aspects, the treatment dose is higher than the titration dose. In some aspects, the treatment dose is higher than some or all of the titration doses.

However, the treatment dose may be lower than the titration dose. This may be the case, for example, where the titration dose is increased as the titration period progresses (i.e. the titration dose becomes higher over successive administrations) but then the dose is decreased for the treatment dose in view of side-effects experienced by the patient as the titration dose increased. Thus, in some aspects, the treatment dose is lower than the titration dose. In some aspects, the treatment dose is lower than some or all of the titration doses.

As described herein, a purpose of titration doses is to identify an appropriate treatment dose. Thus, in some aspects the treatment dose is determined by a physician observing the effects of the titration dose on patients. In other words, the treatment dose may depend on the titration dose.

In a preferred aspect the treatment doses are administered weekly. In other words, in some aspects the method comprises administering the dual agonist to the patient once weekly at a treatment dose. In some aspects, the method comprises administering the dual agonist to the patient once weekly at a titration dose and once weekly at a treatment dose. In other words, the once weekly administration of the dual agonist at the treatment dose is a continuation of the once weekly administrations at the titration dose.

In one aspect the titration period may be followed by one or more doses at a higher dose than the titration dose. In one aspect the titration period is followed by 1, 2, 3 or 4 doses at a higher dose than the titration dose. In one aspect the titration period is followed by 3 doses at a higher dose than the titration dose. In one aspect the titration period is followed by 10 doses at a higher dose than the titration dose. In one aspect the titration period is followed by 7 doses at a higher dose than the titration dose. In one aspect the titration period consists of 1 dose and is followed by 3 doses at a higher dose than the titration dose. In one aspect the titration period consists of 2 doses and is followed by 10 doses at a higher dose than the titration dose. In one aspect the higher dose is between about 3 mg to about 8 mg. In one aspect the higher dose is from about 3 mg to about 8 mg.

In some aspects, the method comprises one administration of the dual agonist to the patient at a titration dose of 3.5 mg and three administrations of the dual agonist to the patient at a treatment dose of 6.0 mg, wherein each administration is once weekly.

In some aspects, the method comprises two administrations of the dual agonist to the patient at a titration dose of 2.0 mg and ten administrations of the dual agonist to the patient at a treatment dose of 4.0 mg, wherein each administration is once weekly.

In some aspects, the method comprises two administrations of the dual agonist to the patient at a titration dose of 2.0 mg, three administrations of the dual agonist to the patient at a titration dose of 4.0 mg, and seven administrations of the dual agonist to the patient at a treatment dose of 6.0 mg, wherein each administration is once weekly.

In one aspect the higher dose (following the titration period) is or is about 6.0 mg, 7.0 mg, 7.5 mg or 8.0 mg, preferably 6.0 mg.

In a preferred aspect the titration doses are administered weekly.

In a preferred aspect the post-titration doses are administered weekly.

Advantageously, the subject may not experience nausea or vomiting (or other adverse gastrointestinal effects) during the titration period. This allows for a shorter or expedited titration period prior to administration of higher doses.

In one aspect there may be more than one titration period.

In one aspect of the invention further doses are administered after the doses discussed above, i.e. the subject may continue to receive doses after the initial doses discussed herein.

Additional dosing may be once weekly.

Administration of the dual agonist may continue as long as necessary.

Additional doses as described above may be administered as required for a period of, for example, one month to twenty years, for example for a period of one month, two months, three months, four months, five months, six months, seven months, eight months, nine months, ten months, eleven months, one year, two years, three years, four years, five years, six years, seven years, eight years, nine years, ten years, eleven years, twelve years, thirteen years, fourteen years, fifteen years, sixteen years, seventeen years, eighteen years, nineteen years or twenty years.

### Clinical Outcomes

In a preferred embodiment, the patient does not experience side-effects of nausea and/or vomiting following administration of the dual agonist.

In a preferred embodiment, the patient has decreased appetite. In a preferred embodiment, the patient has decreased appetite following administration of the dual agonist.

The term “appetite” refers to a patient's desire to consume food. The appetite of the patient may be determined by measuring how much food the patient consumes using techniques known in the art and described herein, such as the mixed meal test or standard meal test described in Example 6 herein. Thus, in some embodiments, appetite is measured using the mixed meal test. In some embodiments, appetite is measured using the standard meal test. In some embodiments, following administration of the dual agonist the appetite of the patient is reduced by at least 5%. In some embodiments, following administration of the dual agonist the appetite of the patient is reduced by at least 10%, at least 15%, at least 20%, at least 25%, at least 30%, at least 35%, at least 40%, at least 45%, at least 50%, at least 55% or at least 60%.

In a preferred embodiment, the patient has reduced food consumption following administration of the dual agonist. “Food consumption” is synonymous with “food intake”. Thus, in a preferred embodiment, the patient has reduced food intake following administration of the dual agonist. The term “food consumption” refers to the amount of food the patient consumes in a given setting or period, such as a single meal, over multiple meals or over a particular period. Food consumption of the patient may be measured by techniques known in the art and described herein, such as the mixed meal test or standard meal test described in Example 6 herein. Thus, in some embodiments, food consumption is measured using the mixed meal test. In some embodiments, food consumption is measured using the standard meal test. In some embodiments, following administration of the dual agonist the food consumption of the patient is reduced by at least 5%. In some embodiments, following administration of the dual agonist the food consumption of the patient is reduced by at least 10%, at least 15%, at least 20%, at least 25%, at least 30%, at least 35%, at least 40%, at least 45%, at least 50%, at least 55% or at least 60%. In some embodiments, following administration of the dual agonist the amount of food consumed by the patient is reduced to 95% or less of the amount of food consumed by the patient prior to administration of the dual agonist. In some embodiments, following administration of the dual agonist the amount of food consumed by the patient is reduced to 90% or less, 85% or less, 80% or less, 75% or less, 70% or less, 65% or less, 60% or less, 55% or less, 50% or less, 45% or less or 40% or less of the amount of food consumed by the patient prior to administration of the dual agonist. In a preferred embodiment, following administration of the dual agonist the amount of food consumed by the patient is reduced to 65% or less of the amount of food consumed by the patient prior to administration of the dual agonist.

In some embodiments, the patient has reduced body weight following administration of the dual agonist. In some embodiments, following administration of the dual agonist the body weight of the patient is reduced by at least 5%, at least 10%, at least 15%, at least 20%, at least 25%, at least 30%, at least 35%, at least 40%, at least 45%, at least 50%, at least 55% or at least 60%.

In some embodiments, the patient has reduced body mass index (BMI) following administration of the dual agonist. In some embodiments, following administration of the dual agonist the BMI of the patient is reduced by at least 5%, at least 10%, at least 15%, at least 20%, at least 25%, at least 30%, at least 35%, at least 40%, at least 45%, at least 50%, at least 55% or at least 60%. BMI of a patient may be determined by methods known in the art.

### Tonicity Agent

In one aspect the composition or pharmaceutical composition may be an isotonic parenteral composition.

In one aspect the composition or pharmaceutical composition comprises a tonicity agent, for example as described in WO2020/249778. The isotonic parenteral pharmaceutical composition may comprise a GLP-1/GLP-2 dual agonist as described herein and:
a. about 5 mM to about 50 mM of phosphate buffer component, preferably about 10 mM to about 40 mM, more preferably about 15 mM to about 30 mM, and most preferably about 20 mM of phosphate buffer component; and
b. about 190 mM to about 240 mM of one or more tonicity agent, wherein said one or more tonicity agent comprises or is, preferably is, a non-ionic tonicity agent, and wherein the non-ionic tonicity agent is mannitol, wherein said composition further comprises a solvent, and wherein said composition has a pH of about pH 6.0 to about pH 8.2, preferably a pH of about pH 7.0 to about pH 8.0. The mannitol is preferably D-mannitol.

In one aspect the GLP-1/GLP-2 dual agonist comprises the sequence: H[Aib]EGSFTSELATILD[Ψ]QAARDFIAWLIQHKITD (SEQ ID NO 34), more preferably comprises

a.
Hy-H[Aib]EGSFTSELATILD[K([17-carboxy-
heptadecanoyl]-isoGlu)]QAARDFIAWLIQHKITD-OH
(CPD1OH);
or
b.
Hy-H[Aib]EGSFTSELATILD[K([17-carboxy-
heptadecanoyl]-isoGlu)]QAARDFIAWLIQHKITD-NH2
(CPD1NH2).

In a preferred embodiment the dual agonist is Hy-H[Aib]EGSFTSELATILD[K([17-carboxy-heptadecanoyl]-isoGlu)]QAARDFIAWLIQHKITD-OH (Compound 18).

Alternatively, the dual agonist is Hy-H[Aib]EGTFTSELATILD[K([17-carboxy-heptadecanoyl]-isoGlu)]QAARDFIAWLIQHKITD-OH (Compound 19).

In one aspect the compound, such as compound 18, may be formulated as follows:

| | Amount | Amount | Amount per mL |
|---|---|---|---|
| Component | per mL | per mL | (placebo) |
| Cpd 18 | 2 mg | 10 mg | N/A |
| Na2HPO4 (anhydrous) | 2.65 mg | 2.65 mg | 2.65 mg |
| Disodium phosphate, | | | |
| anhydrous/Dibasic | | | |
| sodium phosphate, | | | |
| anhydrous | | | |
| NaH2PO4 (anhydrous) | 0.16 mg | 0.16 mg | 0.16 mg |
| Sodium dihydrogen | | | |
| phosphate, anhydrous/ | | | |
| Monobasic sodium | | | |
| phosphate, anhydrous | | | |
| Mannitol (D-mannitol) | 41.90 mg | 41.90 mg | 41.90 mg |
| Hydrochloric acid² | q.s | q.s | q.s |
| Sodium Hydroxide² | q.s | q.s | q.s |
| Water for Injections | To make 1 ml | To make 1 ml | To make 1 ml |

### Medical Conditions

The dual agonists described in this specification have biological activities of both GLP-1 and GLP-2.

GLP-2 induces significant growth of the small intestinal mucosal epithelium via the stimulation of stem cell proliferation in the crypts and inhibition of apoptosis on the villi (Drucker et al. Proc Natl Acad Sci USA. 1996, 93:7911-6). GLP-2 also has growth effects on the colon. GLP-2 also inhibits gastric emptying and gastric acid secretion (Wojdemann et al. J Clin Endocrinol Metab. 1999, 84:2513-7), enhances intestinal barrier function (Benjamin et al.Gut. 2000, 47:112-9.), stimulates intestinal hexose transport via the upregulation of glucose transporters (Cheeseman, Am J Physiol. 1997, R1965-71), and increases intestinal blood flow (Guan et al. Gastroenterology. 2003, 125, 136-47).

The beneficial effects of GLP-2 in the small intestine have raised considerable interest as to the use of GLP-2 in the treatment of intestinal disease or injury (Sinclair and Drucker, Physiology 2005: 357-65). Furthermore, GLP-2 has been shown to prevent or reduce mucosal epithelial damage in a wide number of preclinical models of gut injury, including chemotherapy-induced enteritis, ischemia-reperfusion injury, dextran sulfate-induced colitis and genetic models of inflammatory bowel disease (Sinclair and Drucker Physiology 2005: 357-65). The GLP-2 analogue teduglutide (Gly2-hGLP-2) is approved for treatment of short bowel syndrome under the trade names Gattex and Revestive.

GLP-1 is a peptide hormone known for its important role in glucose homeostasis. When secreted from the gastrointestinal tract in response to nutrient ingestion, GLP-1 potentiates glucose-stimulated insulin secretion from the β-cells (Kim and Egan, 2008, Pharmacol.Rev. 470-512). Furthermore, GLP-1 or it analogues has been shown to increase somatostatin secretion and suppress glucagon secretion (Holst JJ, 2007, Physiol Rev. 1409-1439).

Besides the primary actions of GLP-1 on glucose-stimulated insulin secretion, GLP-1 is also known as a key regulator of appetite, food intake, and body weight. Moreover, GLP-1 can inhibit gastric emptying and gastrointestinal motility in both rodents and humans, most likely through GLP-1 receptors present in the gastrointestinal tract (Holst JJ, 2007, Physiol Rev. 1409-1439; Hellström et al., 2008, Neurogastroenterol Motil. Jun; 20(6):649-659). In addition, GLP-1 seems to have insulin-like effects in major extrapancreatic tissues, participating in glucose homeostasis and lipid metabolism in tissues such as muscle, liver, and adipose tissues (Kim and Egan, 2008, Pharmacol.Rev. 470-512).

The dual agonist compounds described herein find use, inter alia, in reducing or inhibiting weight gain, reducing rate of gastric emptying or intestinal transit, reducing food intake, reducing appetite, or promoting weight loss. The effect on body weight may be mediated in part or wholly via reducing food intake, appetite or intestinal transit.

Thus the dual agonists can be used for the prophylaxis or treatment of obesity, morbid obesity, obesity-linked gallbladder disease and obesity-induced sleep apnea.

As discussed above, it was surprisingly found that the particular dosage regime according to the invention was effective in reducing appetite in patients, without also resulting in the expected side effects of nausea and vomiting.

Effects on body weight may be therapeutic or cosmetic.

In a further aspect there is provided a therapeutic kit comprising a dual agonist according to the invention, or a pharmaceutically acceptable salt or solvate thereof for use in a method of reducing or inhibiting weight gain, reducing food intake, reducing appetite, promoting weight loss, or treating obesity, morbid obesity, obesity-linked gallbladder disease, or obesity-induced sleep apnea; wherein the method comprises administering the dual agonist to the patient at a dose of about 0.1 mg to about 8.0 mg.

The following examples are provided to illustrate preferred aspects of the invention and are not intended to limit the scope of the invention in any way.

### EXAMPLES

The following examples are provided to illustrate preferred aspects of the invention and are not intended to limit the scope of the invention.

### Materials and methods

The GLP-1/GLP-2 dual agonists were prepared according to the guidance in patent application publication WO2018/104561, which describes the compounds, their preparation and purification as well as analysis in detail in, for example, Examples 1 to 4.

### Example 1: GLP-1R and GLP-2R EC₅₀ Measurements

### Generation of Cell Line Expressing Human GLP-1 Receptors.

The cDNA encoding the human glucagon-like peptide 1 receptor (GLP-1R) (primary accession number P43220) was cloned from the cDNA BC112126(MGC:138331/IMAGE:8327594). The DNA encoding the GLP-1-R was amplified by PCR using primers encoding terminal restriction sites for subcloning. The 5′-end primers additionally encoded a near Kozak consensus sequence to ensure efficient translation. The fidelity of the DNA encoding the GLP-1-R was confirmed by DNA sequencing. The PCR products encoding the GLP-1-R were subcloned into a mammalian expression vector containing a neomycin (G418) resistance marker. The mammalian expression vectors encoding the GLP-1-R were transfected into HEK293 cells by a standard calcium phosphate transfection method. 48 hours post- transfection, cells were seeded for limited dilution cloning and selected with 1 mg/ml G418 in the culture medium. Following 3 weeks in G418 selection clones were picked and tested in a functional GLP-1 receptor potency assay as described below. One clone was selected for use in compound profiling.

### Generation of Cell Line Expressing Human GLP-2 Receptors

The hGLP2-R was purchased from MRC-geneservice, Babraham, Cambridge as an Image clone: 5363415 (11924-117). For subcloning into a mammalian expression vector, primers for subcloning were obtained from DNA-Technology, Risskov, Denmark. The 5′ and 3′ primers used for the PCR reaction include terminal restriction sites for cloning and the context of the 5′ primer is modified to a Kozak consensus without changing the sequence of the product encoded by the ORF. A standard PCR reaction was run using Image clone 5363415 (11924-117) as a template with the above mentioned primers and Polymerase Herculase II Fusion in a total vol. of 50 μl. The generated PCR product was purified using GFX PCR and Gel band purification kit, digested with restriction enzymes and cloned into the mammalian expression vector using Rapid DNA Ligation Kit. Ligation reaction was transformed to XL10 Gold Ultracompetent cells and colonies were picked for DNA production using Endofree Plasmid maxi kit. Subsequent sequence analysis was conducted by MWG Eurofins, Germany. The clone was confirmed to be the hGLP-2 (1-33) receptor, splice variant rs17681684.

HEK293 cells were transfected using the Lipofectamine PLUS transfection method. The day before transfection, HEK293 cells were seeded in two T75 flasks at a density of 2×10⁶ cells/T75 flask in cell culturing medium without antibiotics. On the day of transfection, cells were washed with 1× DPBS and medium was replaced with Optimem to a volume of 5 mL/T75 flask before addition of Lipofectamine-plasmid complexes were added gently and drop wise to the cells in T75 flasks and replaced with growth medium after 3 hours and again to growth medium supplemented with 500 μg/mL G418 after 24 hours. Following 4 weeks in G418 selection, clones were picked and tested in a functional GLP-2 receptor potency assay as described below. One clone was selected for use in compound profiling.

GLP-1R and GLP-2 receptor potency assays.

The cAMP AlphaScreen® assay from Perkin Elmer was used to quantitate the cAMP response to activation of the GLP1 and GLP2 receptor, respectively. Exendin-4 was used as reference compound for GLP1 receptor activation and Teduglutide as reference compound for GLP2 receptor activation. Data from test compounds eliciting an increase in the intracellular level of cAMP were normalized relative to the positive and negative control (vehicle) to calculate the EC₅₀ and maximal response from the concentration response curve. The results are listed in Table 1.

The following reference compounds A and B were also synthesised:
A Hy-H[Aib]DGSFSSELATILD[K([17-carboxy-heptadecanoyl]-isoGlu)]QAARDFIAWLIQHKITD-OH
B Hy-H[Aib]EGSFSSELATILD[K([17-carboxy-heptadecanoyl]-isoGlu)]QAARDFIAWLIQHKITD-OH

TABLE 1
EC₅₀ measurements: N/A = no detectable activity

| Compound | EC₅₀ GLP-1 (nM) | EC₅₀ GLP-2 (nM) |
|---|---|---|
| Teduglutide | 39 | 0.027 |
| Liraglutide | 0.029 | N/A |
| A | 0.490 | 0.083 |
| B | 3.900 | 0.280 |
| 1 | 0.630 | 0.350 |
| 2 | 0.130 | 0.250 |
| 3 | 0.042 | 0.330 |
| 4 | 0.660 | 0.087 |
| 5 | 0.170 | 0.063 |
| 6 | 0.058 | 0.120 |
| 7 | 0.920 | 0.019 |
| 8 | 0.220 | 0.039 |
| 9 | 0.056 | 0.056 |
| 10 | 1.800 | 0.087 |
| 11 | 0.320 | 0.085 |
| 12 | 0.140 | 0.110 |
| 13 | 2.200 | 0.099 |
| 14 | 0.570 | 0.086 |
| 15 | 0.250 | 0.160 |
| 16 | 0.073 | 0.680 |
| 17 | 0.900 | 0.330 |
| 18 | 0.190 | 0.210 |
| 19 | 0.066 | 0.230 |
| 20 | 0.550 | 0.370 |
| 21 | 1.800 | 0.270 |
| 22 | 0.230 | 0.200 |
| 23 | 0.130 | 0.240 |
| 24 | 0.210 | 0.170 |
| 25 | 0.094 | 0.330 |
| 26 | 0.290 | 0.590 |
| 27 | 0.450 | 1.100 |
| 28 | 0.360 | 0.510 |
| 29 | 0.310 | 0.290 |
| 30 | 0.310 | 0.380 |
| 31 | 0.270 | 0.240 |
| 32 | 0.380 | 0.460 |
| 33 | 0.850 | 0.072 |
| 34 | 0.280 | 0.130 |
| 35 | 0.099 | 0.300 |
| 36 | 0.320 | 3.200 |
| 38 | 0.250 | 0.890 |
| 39 | 0.044 | 0.980 |
| 40 | 0.074 | 0.500 |
| 41 | 0.048 | 0.620 |
| 42 | 0.067 | 0.330 |
| 43 | 0.096 | 0.150 |
| 44 | 0.063 | 0.140 |
| 45 | 1.400 | 0.360 |
| 46 | 0.260 | 0.380 |
| 47 | 0.440 | 0.048 |
| 48 | 0.470 | 0.054 |
| 49 | 0.270 | 0.044 |
| 50 | 0.310 | 0.056 |
| 51 | 0.020 | 0.180 |
| 52 | 0.020 | 0.075 |
| 53 | 0.076 | 0.240 |
| 54 | 0.034 | 0.990 |
| 55 | 0.110 | 0.780 |
| 56 | 0.033 | 0.076 |
| 57 | 0.093 | 0.083 |
| 58 | 0.089 | 0.090 |
| 59 | 0.088 | 0.110 |
| 60 | 0.097 | 0.074 |
| 61 | 0.130 | 0.200 |
| 62 | 0.270 | 0.150 |
| 63 | 0.310 | 0.170 |
| 64 | 0.490 | 0.200 |
| 65 | 0.130 | 0.350 |
| 66 | 0.650 | 0.180 |
| 67 | 0.160 | 0.220 |
| 68 | 0.084 | 0.100 |

### Example 2: Single Ascending Dose (SAD) Phase 1a Trials of Cpd. 18 P_{GP}-40,_{T1},M

A single ascending dose Phase 1a trial was conducted for Cpd. 18 investigating the safety of single subcutaneous injections of doses ranging from 0.02 mg to 7.5 mg in healthy human subjects.

### Trial Design

The phase 1a study was a First in Human, Randomized, Double-blind, Placebo-controlled, Single Ascending Dose Trial assessing Safety, Tolerability, Pharmacokinetics and Pharmacodynamics of a Single Subcutaneous Dose of Cpd. 18 in Healthy human Subjects.

Eight cohorts (dose levels: 0.02, 0.07, 0.2, 0.6, 1.5, 3.0, 6.0 and 7.5 mg) were given in this first in human trial. Eight subjects were allocated to the following ascending dose levels: 0.02, 0.07, 0.2, 0.6, 1.5, 3.0, 6.0 and 7.5 mg. Subjects were randomized 3:1 within each cohort, so there were 2 on placebo (PBO) and 6 on active drug in each cohort as seen in the Table 3 below.

Safety evaluation were performed after each cohort. The formulation of compound 18 and the placebo is shown in below Table 2.

TABLE 2
formulation used in the phase 1a trial.

| | Amount | Amount | Amount per mL |
|---|---|---|---|
| Component | per mL | per mL | (placebo) |
| Cpd 18 | 2 mg | 10 mg | N/A |
| Na2HPO4 (anhydrous) | 2.65 mg | 2.65 mg | 2.65 mg |
| Disodium phosphate, | | | |
| anhydrous/Dibasic | | | |
| sodium phosphate, | | | |
| anhydrous | | | |
| NaH2PO4 (anhydrous) | 0.16 mg | 0.16 mg | 0.16 mg |
| Sodium dihydrogen | | | |
| phosphate, anhydrous/ | | | |
| Monobasic sodium | | | |
| phosphate, anhydrous | | | |
| Mannitol (D-mannitol) | 41.90 mg | 41.90 mg | 41.90 mg |
| Hydrochloric acid² | q.s | q.s | q.s |
| Sodium Hydroxide² | q.s | q.s | q.s |
| Water for Injections | To make 1 ml | To make 1 ml | To make 1 ml |

TABLE 3
cohort dosing.

| | | Number of | | Number of |
|---|---|---|---|---|
| Cohort | Cpd. 18 | subjects | Placebo | subjects |
| 1 | 0.02 mg | 6 | | 2 |
| 2 | 0.07 mg | 6 | | 2 |
| 3 | 0.2 mg | 6 | | 2 |
| 4 | 0.6 mg | 6 | | 2 |
| 5 | 1.5 mg | 6 | | 2 |
| 6 | 3.0 mg | 6 | | 2 |
| 7 | 6.0 mg | 6 | | 2 |
| 8 | 7.5 mg | 6 | | 2 |

TABLE 4
Baseline characteristics.

| | Category/ | | | Category/ | |
|---|---|---|---|---|---|
| Parameter | Statistic | Overall | Parameter | Statistic | Overall |
| SEX | Male | 45 (70.3%) | Height | N | 64 |
| | Female | 19 (29.7%) | (cm) | Mean | 175.28 |
| Race | White | 61 (95.31%) | | SD | 8.820 |
| | Asian | 2 (3.13%) | | Min | 155.0 |
| | Blask Or | 1 (1.56%) | | Median | 176.00 |
| | Africa | | | Max | 202.0 |
| Age | N | 64 | BMI | N | 64 |
| (years) | Mean | 36.1 | (kg/m\hat{ }2) | Mean | 24.56 |
| | SD | 8.74 | | SD | 2.422 |
| | Min | 20 | | Min | 19.0 |
| | Median | 35.0 | | Median | 24.60 |
| | Max | 55 | | Max | 28.0 |
| Weight | N | 64 | | | |
| (kg) | Mean | 75.54 | | | |
| | SD | 10.192 | | | |
| | Min | 60.0 | | | |
| | Median | 74.50 | | | |
| | Max | 108.6 | | | |

The baseline characteristics of the subjects are given in Table 4.

Adverse events (AE) were captured by asking open ended and nonleading questions according to the following wording in the protocol:

### “9.2 Collection, Recording and Reporting of Adverse Events

All events meeting the definition of an AE must be collected and reported from the first trial-related activity after the subject has signed the informed consent and until the end of the post-treatment follow-up period. At each contact with the site (Visit or telephone) the subject must be asked about AEs. All AEs, either observed by the Investigator or reported by the subject, must be recorded by the Investigator and evaluated.
The Investigator should record the diagnosis, if possible. If no diagnosis can be made the Investigator should record each sign and symptom as individual AEs.
All AEs must be recorded by the Investigator. One single Adverse Event Form must be used per AE from start to resolution. For Serious Adverse Events (SAE), the Serious Adverse Event Form must also be completed.”

The safety data (incidence/number of subjects) for subjects in cohort 0.02 mg to 7.5 mg is shown in Table 5 below.

TABLE 5

| Dose (mg) | 0.02 | 0.07 | 0.2 | 0.6 | 1.5 | 3.0 | 6.0 | 7.5 | Placebo |
|---|---|---|---|---|---|---|---|---|---|
| Total subjects | 6 | 6 | 6 | 6 | 6 | 6 | 6 | 6 | 16 |

Gastrointestinal disorders

| | | | | | | | | | |
|---|---|---|---|---|---|---|---|---|---|
| Subjects | 2 | 0 | 1 | 0 | 0 | 3 | 4 | 2 | 2 |
| experiencing | | | | | | | | | |
| nausea | | | | | | | | | |
| Subjects | 0 | 0 | 0 | 0 | 0 | 2 | 5 | 2 | 2 |
| experiencing | | | | | | | | | |
| vomiting | | | | | | | | | |

Metabolism and nutritional disorders

| | | | | | | | | | |
|---|---|---|---|---|---|---|---|---|---|
| Subjects | 0 | 1 | 0 | 0 | 3 | 2 | 3 | 6 | 1 |
| experiencing | | | | | | | | | |
| decreased | | | | | | | | | |
| appetite | | | | | | | | | |

In this trial, half (3/6) of the subjects receiving a dose of 1.5 mg of Cpd. 18 stated that they had decreased appetite in the dose cohort and this was collected as adverse events. This continued over the next cohorts (receiving 3.0 to 6.0 mg of Cpd. 18) and in the last cohort (receiving 7.5 mg) everyone receiving Cpd. 18 reported this. The adverse events of decreased appetite can be interpreted as a marker of the satiety effects of Cpd. 18. The gastrointestinal adverse events on nausea and vomiting were only reported in the 3.0 mg cohort and for cohorts receiving higher doses than 3.0 mg.

The observation that decreased appetite without accompanying nausea or vomiting in subjects administered Cpd. 18 was entirely unexpected given the widely observed side-effects of nausea and vomiting when administering GLP-1 agonists.

The data generated with Cpd. 18 suggests that the effects on appetite reduction occurs before (at lower doses) nausea and vomiting occurs. This contrasts with the trial with semaglutide where the gastrointestinal adverse events occur before (at lower dose) the decreased satiety.

This suggests that Cpd. 18 could have a better safety profile with regards to gastrointestinal adverse events in indications where appetite reduction is desired.

### Example 3: Plasma Half-Life in Single Ascending Dose (SAD) Phase 1a Trials

Blood sampling for determining Cpd 18 plasma concentrations were performed during the phase la study (outlined in Example 2) at scheduled timepoints. Concentration of Cpd 18 in plasma was measured using a validated LC-MS/MS assay. Pharmacokinetic (PK) endpoints for Cpd 18 were derived from the individual concentration profiles (with hour as time unit). For PK analysis, Cpd 18 concentrations were supplied by the analytical laboratory in nmol/L. Mean measured concentrations per dose level are shown in Error! Reference source not found.

For determination of λ_{z} a linear regression was performed using the logarithm to plasma Cpd. 18 concentration as the response variable and at least three valid concentration measurements of the terminal end period after Cₘₐₓ. (The exact number of data points depends on the best goodness-of-fit). Cₘₐₓ is not included in the calculation for λ_{z} as this point may be affected by absorption still taking place from the injection site.

The terminal elimination half-life of the plasma Cpd. 18 profile (t½) was calculated by the following formula:

t1/2=In2/λz

As shown in Table 6, the calculated mean half-life ranges between 110 and 135 following a single dose of Cpd. 18. This would be suitable for once weekly dosing in humans.

TABLE 6
Calculated mean half-life of Cpd. 18 following single
dose in healthy subjects

| | Cpd 18 Dose Group | | | | | | | |
|---|---|---|---|---|---|---|---|---|
| t1/2 | 0.02 | 0.07 | 0.2 | 0.6 | 1.5 | 3 | 6 | 7.5 |
| (h) | mg | mg | mg | mg | mg | mg | mg | mg |
| N | 6 | 6 | 6 | 6 | 6 | 6 | 6 | 6 |
| Mean | 140 | 126 | 129 | 110 | 120 | 120 | 112 | 135 |

### Example 4: Multi Ascending Dose (MAD) Design

A single ascending Phase 1a dose trial will be conducted for Cpd. 18 investigating the safety of multiple subcutaneous injections of various ascending doses in healthy human subjects. The trial will be a First in Human, Randomized, Double-blind, Placebo-controlled, Multi Ascending Dose Trial Assessing Safety, Tolerability, Pharmacokinetics and Pharmacodynamics of Multiple Subcutaneous Dose of Cpd. 18 in Healthy human Subjects.

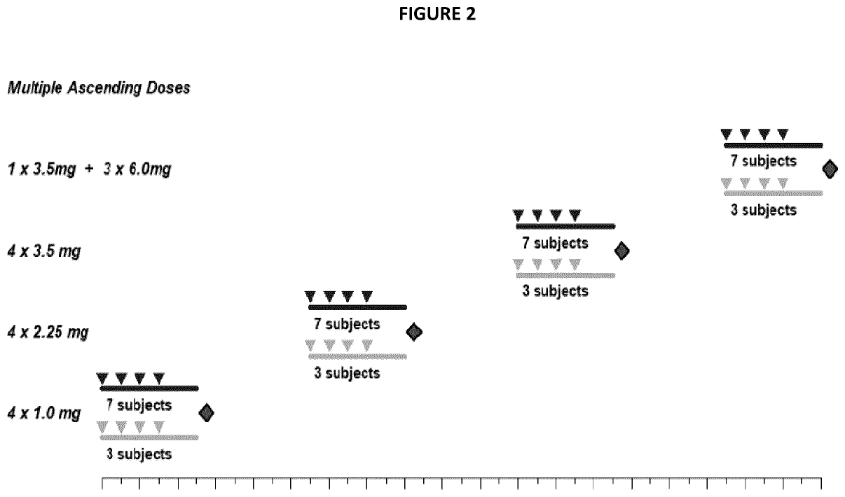

The study design is shown in FIG. 2.

Four cohorts will be given. Ten subjects will be allocated to the following ascending dose levels: 4×1.0 mg; 4×2.25 mg; 4×3.5 mg and 1×3.5 mg plus 3×6.0 mg. Subjects will be randomized within each cohort, so there will be 3 on placebo (PBO) and 7 on active drug in each cohort.

Safety evaluation will be performed after each cohort. The formulation of compound 18 and the placebo is shown in Table 2, above. Cohort dosing is shown in Table 7.

TABLE 7

| | | Number of | | Number of |
|---|---|---|---|---|
| Cohort | Cpd. 18 | subjects | Placebo | subjects |
| 1 | 4 × 1.0 mg | 7 | | 3 |
| 2 | 4 × 2.225 mg | 7 | | 3 |
| 3 | 4 × 3.5 mg | 7 | | 3 |
| 4 | 1 × 3.5 mg plus | 7 | | 3 |
| | 3 × 6.0 mg | | | |

Adverse events (AE) will be captured by asking open ended and nonleading questions according to the following wording in the protocol:

### “9.2 Collection, Recording and Reporting of Adverse Events

All events meeting the definition of an AE must be collected and reported from the first trial-related activity after the subject has signed the informed consent and until the end of the post-treatment follow-up period. At each contact with the site (Visit or telephone) the subject must be asked about AEs. All AEs, either observed by the Investigator or reported by the subject, must be recorded by the Investigator and evaluated.
The Investigator should record the diagnosis, if possible. If no diagnosis can be made the Investigator should record each sign and symptom as individual AEs.
All AEs must be recorded by the Investigator. One single Adverse Event Form must be used per AE from start to resolution. For Serious Adverse Events (SAE), the Serious Adverse Event Form must also be completed.”

### Example 5: Multi Ascending Dose (MAD) Design

A multiple ascending dose Phase 1b trial was conducted for Cpd. 18 investigating the safety of multiple subcutaneous injections of various ascending doses in healthy human subjects. The trial was a Randomized, Double-blind, Placebo-controlled, Multi Ascending Dose Trial assessing Safety, Tolerability, Pharmacokinetics and Pharmacodynamics of Multiple Subcutaneous Dose of Cpd. 18 in Healthy human Subjects.

The study design is shown in FIG. 2.

Four cohorts were dosed with four once weekly subcutaneous administrations. In each cohort, consisting of 10 subjects each, subjects were randomized to either placebo (n=3) or to the following active multiple ascending dose levels (n=7): 4 weekly dose injections of 1.0 mg; 4 weekly dose injections of 2.25 mg; 4 weekly dose injections of 3.5 mg and 1 weekly dose injections of 3.5 mg followed by 3 weekly dose injections of 6.0 mg. The formulation of compound 18 and the placebo is shown in Table 2, in Example 2 above. Cohort dosing is shown in Table 8.

TABLE 8

| | | Number of | | Number of |
|---|---|---|---|---|
| Cohort | Cpd. 18 | subjects | Placebo | subjects |
| 1 | 4 × 1.0 mg | 7 | | 3 |
| 2 | 4 × 2.25 mg | 7 | | 3 |
| 3 | 4 × 3.5 mg | 7 | | 3 |
| 4 | 1 × 3.5 mg plus | 7 | | 3 |
| | 3 × 6.0 mg | | | |

Adverse events (AE) were captured by asking open ended and non-leading questions according to the following wording in the protocol:

### 9.2 Collection, Recording and Reporting of Adverse Events

All events meeting the definition of an AE must be collected and reported from the first trial-related activity after the subject has signed the informed consent and until the end of the post-treatment follow-up period. At each contact with the site (Visit or telephone) the subject must be asked about AEs. All AEs, either observed by the Investigator or reported by the subject, must be recorded by the Investigator and evaluated.
The Investigator should record the diagnosis, if possible. If no diagnosis can be made the Investigator should record each sign and symptom as individual AEs.
All AEs must be recorded by the Investigator. One single Adverse Event Form must be used per AE from start to resolution. For Serious Adverse Events (SAE), the Serious Adverse Event Form must also be completed.”

### Example 6: Multiple Ascending Dose Phase 1b Trials of Cpd. 18

The design of this study is outlined in Example 5.

The baseline characteristics of the subjects are given in Table 9.

TABLE 9
Baseline characteristics.

| | Category/ | | | Category/ | |
|---|---|---|---|---|---|
| Parameter | Statistic | Overall | Parameter | Statistic | Overall |
| SEX | Male | 39 (97.5%) | Height | N | 40 |
| | Female | 1 (2.5%) | (cm) | Mean | 178.6 |
| Race | White | 39 (97.50%) | | SD | 6.38 |
| | Asian | 1 (2.50%) | | Min | 164 |
| Age | N | 40 | | Median | 178.0 |
| (years) | Mean | 34.1 | | Max | 193 |
| | SD | 8.80 | BMI | N | 40 |
| | Min | 19 | (kg/m\hat{ }2) | Mean | 24.56 |
| | Median | 33.0 | | SD | 2.322 |
| | Max | 53 | | Min | 19.4 |
| Weight | N | 40 | | Median | 24.80 |
| (kg) | Mean | 78.28 | | Max | 27.9 |
| | SD | 8.189 | | | |
| | Min | 65.0 | | | |
| | Median | 77.45 | | | |
| | Max | 96.5 | | | |

The safety data (incidence/number of subjects) for subjects dosed 4×1.0 mg to 1×3.5 mg plus 3×6.0 mg as well as placebo is shown in Table 10 below.

TABLE 10

| | | | | 1 × 3.5 mg plus | |
|---|---|---|---|---|---|
| Dose (mg) | 1.0 mg | 2.25 mg | 3.5 mg | 3 × 6.0 mg | Placebo |
| Total subjects | 7 | 7 | 7 | 7 | 12 |

Gastrointestinal disorders

| | | | | | |
|---|---|---|---|---|---|
| Subjects | 1 | 1 | 0 | 4 | 1 |
| experiencing | | | | | |
| nausea | | | | | |
| Subjects | 0 | 0 | 0 | 3 | 0 |
| experiencing | | | | | |
| vomiting | | | | | |

Metabolism and nutritional disorders

| | | | | | |
|---|---|---|---|---|---|
| Subjects | 0 | 3 | 2 | 6 | 0 |
| experiencing | | | | | |
| decreased | | | | | |
| appetite | | | | | |

The safety data collected in this trial supports the findings in the SAD study, which are described in Example 2. In the lower doses (2.25 mg and 3.5 mg of Cpd. 18), decreased appetite was reported by 3/7 (3 subjects out of 7 subjects) and 2/7, respectively, while only one subject reported nausea. In the highest dose, most subjects (6/7) reported decreased appetite, but nausea and vomiting were also frequently reported. The adverse events of decreased appetite following dosing of Cpd. 18 can be interpreted as a marker of the satiety effects of Cpd. 18.

The observation that decreased appetite following dosing with 2.25 mg and 3.5 mg with limited accompanying nausea or vomiting in subjects administered Cpd. 18 was entirely unexpected given the widely observed side-effects of nausea and vomiting when administering GLP-1 agonists.

The data generated with Cpd. 18 suggests that the effects on appetite reduction occurs before (at lower doses) nausea and vomiting occur. This contrasts with the trial with semaglutide (Granhall et aL., Clin Pharmacokinet 58, 781-791 (2019)), where the gastrointestinal adverse events occur before (at lower dose) the decreased appetite.

This suggests that Cpd. 18 could have a better safety profile with regards to gastrointestinal adverse events in indications where appetite reduction is desired.

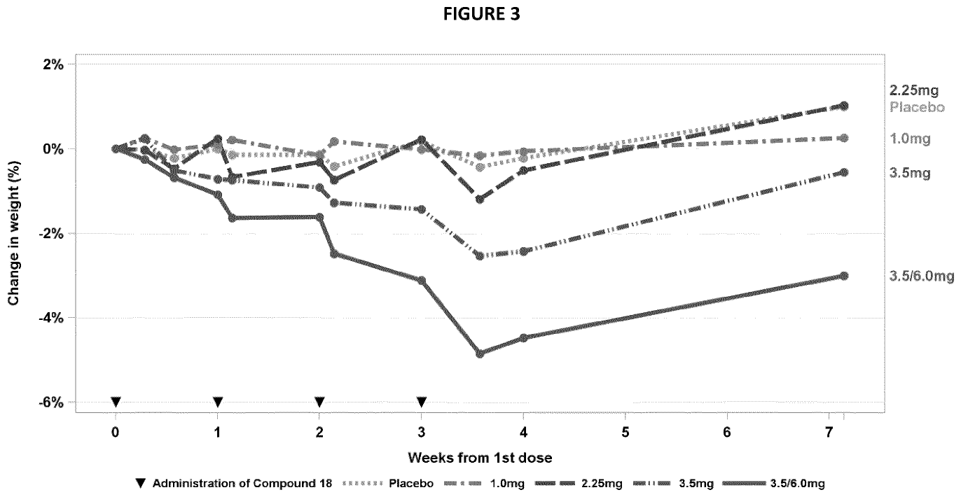

Body weight was measured throughout the study and showed a dose dependent reduction in body weight, see FIG. 3.

Reduction in appetite is reflected in the reduction in food intake measured by mixed meal test for breakfast (Table 11) and by measuring caloric intake at standard meals for lunch and dinner (Table 12).

The Mixed Meal Test (MMT) was performed at baseline, at 24 hours, after the first dosing (Day 2) and after the fourth dosing (Day 23). The meal consisted of a fixed nutrient content and the exact initial amount of nutrients were weighed by kitchen staff on a lab scale using the method of weighed intake. Consumption was supervised and leftovers were weighed and recorded as percentage of meal. Adjustment of leftovers on Day 2 will be performed for Day 23 and differences in weight will be calculated in carbohydrates.

Pre-defined lunch and dinner meals were served at baseline (on Day -1) and after 4^{th} dose (Day 23). Consumption was supervised and leftovers were weighed.

Table 11 shows food consumption data from the Mixed Meal Test.

TABLE 11
Mixed meal test, breakfast.

| | | | Number of | Mean food |
|---|---|---|---|---|
| Mixed meal test | | | subjects | consumption |
| (MMT) | Cohort | | observed | (%) |
| Baseline | Placebo | | 12 | 100 |
| | 1.0 | mg | 7 | 99.2 |
| | 2.25 | mg | 7 | 100 |
| | 3.5 | mg | 7 | 100 |
| | 3.5/6.0 | mg | 7 | 95.8 |
| After 1^{st} dose | Placebo | | 12 | 99.6 |
| | 1.0 | mg | 7 | 99.0 |
| | 2.25 | mg | 7 | 95.4 |
| | 3.5 | mg | 7 | 96.2 |
| | 3.5/6.0 | mg | 7 | 87.5 |
| After 4^{th} dose | Placebo | | 12 | 99.1 |
| | 1.0 | mg | 7 | 98.6 |
| | 2.25 | mg | 7 | 95.8 |
| | 3.5 | mg | 7 | 93.2 |
| | 3.5/6.0 | mg | 7 | 62.5 |

It can be seen that at baseline the subjects in all dose levels incl. placebo, consumed the majority of the meal since the mean percentage consumption is 95.8-100% However, after the 1^{st} dose there was a dose dependent reduction in food consumption as the two highest cohorts showed food consumption of 99.2 and 87.5%, respectively. Further, after the 4^{th} dose there was a large reduction in food consumption in the highest dose level, showing 62.5% consumption.

Table 12 shows food consumption of the fixed meals served at lunch and dinner at baseline and after 4^{th} dose. The data shows a consistent dose-dependent reduction in food consumption of a similar magnitude as seen for the Mixed Meal Test.

TABLE 12
Standard meal test, lunch, and dinner

| | | |
|---|---|---|
| | Number of | Mean food |
| | subjects | consumption |
| Cohort | observed | (kcal) |

Lunch

| Baseline | Placebo | | 12 | 688 |
|---|---|---|---|---|
| | 1.0 | mg | 7 | 701 |
| | 2.25 | mg | 7 | 638 |
| | 3.5 | mg | 7 | 706 |
| | 3.5/6.0 | mg | 7 | 671 |
| After 4^{th} dose | Placebo | | 12 | 700 |
| | 1.0 | mg | 6 | 705 |
| | 2.25 | mg | 7 | 649 |
| | 3.5 | mg | 7 | 647 |
| | 3.5/6.0 | mg | 7 | 403 |

Dinner

| Baseline | Placebo | | 12 | 694 |
|---|---|---|---|---|
| | 1.0 | mg | 7 | 694 |
| | 2.25 | mg | 7 | 679 |
| | 3.5 | mg | 7 | 695 |
| | 3.5/6.0 | mg | 7 | 694 |
| After 4^{th} dose | Placebo | | 11 | 696 |
| | 1.0 | mg | 6 | 682 |
| | 2.25 | mg | 7 | 585 |
| | 3.5 | mg | 7 | 594 |
| | 3.5/6.0 | mg | 7 | 423 |

### Example 7: Clinical Trial Investigating Weight Loss for Compound 18

This study will investigate the efficacy of once-weekly subcutaneously administered Cpd 18 in obese individuals.

The primary aim is to compare the effect of 4 mg and 6 mg Cpd 18 versus placebo on change in body weight (%) from baseline during a 12-week treatment period.

Secondary and exploratory aims include evaluating the effects of 4 mg and 6 mg Cpd 18 versus placebo after 12 weeks of treatment on gut barrier function, safety and tolerability and patient-reported outcomes.

### Study Design

This study is a proof-of-concept, randomised, double-blind, placebo-controlled, parallel-group, single-centre clinical trial investigating the body weight loss potential of Cpd 18, administered once weekly.

Eligible subjects will be randomised to one of three treatment arms:

TABLE 13
Overview of treatment arms, dose, usage, route- and frequency
of product administration.

| | | | | | Adminis- |
|---|---|---|---|---|---|
| Treatment | | | Pharmaceutical | Route of | tration |
| arm | IMPs | Dose | dosage form | administration | frequency |
| #1 | Cpd 18 | 4 mg | 1 mL | Sub- | Once |
| | (10 mg/ml) | | solution for | cutaneously | weekly |
| #2 | Cpd 18 | 6 mg | injection | | Once |
| | (10 mg/ml) | | in vial | | weekly |
| #3 | Placebo | 4 mg | | | Once |
| | (n/a) | 6 mg | | | weekly |
| | | | | | Once |
| | | | | | weekly |

In total, 54 obese participants (18-75 years) with a body mass index (BMI) of 30 kg/m² will be randomised to either treatment with the investigational medicinal product (IMP), being either compound 18 at 4 mg, compound 18 at 6 mg, or placebo, for 12 weeks. To ensure blinding, the placebo arm is split between 4 mg and 6 mg placebo, making the randomisation sequence 2:2:1:1. The trial encompasses a 3-week screening period containing a screening visit (V1) to assess eligibility, followed by a randomisation visit (V2) and subsequently a 12-week treatment period concluded with a 4-week follow-up period. The IMP will be subcutaneously administered in the abdomen once weekly from week 0 (V2) until week 12 (V14) (Table 14).

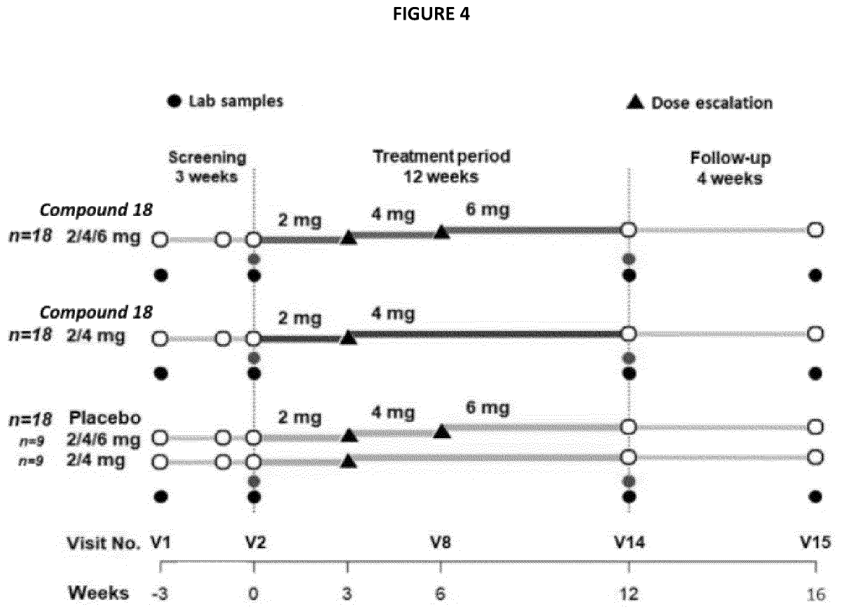

The IMP will be initiated at 2 mg once-weekly and up-titrated every third week by 2 mg until the respective trial doses are reached in each arm (FIG. 4). Hereafter, the participants will be kept at the dose level for the remainder of the trial (from week 3 and week 6 for the 4 mg and 6 mg doses, respectively). However, to reduce dropout in cases of low tolerability of the IMP, the investigator can postpone up-titration or down-titrate if judged necessary for participant retention or safety. The trial schedule will consist of five on-site visits, including screening, randomisation and a safety follow-up visit (four weeks after end of treatment (EOT)), in addition to a minimum of 10 telephone consultations. Therefore, the maximum trial duration will be 16 weeks. A maximum of n=7 from each treatment arm (total n=21) can participate in this sub-study.

TABLE 14
Outline of trial

| | | | | | | | | | | | | | | End | End |
|---|---|---|---|---|---|---|---|---|---|---|---|---|---|---|---|
| | | | | | | | | | | | | | | of | of |
| | Screening | Randomisation | Dose escalation and treatment period | | | | | | | | | | | treatment | trial |
| Visit | V1 | V2 | P3 | P4 | P5 | P6 | P7 | V8 | P9 | P10 | P11 | P12 | P13 | V14 | V15 |
| Time | −3 | 0 | 1 | 2 | 3 | 4 | 5 | 6 | 7 | 8 | 9 | 10 | 11 | 12 | 16 |
| (weeks) | | | | | | | | | | | | | | | |
| Visit | −21 | ±2 | ±2 | ±2 | ±2 | ±2 | ±2 | ±2 | ±2 | ±2 | ±2 | ±2 | ±2 | ±2 | ±5 |
| window | | | | | | | | | | | | | | | |
| (days) | | | | | | | | | | | | | | | |

The endpoints of the clinical study are shown below in Table 15.

TABLE 15
Clinical endpoints.

| Endpoint title | Time frame | Unit |
|---|---|---|
| Change in body weight | From week 0 (baseline) | %-point |
| | to end of treatment (EOT) | |
| Number of participants | At EOT | Counts of |
| achieving body weight | | participants |
| reduction ≥5% (yes/no) | | |
| Change in BMI | From week 0 (baseline) to EOT | kg/m² |

The investigator is responsible for detection, documentation, recording, and follow-up of all adverse events (AE). All AEs occurring after signed informed consent (V1) until completion of the study period will be registered (V15) as illustrated in Table 14. The participants is instructed to record AEs in a diary in between site visits and study staff will enquire about AE's in an open-ended and non-leading way during weekly phone visits. All AEs will be evaluated for severity and relationship to IMP by the investigator. All types of AEs will be recorded in the case report form (CRF).

1. A method for reducing or inhibiting weight gain, reducing food intake, reducing appetite, promoting weight loss, or treating obesity, morbid obesity, obesity-linked gallbladder disease, or obesity-induced sleep apnea comprising administering to a patient a GLP-1/GLP-2 dual agonist which is:
Hy-H[Aib]EGSFTSELATILD[K([17-carboxy-
heptadecanoyl]-isoGlu)]QAARDFIAWLIQHKITD-OH
(Compound 18)
or a pharmaceutically acceptable salt thereof;
at a dose of about 1.5 mg to about 10.0 mg.

2.-4. (canceled)

5. The method according to claim 1, wherein the method comprises administering the dual agonist, or a pharmaceutically acceptable salt thereof, to the patient at a dose of about 1.5 mg to about 8.0 mg.

6. The method according to claim 1 wherein the method comprises administering the dual agonist, or pharmaceutically acceptable salt thereof, to the patient at a dose of about 1.5 to about 7.5 mg, about 1.5 to about 6.0 mg, about 1.5 to about 4.0 mg, or about 1.5 to about 3.5 mg.

7. The method according to claim 1, wherein the method comprises administering the dual agonist, or pharmaceutically acceptable salt thereof, to the patient at a dose of about 2.0 to about 7.5 mg, about 2.0 to about 6.0 mg, about 2.0 to about 4.0 mg, preferably about 2.0 to about 3.5 mg, or about 2.25 to about 3.5 mg.

8. The method according to claim 1, wherein the method comprises administering to the patient 1, 2, 3 or 4 lower doses of the dual agonist or pharmaceutically acceptable salt thereof, followed by at least one higher dose of the dual agonist or pharmaceutically acceptable salt thereof.

9. The method according to claim 8, wherein said lower dose is between about 1.5 mg and about 3.5 mg.

10. The method according to claim 8, wherein said higher dose is between about 6.0 mg and about 8.5 mg.

11. The method according to claim 1, wherein the method comprises administering the dual agonist, or pharmaceutically acceptable salt thereof, to the patient by injection, or by subcutaneous injection.

12. The method according to claim 1, wherein the patient is a human.

13. The method according to claim 1, wherein the patient does not experience side-effects of nausea and/or vomiting following administration of the dual agonist.

14. The method according to claim 1, wherein the method comprises once-weekly administration of the dual agonist.

15. The method according to claim 1, wherein said dual agonist or pharmaceutically acceptable salt thereof is in the form of a composition comprising the dual agonist in admixture with a carrier.

16. The method according to claim 1, wherein the method comprises administering the dual agonist, or pharmaceutically acceptable salt thereof, to the patient at a dose of about 1.5 mg, about 2.0 mg, about 2.25 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 9.0 mg or about 10.0 mg.

17. The method according to claim 15, wherein the composition is a pharmaceutical composition and the carrier is a pharmaceutically acceptable carrier.

18. A method according to claim 17, wherein the pharmaceutical composition is an isotonic parenteral composition.

19. The method according to claim 18, wherein the pharmaceutical composition is an isotonic parenteral composition comprising:
a) about 5 mM to about 50 mM of phosphate buffer component, or about 10 mM to about 40 mM, or about 15 mM to about 30 mM, or about 20 mM of phosphate buffer component; and
b) about 190 mM to about 240 mM of one or more tonicity agent, wherein said one or more tonicity agent comprises or is a non-ionic tonicity agent, and wherein the non-ionic tonicity agent is mannitol,
wherein said composition further comprises a solvent, and
wherein said composition has a pH of about pH 6.0 to about pH 8.2, or a pH of about pH 7.0 to about pH 8.0.
